(19) United States
(12) Patent Application Publication
(10) Pub. No.: US 2023/0118467 A1
(43) Pub. Date: Apr. 20, 2023

(54) COSMETIC OR PHARMACEUTICAL USE OF AVENANTHRAMIDE L

(71) Applicant: SYMRISE AG, Holzminden (DE)

(72) Inventors: Martina Herrmann, Hameln (DE); Gerhard Schmaus, Höxter-Bosseborn (DE); Nikolas Bugdahn, Holzminden (DE); Dominik Stuhlmann, Holzminden (DE); Katharina Striewe, Kassel (DE); Holger Joppe, Dassel (DE)

(21) Appl. No.: 17/909,693

(22) PCT Filed: Mar. 6, 2020
(86) PCT No.: PCT/EP2020/056119

§ 371 Date: Sep. 6, 2022

(51) Int. Cl. A61K 8/42; A61K 8/9794; A61Q 19/00; A61Q 19/02; A61Q 3/00; A61Q 5/00; A61K 31/164; A61K 36/899; A23L 33/105

(52) U.S. Cl. CPC A61K 8/42; A61K 8/9794; A61Q 19/007; A61Q 19/02; A61Q 3/00; A61Q 5/00; A61K 31/164; A61K 36/899; A23L 33/105

(57) ABSTRACT

The present invention relates generally to: the cosmetic or pharmaceutical use of avenanthramide L or an oat extract comprising avenanthramide L; avenanthramide L or an oat extract comprising avenanthramide L as a neurokinin-1 receptor NK1R antagonist; and a method for preparing of preparing avenalumic acid and/or avenanthramide L.

### TECHNICAL FIELD

The present invention relates generally to: the cosmetic or pharmaceutical use of avenanthramide L or an oat extract comprising avenanthramide L; avenanthramide L or an oat extract comprising avenanthramide L as a neurokinin-1 receptor (NK1R) antagonist; and a method for preparing avenalumic acid and/or avenanthramide L.

### BACKGROUND ART

Oatmeal has been used for centuries as a soothing agent to relieve itching and irritation associated with various xerotic dermatoses. Medical texts promoted the topical application of oatmeal flour for a variety of dermatological conditions. The most common clinical applications for colloidal oatmeal in dermatological practice are as an adjunctive therapy for pruritic skin conditions such as atopic dermatitis and allergic or irritant contact dermatitis. The direct anti-irritant activity of oats has been well established both in vitro and in clinical studies. Extracts of oats have been shown to decrease the ionophore-stimulated liberation of arachidonic acid from phospholipids in keratinocytes and inhibit prostaglandin biosynthesis. Despite the wide-spread use of skin anti-irritants, few studies have examined the phytochemicals present in oats that mediate the anti-inflammatory activity.

Oats exist in two main species, Avena sativa L. and Avena nuda L. (synonyms include Avena sativa subsp. nuda (L.) after Gillet & Magne, and Avena sativa var. nuda (L.) after Körn). A. sativa, also known as common or husked oat, is primarily grown in cool temperate climates, in particular in the cool and moist regions of Northern Europe and North America. A. nuda is known as naked or huskless oat because the husk is removed when the crop is harvested, and it has a free threshing character similar to wheat. Husked oats represent the majority of global oat production, except in China, where naked oat is the most common type.

The composition of oats is predominantly starch (65 to 85%), proteins (15 to 20%, including enzymes), lipids (3 to 11%) and about 2 to 8.5% dietary fibres including a high content of ß-glucans. Oats also contain other important bioactive compounds such as phenolic compounds.

Phenolic compounds have antioxidant properties and can protect against degenerative diseases (such as heart disease and cancer) in which reactive oxygen species (i.e. superoxide anions, hydroxyl radicals and peroxy radicals) are involved.

A general definition of a phenolic compound is any compound containing a benzene ring with one or more hydroxyl groups. Phenolic acids, flavonoids, condensed tannins, coumarins and alkylresorcinols are examples. In cereal grains, these compounds are located mainly in the pericarp, and they can be concentrated by decorticating the grain to produce bran. Phenolic compounds can be grouped into flavonoids (sub-classified as flavonols, flavones, isoflavones, anthocyanins, flavanols, flavanones, etc.) and non-flavonoids. Phenolic compounds can exist as free phenols or in glycosidic form. They tend to be relatively polar and typically dissolve in pure or aqueous alcohols such as ethanol and methanol or aqueous acetone. Many phenolic compounds in cereals (such as phenolic acids and flavonoids) are also reported in fruits and vegetables, but some phenols are unique to one plant species, such as for example oat avenanthramides.

Phenolic compounds have been shown to possess numerous activities, the most important being the antioxidant activity which prevents lipid peroxidation and cellular oxidative damage mediated by harmful free radicals. This property is related to the ability of phenolic compounds to scavenge free radicals, donate hydrogen atoms or electrons, or chelate metal cations [Dykes et al., Cereal Foods World, 2007, 105-111].

The type and concentration of phenolic compounds in wholemeal cereals are influenced by the plant variety and nature of the grain. Besides containing high levels of phenolic acids, tocopherols and alk(en)ylresorcinol derivatives, oats are in particular a unique source of avenanthramides (Avns; also known as N-cinnamoyl anthranilate alkaloids or anthranilic acid amides), which are not present in other cereals.

Avenanthramides (in the following abbreviated as Avns or Avn for a single avenanthramide compound), which are low-molecular-weight phenolic amides containing anthranilic acid and hydroxycinnamic acid moieties with an amide bond, are a group of naturally occurring phenolic amides in oats, both A. sativa and A. nuda. They were originally identified as phytoalexins produced by the plant in response to exposure to pathogens, such as fungi.

Oats contain a unique group of approximately 40 different types of Avns, which are present in both oat grains and leaves. The most abundant are Avn A (N-(4′-hydroxycinnamoyl)-5-hydroxyanthranilic acid), Avn B (N-(4′-hydroxy-3′-methoxycinnamoyl)-5-hydroxyanthranilic acid) and Avn C (N-(3′-4′-dihydroxycinnamoyl)-5-hydroxyanthranilic acid), which are amides of 5-hydroxyanthranilic acid with p-coumaric, ferulic and caffeic hydroxycinnamic acids, respectively. These Avns are constitutively expressed in the kernels, appearing in almost all milling fractions, but occur at their highest concentrations in the bran and outer layers of the kernel [Boz H., Czech Journal of Food Sciences 2015, 33(5): 399-404]. The total content of avenanthramides (Avns) in oat grain has been found to be about 2 to 700 mg/kg (0.0002 to 0.07%), depending on the cultivar and agronomic treatment [Maliarova M. et al., Journal of the Brazilian Chemical Society 2015, 26(11), 2369-2378].

A number of studies have demonstrated that Avns have strong antioxidant activity both in vitro and in vivo, as well as anti-inflammatory, anti-irritant, anti-atherogenic and anti-proliferative activities which may prevent or limit cellular oxidative dysfunctions and the development of oxidative stress-related diseases, such as neurodegenerative and cardiovascular diseases, and provide additional protection against skin irritation, aging, CHD and cancer [Perrelli A. et al., Oxidative Medicine and Cellular Longevity 2018, DOI: 10.1155/2018/6015351].

The extraction of Avns from oats was carried out using various solvent compositions such as pure or diluted ethanol and methanol. Extraction procedures were achieved over different times at room temperature or under controlled heating, such as naked oats, 50% aqueous ethanol [Tong L et al., Journal of Integrative Agriculture 2014, 13, 1809].

Maliarova, M. et al., Journal of the Brazilian Chemical Society 2015, 26(11), 2369-2378 compared the efficiency of methanol, ethanol and isopropanol on the extraction of Avns from naked oat bran. The optimum conditions for the highest yield of Avns were a methanol concentration of 70%, an extraction temperature of 55° C. and an extraction time of 165 minutes.

The antioxidant activity of Avns has been found to be 10 to 30 times higher than those of the typical cereal components ferulic acid, gentisic acid, p-hydroxybenzoic acid, protocagtechuic acid, syringic acid, vanillic acid and vanillin. The Avns differ in the antioxidant activity, Avn C having the highest activity, followed by Avn B and Avn A. Avns enriched extract of oats inhibits LDL oxidation in vitro. Both, animal studies and human clinical trials confirmed that oats antioxidants have the potential of reducing cardiovascular risks by lowering serum cholesterol, inhibiting LDL cholesterol oxidation and peroxidation. Another study has indicated that the consumption of oats and oats bran may reduce the risk of colon cancer not only because of their high fiber contents but also due to Avns. Furthermore, Avns enriched oat extracts have been shown to inhibit atherosclerosis and activation of the NF-kB transcription factor, which is the regulator of infection and inflammation [Hüiseyin Boz, Phenolic Amides (Avenanthramides) in Oats—A Review, Czech J. Food Sci., 33, 2015 (5), 399-404].

Despite widespread use in treating skin irritation, the phytochemicals present in oats and responsible for anti-inflammatory, anti-itching, anti-irritant, anti-atherogenic and anti-proliferative activities activity have not so far been elucidated.

WO 2004/047833 describes the inhibition of substance P-induced liberation of histamine from mast cells and the treatment and prevention of itching by substances of Formula 2:

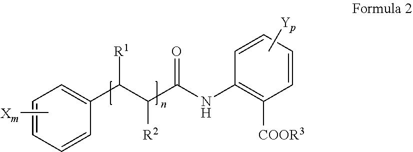

where m=0, 1, 2 or 3, p=0, 1 or 2, and n=0, 1 or 2,
with the proviso that if n=1 or 2, then p+m>0,
and if n=1 or 2, then R¹ and R², in respective pairs, respectively denote H or together denote another chemical bond (as for example in cinnamic acid derivatives),
and if m=1, 2 or 3, then each X independently denotes OH, Oalkyl or Oacyl,
and if p=1 or 2, then each Y independently denotes OH, Oalkyl or Oacyl,
and if p+m>0, then at least one of X and Y is selected from the group consisting of OH and Oacyl,
and where R³ is —H or an alkyl (in particular —CH₃, or other straight-chain or branched alkyl chains with 2 to 30 C atoms; in this context, R³ is also —H for the corresponding pharmaceutically acceptable salts).

WO 2017/159964 describes avenanthramides, including avenanthramide L, for preventing or treating hearing loss.

EP 1 574 20 describes avenanthramides, including compounds structurally related to avenanthramide L, as 5-lipoxygenase inhibitors.

Lotts T. et al., Experimental Dermatology 2017, 26(8): 739-742, describes how dihydroavenanthramide D (CAS 697235-49-7, INCI name: hydroxyphenyl propamidobenzoic acid; the active ingredient in SymCalmin® provided by Symrise) inhibits mast cell degranulation and exhibits anti-inflammatory effects through the interaction with the neurokinin-1 receptor. The activity of avenanthramides, in particular avenanthramide L, is not however described.

Ständer, S. et al., Targeting the Neurokinin Receptor 1 with Aprepitant: A Novel Antipruritic Strategy, PLOS ONE (Public Library of Science) 2010, 5(6): 0010968, describes how targeting the neuropeptide SP by applying the NKR1 antagonist aprepitant is an effective approach for the treatment of chronic pruritus.

Chronic pruritus is a frequent and globally occurring symptom of systemic, dermatologic, neurological and psychiatric diseases; its pathophysiology is still not fully understood. It is currently estimated that 20 to 27% of all adults worldwide endure chronic pruritus. Since the symptom is regularly characterised by a high intensity and long duration and by cutaneous self-injury due to scratching, it has a high impact on the quality of life of sufferers. Given that pruritus was regarded for a long time as a sub-quality of pain, not much attention has been paid in the past to the neurobiological basis of the symptom. A second reason for the lack of pursuit of specific treatment strategies was the assumption that treatment of the underlying disease would automatically relieve the symptom of pruritus. The mainstays of the treatment of chronic pruritus to date are therefore still antihistamines, topical and systemic corticosteroids or certain antidepressants. However, their efficacy is limited, and systemic application of corticosteroids and antidepressants may be associated with severe side-effects.

Pruritus is also an important feature of many dermatoses with impaired skin barrier function such as atopic dermatitis (AD) and psoriasis. The skin barrier prevents the entry of harmful agents, such as antigens and infectious microorganisms, and prevents moisture loss. Impaired barrier function has been linked to dry, itchy skin characterised by redness, flakes, cracks and a rough texture (“outside-in”), but epidermal inflammation can also weaken the barrier (“inside-out”). The underlying dermatoses associated with dry skin (xerosis) and itch can differ between patient populations. Structural and physiological changes in the skin barrier occur with age and can lead to an increased incidence of barrier abnormalities among the elderly. Xerosis is the most common cause of skin barrier related pruritus in this population and has been reported in 69% of elderly chronic itch patients. However, in children and adults, one of the most common causes of pruritus is AD, a chronic inflammatory disorder in which patients experience itch with high intensity (G. Yosipovitch et al., Acta Derm. Venereol. 2019, doi: 10.2340/00015555-3296).

Furthermore, the use of toiletries such as soaps and shampoos containing surfactants may cause adverse effects such as cutaneous irritation, dryness, and itching. Skin pathologies, including dry skin, rough skin, and sensitive skin, have increased because of changes in living conditions and lifestyle. Many people with skin pathologies complain of itching during and/or after skin washing using detergents and this was shown to be linked to histamine released from epidermal keratinocytes (Y. Inami et al., Yakugaku Zasshi 2012, 132, 1225-30).

Itch leads to scratching which worsens cutaneous barrier disruption.

There is thus an ongoing need in the cosmetics and pharmaceutical industry for the development of new agents or preparations for use in skin care or skin protection and in the prevention and/or treatment of dermatoses, in particular itch and/or itch-related dermatoses.

It should generally be borne in mind that the substances to be used in the end formulation must be
toxicologically acceptable,
well tolerated by the skin,
stable (in particular in the customary formulations),
preferably odourless and
able to be produced inexpensively (i.e. using standard processes and/or starting from standard precursors)
   in the concentration range relevant to activity and administration.

It is therefore the object of the present invention to provide for the use of such active substances or preparations for skin protection and in the prevention and/or treatment of dermatoses, in particular itch and/or itch-related dermatoses.

Surprisingly, it turns out that avenanthramide L or an oat extract comprising avenanthramide L exhibits highly interesting biological benefits, such as antioxidant, anti-inflammatory, anti-itching, anti-irritant and anti-atherogenic activities, and are thus beneficial agents for skin care and skin protection and in the prevention and/or treatment of dermatoses. In particular, it turns out that avenanthramide L or oat extract comprising avenanthramide L is an effective agent in the prevention and/or treatment of dermatoses, in particular dermatological or keratological disorders having a barrier related, inflammatory, immunoallergic, atherogenic, xerotic or hyperproliferative component. In particular, it turns out that avenanthramide L or a preparation comprising avenanthramide L is an effective agent in the prevention and/or treatment of itch and/or itch-related dermatoses.

### SUMMARY OF THE INVENTION

The primary aim of the present invention is therefore to provide for the use of avenanthramide L or an oat extract comprising avenanthramide L as an antagonist of the neurokinin-1 receptor NK1R.

In a second aspect, the present invention relates to the use of avenanthramide L or an oat extract comprising avenanthramide L for inducing the expression of small heat shock proteins or for inducing the expression of CD44.

In a third aspect, the present invention relates to the use of avenanthramide L or an oat extract comprising avenanthramide L as an antioxidant and/or for inducing the expression of BLVRB.

In a fourth aspect, the present invention relates to the use of avenanthramide L or an oat extract comprising avenanthramide L as a cosmetic for skin care, scalp care, hair care or nail care and/or for use in the prevention and/or treatment of skin conditions, intolerant and sensitive skin, skin irritation, skin reddening, wheals, pruritis (itching), skin aging, wrinkle formation, loss of skin volume, loss of skin elasticity, pigment spots, pigment abnormalities, dry skin, i.e. for moisturising the skin.

In a fifth aspect, the present invention relates to avenanthramide L or an oat extract comprising avenanthramide L for use as a medicament, in particular for use in the prevention and/or treatment of dermatological or keratological diseases, in particular dermatoses having a barrier related, inflammatory, immunoallergic, atherogenic, xerotic or hyperproliferative component, in particular itch and/or itch-related dermatoses.

In a sixth aspect, the present invention relates to the use of avenanthramide L or an oat extract comprising avenanthramide L for preparing foods, food supplements, cosmetic, pharmaceutical or veterinary preparations.

In a seventh aspect, the present invention relates to avenanthramide L or an oat extract comprising avenanthramide L as a neurokinin-1 receptor NK1R antagonist.

Finally, the present invention relates to a method for preparing avenalumic acid or avenanthramide L.

The invention is specified in the appended claims. The invention itself, and its preferred variants, other objects and advantages, are however also apparent from the following detailed description in conjunction with the accompanying examples.

### DESCRIPTION OF THE FIGURES

FIG. 1 is the ¹H NMR spectrum of methyl (2E)-4-(diethylphosphoryl-)but-2-enoate, CDCl₃, 300 MHz; E isomer (coupling constant=15 Hz)

FIG. 2 is the ¹H NMR spectrum of avenalumic acid methyl ester, CDCl₃, 300 MHz; compound 5

FIG. 3 is the ¹H NMR spectrum of avenalumic acid, DMSO-d₆, 400 MHz

FIG. 4 is the ¹³C NMR spectrum of avenalumic acid, DMSO-d₆, 101 MHz ¹³C

FIG. 5 is the LCMS spectrum of avenalumic acid

FIG. 6 is the ¹H NMR spectrum of avenanthramide L, DMSO-d₆, 400 MHz

FIG. 7 is the ¹³C NMR spectrum of avenanthramide L, DMSO-d₆, 101 MHz

FIG. 8 is the LCMS spectrum of avenanthramide L.

### DETAILED DESCRIPTION OF THE INVENTION

Within the context of the present invention, the general term “avenanthramide(s) (anthranilic acid amides)” is understood to mean a member of a group of phenolic alkaloids found mainly in oats (Avena sativa) but also present in white cabbage butterfly eggs (Pieris brassicae and P. rapae) and in fungus-infected carnations (Dianthus caryophyllus).

Avenanthramides are naturally found in and can be isolated and purified from oats. The two main species of oats are Avena sativa L. and Avena nuda L. (synonyms include Avena sativa subsp. nuda (L.) after Gillet & Magne, and Avena sativa var. nuda (L.) after Körn), wherein they appear to be most concentrated in the peripheral regions, husks, trichomes or straw. More than 50 distinct avenanthramides have been isolated from oat grains [Collins, Journal of Agricultural and Food Chemistry, 37 (1989), 60-66].

Avns can be represented by the following general Formula 1:

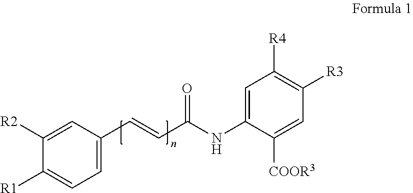

The following Table 1 shows examples of naturally occurring isolated and/or synthesised Avns based on general Formula 1.

TABLE 1

| Avenanthramide *) | CAS number | n | R1 | R2 | R3 | R4 |
|---|---|---|---|---|---|---|
| A | 108605-70-5 | 1 | OH | H | OH | H |
| B | 108605-69-2 | 1 | OH | OMe | OH | H |
| C | 116764-15-9 | 1 | OH | OH | OH | H |
| D | 115610-36-1 | 1 | OH | H | H | H |
| E | 93755-77-2 | 1 | OH | OMe | H | H |
| F | 116764-16-0 | 1 | OH | OH | H | H |
| G | 116764-17-1 | 1 | OH | H | H | OH |
| H | 116764-18-2 | 1 | OH | OMe | H | OH |
| K | 116764-19-3 | 1 | OH | OH | H | OH |
| X | 1158480-77-3 | 1 | OH | H | OH | OMe |
| Y (2 **) | 154992-25-3 | 1 | OH | OMe | OH | OMe |
| Z | 1158480-80-8 | 1 | OH | OH | OH | OMe |
| AA | 157799-28-5 | 1 | OH | H | OH | OH |
| BB | 2304718-64-5 | 1 | OH | OMe | OH | OH |
| CC | 1819995-77-1 | 1 | OH | OH | OH | OH |
| O (L **) | 172549-38-1 | 2 | OH | H | OH | H |
| P | 1358438-37-5 | 2 | OH | OMe | OH | H |
| Q | 2227208-43-5 | 2 | OH | OH | OH | H |
| L | 2301866-39-5 | 2 | OH | H | H | H |
| M | 101618-11-5 | 2 | OH | OMe | H | H |
| N | 101618-21-7 | 2 | OH | OH | H | H |
| R | 1191042-39-3 | 2 | OH | H | H | OH |
| S | 2301866-43-1 | 2 | OH | OMe | H | OH |
| T | 2301864-63-9 | 2 | OH | OH | H | OH |
| U | 2301864-86-6 | 2 | OH | H | OH | OMe |
| V | 2304718-63-4 | 2 | OH | OMe | OH | OMe |
| W | 2304718-62-3 | 2 | OH | OH | OH | OMe |
| OO | 2301866-28-2 | 2 | OH | H | OH | OH |
| PP | 2301864-57-1 | 2 | OH | OMe | OH | OH |
| QQ | 2301864-89-9 | 2 | OH | OH | OH | OH |

*) Abbreviations Collins [de Bruijn et al., Food Chemistry (2018), doi: https://doi.org/10.1016/j.foodchem.2018.11.013, supplementary information Table S1]
**) More commonly used, non-Collins abbreviations

The most abundant avenanthramides in oats are: avenanthramide A (also called 2p, AF-1 or Bp), avenanthramide B (also called 2f, AF-2 or Bf), avenanthramide C (also called 2c, AF-6 or Bc), avenanthramide L (non-Collins abbreviation; CAS number 172549-38-1) (also called avenanthramide O (Collins abbreviation) or 2pd), avenanthramide P (also called 2fd) and avenanthramide Q (also called 2 cd).

A number of studies have demonstrated that these latter avenanthramides have anti-inflammatory, antioxidant, anti-itching, anti-irritant and anti-atherogenic activity, however their underlying mechanisms and targeted molecules remain unexplained.

The naturally occurring avenanthramide compounds can alternatively also be produced by organic synthesis.

Said synthetic prepared avenanthramide substances are identical to the corresponding naturally occurring avenanthramide compounds as extracted from oats.

Non-naturally occurring avenanthramides analogues which are in accordance with the following general Formula 2 and endowed with important biological properties have been artificially produced by organic synthesis methodologies, such as for example those given in WO 2004/047833 A1 or WO 2007/062957 A1:

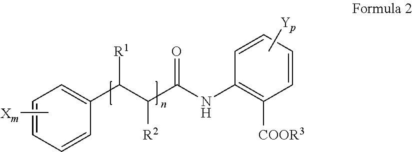

where m=0, 1, 2 or 3, p=0, 1 or 2, and n=0, 1 or 2,
with the proviso that if n=1 or 2, then p+m>0,
and if n=1 or 2, then R¹ and R², in respective pairs, respectively denote H or together denote another chemical bond (as for example in cinnamic acid derivatives),
and if m=1, 2 or 3, then each X independently denotes OH, Oalkyl or Oacyl,
and if p=1 or 2, then each Y independently denotes OH, Oalkyl or Oacyl,
and if p+m>0, then at least one of X and Y is selected from the group consisting of OH and Oacyl,
and where R³ is —H or an alkyl (in particular —CH₃, or other straight-chain or branched alkyl chains with 2 to 30 C atoms; in this context, R³ is also —H for the corresponding pharmaceutically acceptable salts).

Particularly preferred compounds of Formula 2 according to the invention are those in which:

n=1 or 2 and p+m>0; and/or
p+m>0 and X or Y at least one of X and Y is selected from the group consisting of OH and Oalkyl.

Particularly preferably, a compound of Formula 2 is used in which n=1 and p+m>2, with the proviso that at least two of X and Y are together selected from the group comprising OH and Oalkyl.

It is also preferable to use a compound of Formula 2 in which n=1 and m=1, 2 or 3, with the proviso that at least one X is selected from the group comprising OH and Oalkyl, and/or P=1 or 2, with the proviso that at least one Y is selected from the group comprising OH and Oalkyl.

If n has the value 1, then R¹ and R² are each preferably H, although it is also possible for R¹ and R² together to be another chemical bond.

With regard to the definition of Formula 2 and the specific avenanthramide compounds disclosed in WO 2004/047833 A1 or WO 2007/062957 A1, the corresponding disclosure in said documents is hereby incorporated by reference.

The avenanthramide analogue compound of Formula 2 is preferably selected from the group consisting of:

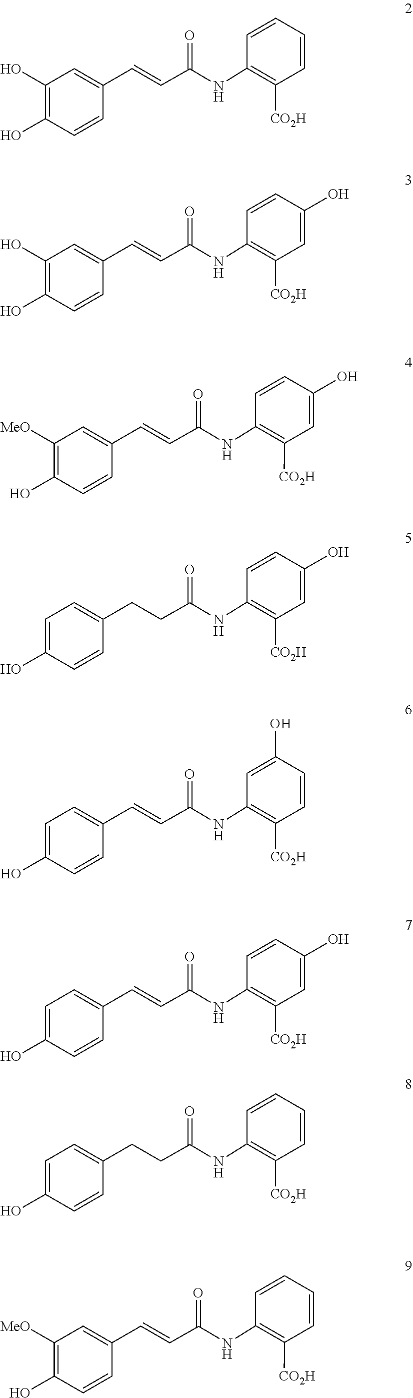

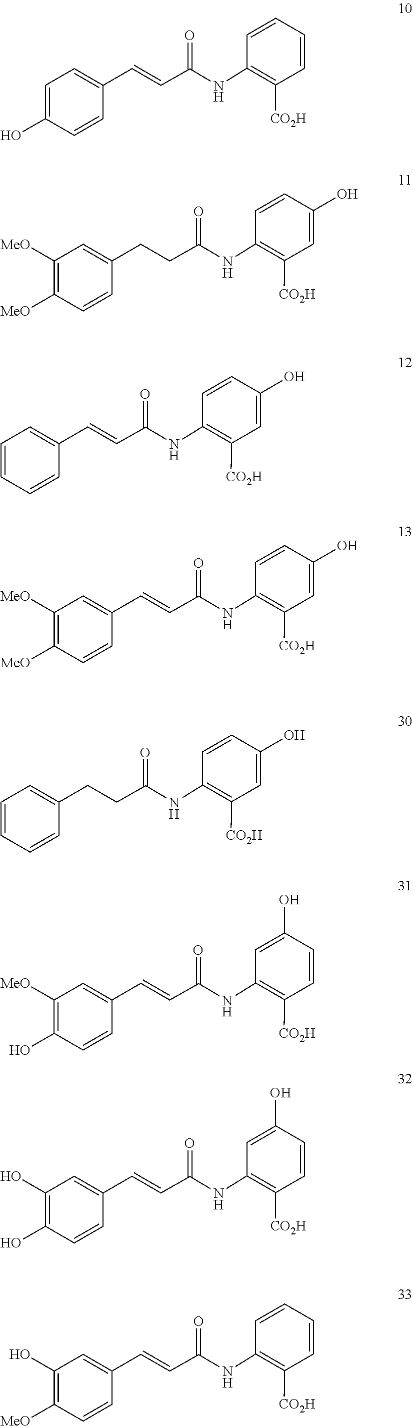

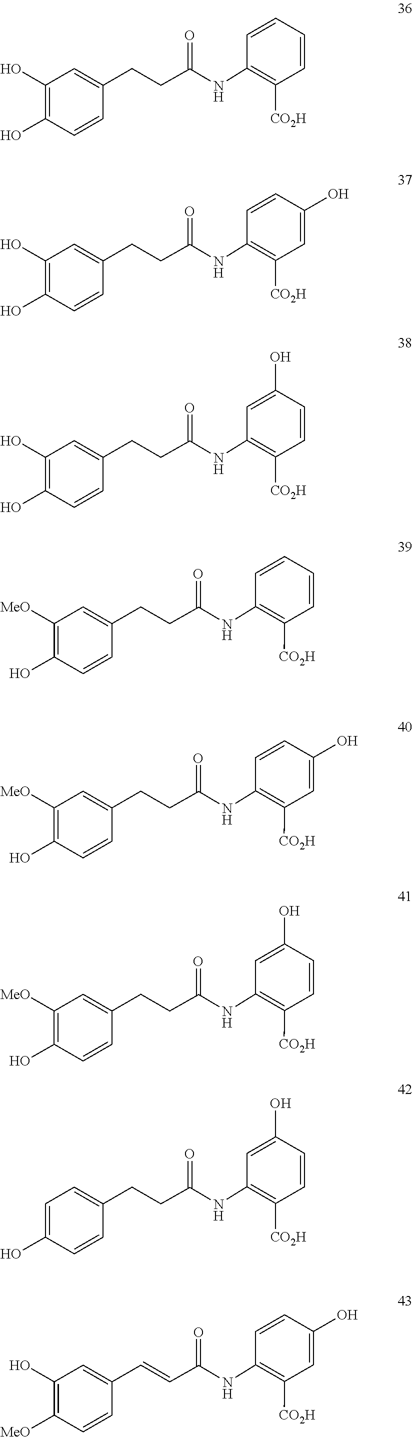

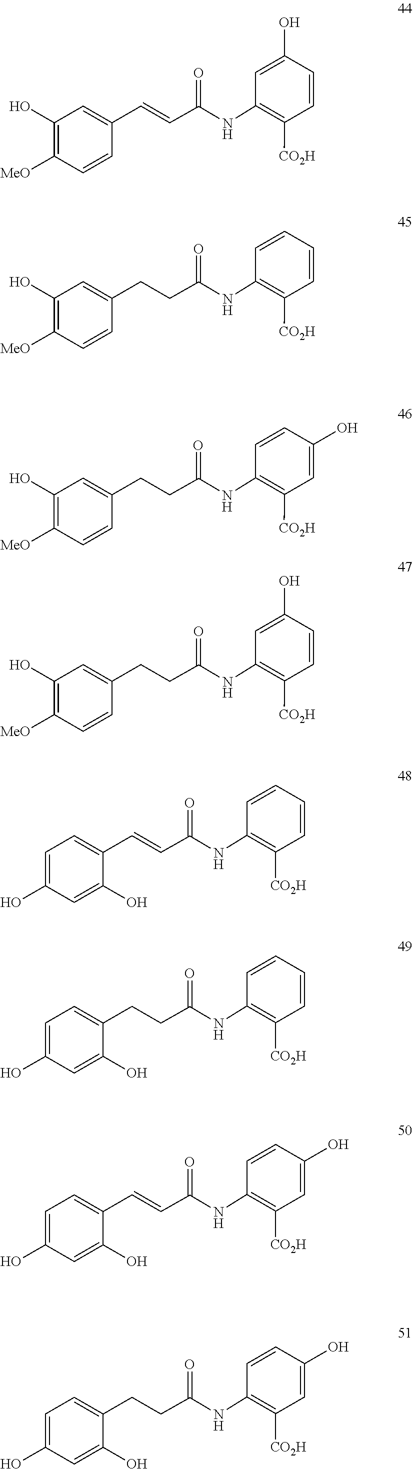

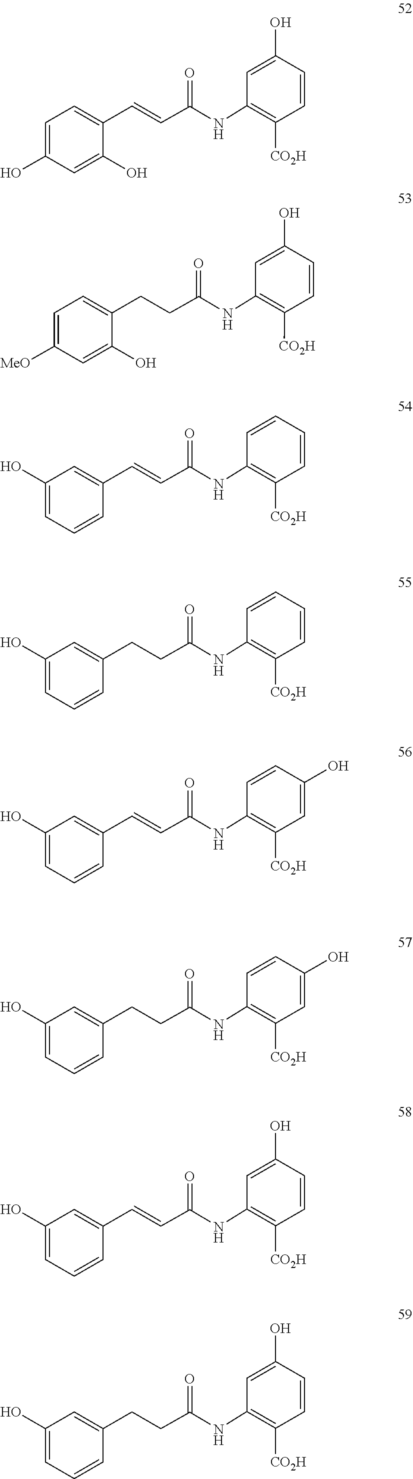

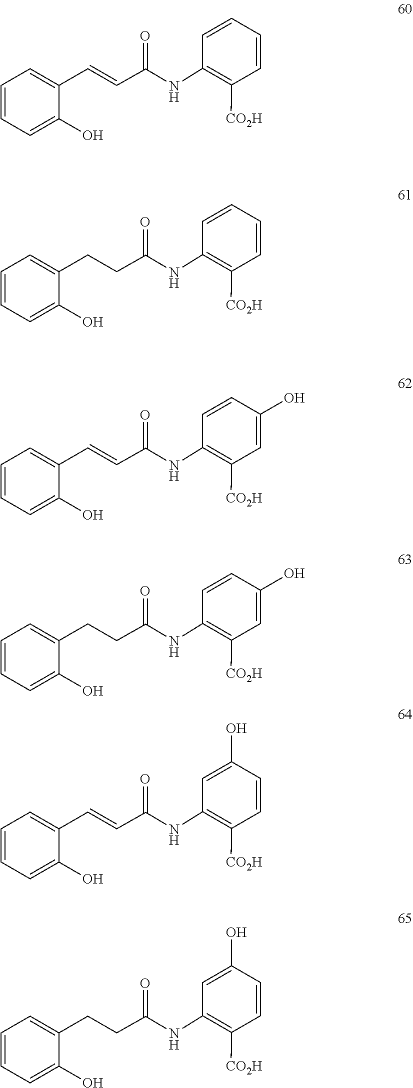 [SMILES: O=C(/C=C/c1ccccc1O)Nc1ccccc1C(=O)O ; O=C(CCc1ccccc1O)Nc1ccccc1C(=O)O ; O=C(/C=C/c1ccccc1O)Nc1ccc(O)cc1C(=O)O ; O=C(CCc1ccccc1O)Nc1ccc(O)cc1C(=O)O ; O=C(/C=C/c1ccccc1O)Nc1cc(O)ccc1C(=O)O ; O=C(CCc1ccccc1O)Nc1cc(O)ccc1C(=O)O]

The above illustrations relate essentially to compounds of Formula 2 in which n=1.

However, the use of compounds of Formula 2 in which n=0 is also frequently preferred, in which case it preferably holds that m+p=0, or m+p>1 or 2, with the proviso that at least two of the substituents X and Y are selected from the group comprising OH and Oalkyl.

It is particularly preferable to use compounds of Formula 2 (where n=0) selected from the group comprising:

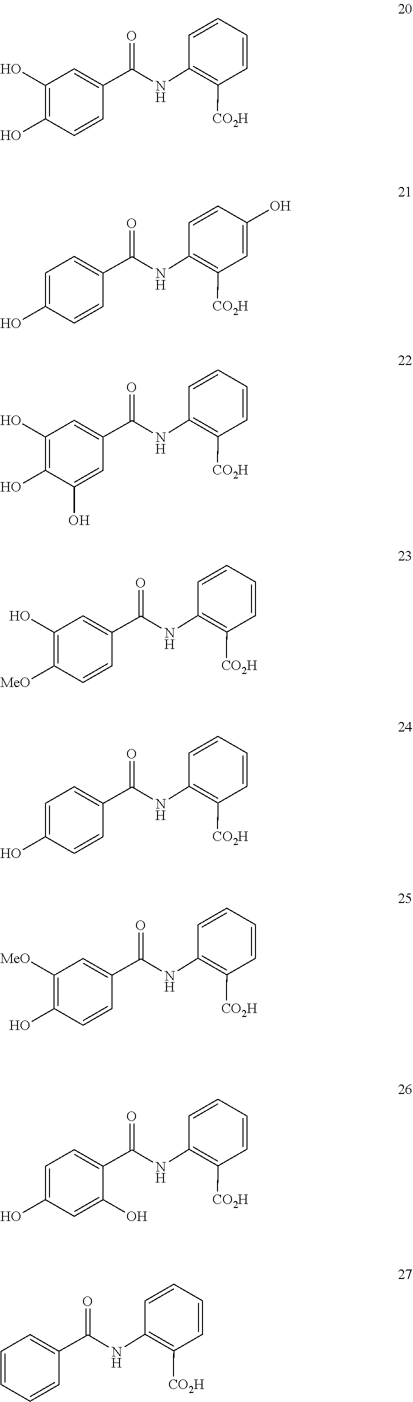

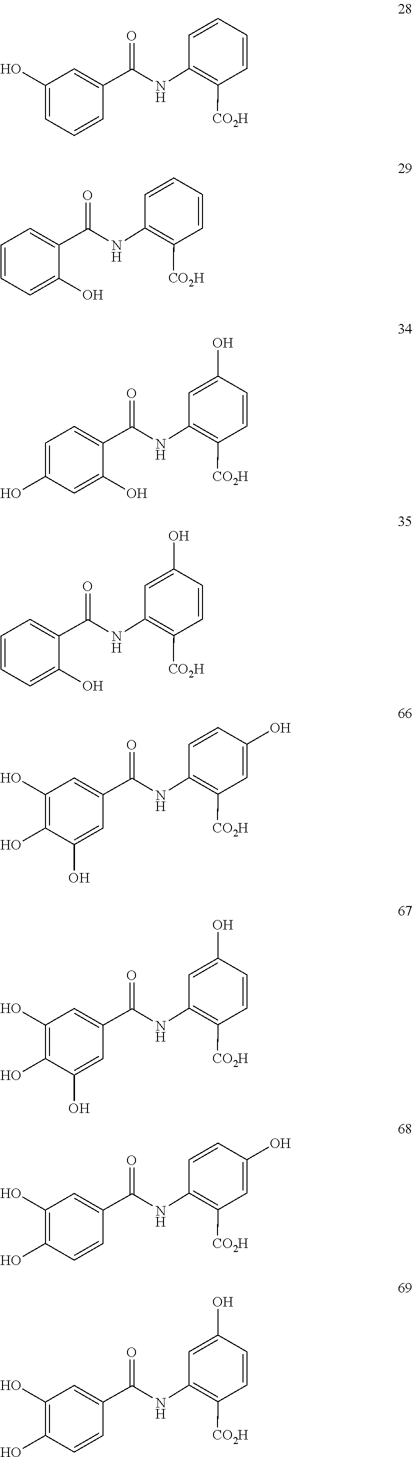

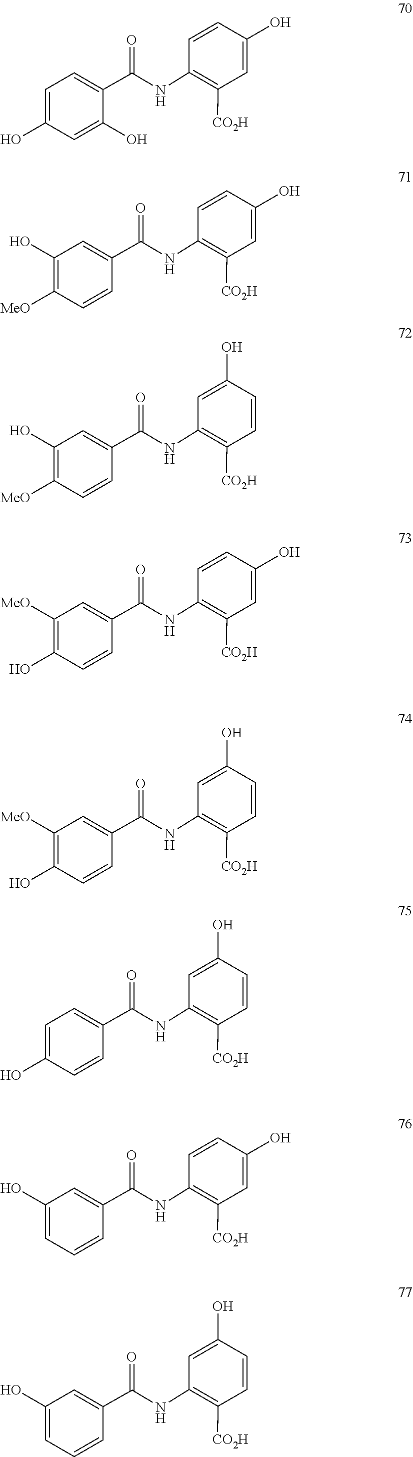

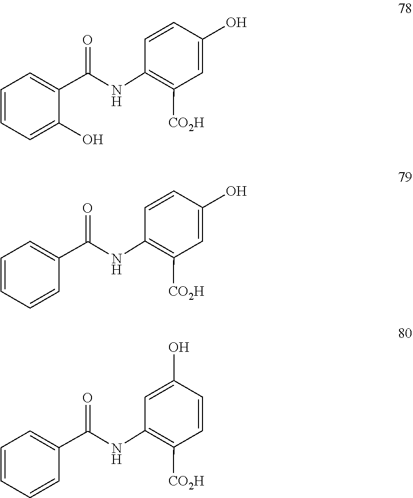 [SMILES: O=C(Nc1ccc(O)cc1C(=O)O)c1ccccc1O ; O=C(Nc1ccc(O)cc1C(=O)O)c1ccccc1 ; O=C(Nc1cc(O)ccc1C(=O)O)c1ccccc1]

From the above avenanthramide analogue compounds No. 8 (dihydroavenanthramide D) and No. 27 are particularly preferred.

Besides the above natural occurring avenanthramides and non-natural occurring avenanthramides analogues, novel avenanthramide analogues have been produced in recombinant yeast, including N-(4′-hydroxycinnamoyl)-3-hydroxyanthranilic acid (YAvn I) and N-(3′-4′-dihydroxycinnamoyl)-3-hydroxyanthranilic acid (YAvn II), which were generated by engineering a Saccharomyces cerevisiae strain with two plant genes (4cl-2 from tobacco and hct from globe artichoke) encoding key proteins involved in the biosynthesis of phenolic esters. Remarkably, YAvn I and YAvn II share structural similarities with Avn A and Avn C, respectively.

Avenanthramide L or the naturally occurring analogue avenanthramide compounds other than avenanthramide L, such as avenanthramides A, B, C, G, H, K etc., represented by the general Formula 1 and specified in Table 1 above, or the non-naturally occurring analogue avenanthramide compounds, represented by the general Formula 2 above, (hereinafter in general designated as “analogues” or “analogue avenanthramide compounds”), and used in accordance with the present invention, are less well studied and described. De Bruijn et al., Food Chemistry 2019, 277, 682-690 identified several by their typical LC-MS fragmentation pattern in oat seedings.

Within the context of the present invention, the term “avenanthramide L” means the compound avenanthramide L (non-Collins abbreviation (also called avenanthramide O (Collins abbreviation) or 2pd) with the CAS number 172549-38-1 itself, represented by the general Formula 1 and defined in Table 1.

Avenanthramide L and the naturally occurring analogue avenanthramide compounds other than avenanthramide L, represented by the general Formula 1 and specified in Table 1 above (hereinafter designated as naturally occurring analogue avenanthramide compounds), are naturally found in and can be isolated and purified from oats. The two main species of oats are Avena sativa L. and Avena nuda L. (synonyms include Avena sativa subsp. nuda (L.) after Gillet & Magne, and Avena sativa var. nuda (L.) after Körn). A. sativa is also known as common or husked oat. A. nuda is known as naked or huskless oat because the husk is removed when the crop is harvested. Oats can be processed and separated into constituent fractions including oat grains, wherein they appear to be most concentrated in the peripheral regions, husks, trichomes or straw.

In a another version, avenanthramide L and the naturally occurring avenanthramide compounds are isolated from oats, Avena sativa L. or Avena nuda L., infected by pathogens or treated with elicitors, in particular inoculated with Puccinia coronata f. sp. avenae.

Avenanthramide L and the naturally occurring analogue avenanthramide compounds isolated from natural sources can alternatively also be produced by organic synthesis. Methods of synthesis known in the art are illustrated for example in U.S. Pat. Nos. 6,096,770 and 6,127,392, Japanese Patent No. J60019 754 A and Hungarian Patent No. HU 200 996 B.

Said synthetic prepared avenanthramide substances are identical to the corresponding naturally occurring avenanthramide compounds as extracted from oats.

Apart from the natural occurring avenanthramide L and natural occurring analogue avenanthramide compounds isolated from oats, non-naturally occurring analogue avenanthramide compounds, represented by the general Formula 2 and as defined above (hereinafter designated as non-naturally occurring analogue avenanthramide compounds) are artificially produced by organic synthesis methodologies, according to steps known in the literature, such as for example those given in WO 2004/047833 A1 or WO 2007/062957 A1, the corresponding disclosure relating to the avenanthramide L compounds and their analogues in said documents is hereby incorporated by reference.

The term “avenanthramide L” or “analogue avenanthramide compound” is intended to also include their various isomers that exist, notably the naturally occurring trans-isomers as well as the cis-isomers, induced e.g. by photoisomerization due to light exposure.

In a preferred variant of the present invention, natural avenanthramide L enriched, isolated and purified from oats is used in accordance with the present invention.

The avenanthramide L or the naturally occurring avenanthramide compounds other than avenanthramide L are obtained and isolated from the plant of the genus Avena by extraction, in particular from any oat species, fresh or dried, or parts thereof, such as milled grains, non-milled grains, husks, trichomes or oat straw of the oat species Avena sativa or Avena nuda.

In a preferred variant, the starting material for the oat extract is milled or non-milled grains of the species Avena sativa or Avena nuda or oat straw.

The extracting solvent (extractant) for favourably extracting avenanthramide L or the naturally occurring avenanthramide compounds other than avenanthramide L is selected from the group consisting of mixtures of water and an organic solvent, wherein the organic solvent is preferably a solvent suitable for foodstuffs or cosmetic or pharmaceutical preparations. It goes without saying that such solvents need be suitable for and compatible with the preparation of foods, cosmetics or pharmaceutical preparations.

In a more preferred variant, the extracting solvent comprises a mixture of water and an alcohol or acetone. The alcohol is preferably selected from the group consisting of methanol, ethanol, n-propanol, isopropanol, n-butanol, isobutanol, t-butanol and mixtures, i.e. combinations, thereof. The most preferred extracting solvents (extractant) for the extraction step of the present invention are methanol, ethanol, n-propanol, isopropanol or acetone or any mixtures respective combinations of said solvents, each in mixture with water. The use of pure organic solvents is not advantageous, due to the co-extraction of triglycerides.

The mixing ratio of water to the organic solvent, preferably water to the alcohol or water to acetone, in the extracting solvent is in a range of 10:90 to 90:10 (v/v), preferably in a range of 20:80 to 80:20 (v/v) and most preferably in a range of 30:70 to 70:30 (v/v), based in each case on the resulting extracting solvent.

Particularly preferred extracting solvents (extractants) are: methanol/water (3 7), methanol/water (1:1), methanol/water (7:3), ethanol/water (3:7), ethanol/water (1:1), ethanol/water (1:4), ethanol/water (7:3), isopropanol/water (3:7), isopropanol/water (1:1), isopropanol/water (7:3), aceton/water (3:7), aceton/water (1:1), aceton/water (7:3).

From said extracting mixtures (extractants) methanol/water (1:1), methanol/water (7:3), ethanol/water (1:1), ethanol/water (1:4), isopropanol/water (3:7), isopropanol/water (1:1), isopropanol/water (7:3), aceton/water (3:7), aceton/water (1:1) and aceton/water (7:3) are particularly advantageous, since the extraction with these extractants results in an extract with high avenanthramide L content (see Table 10). The yield of avenanthramide L with these extractants is >150 ppm, more preferably >190 ppm and most preferably >200 ppm.

In order to improve the extraction yield, the oat source is extracted at a temperature ranging from 30 to 80° C., preferably from 40 to 70° C. and more preferably from 50 to 60° C. The extraction yield for milled oat grains increases with increasing temperatures between 40 and 70° C. Extracting from milled oats gives the best results in terms of yield and avenanthramide L content at temperatures between 50 and 60° C., which is therefore preferred.

Alternatively to the natural or synthetic single avenanthramide L compound, an oat extract comprising avenanthramide L may also be used in accordance with the invention. Within the contect of the present invention, the term “oat extract” is generally meant to encompass a compound or mixture of compounds obtained from oats.

Such extract comprising avenanthramide L or encompassing a mixture of avenanthramide L and naturally occurring analogue avenanthramide compounds other than avenanthramide L as described above, are obtained by extraction (such as maceration, percolation, extraction by use of soxhlet, microwave or ultrasound) with water, an alcohol, acetone or mixtures thereof or by subcritical fluid extraction with these solvents or mixtures thereof. They are preferably extracted using various solvent compositions such as pure methanol, ethanol, n-propanol, isopropanol, n-butanol, isobutanol, t-butanol and mixtures, i.e. combinations, thereof or said solvents in mixture with water. Extraction procedures were achieved over different times at room temperature or under controlled heating, such as naked oats, 50% aqueous ethanol [Tong L. et al., Journal of Integrative Agriculture 2014, 13, 1809]. Maliarova, M. et al., Journal of the Brazilian Chemical Society 2015, 26(11), 2369-2378 compared the efficiency of methanol, ethanol and isopropanol on the extraction of Avns from naked oat bran. The optimum conditions for the highest yield of Avns are a methanol concentration of 70%, an extraction temperature of 55° C. and an extraction time of 165 minutes.

The extract is obtained from the plant of the genus Avena, in particular from any oat species, fresh or dried, or parts thereof, such as milled grains, non-milled grains, husks, trichomes or oat straw of the oat species Avena sativa or Avena nuda. Starting product for the extraction can also be oat grain residues from oat oil production.

In a preferred variant, the starting material for the oat extract is milled or non-milled grains of the species Avena sativa or Avena nuda or oat straw.

The extracting solvent (extractant) for favourably extracting the avenanthramide L and the naturally occurring analogue avenanthramide compounds is selected from the group consisting of mixtures of water and an organic solvent, wherein the organic solvent is preferably a solvent suitable for foodstuffs or cosmetic or pharmaceutical preparations. It goes without saying that such solvents need be suitable for and compatible with the preparation of foods, cosmetics or pharmaceutical preparations.

In a more preferred variant, the extracting solvent comprises a mixture of water and an alcohol or acetone. The alcohol is preferably selected from the group consisting of methanol, ethanol, n-propanol, isopropanol, n-butanol, isobutanol, t-butanol and mixtures, i.e. combinations, thereof. The most preferred extracting solvents (extractant) for the extraction step of the present invention are methanol, ethanol, n-propanol, isopropanol or acetone or any mixtures respective combinations of said solvents, each in mixture with water. The use of pure organic solvents is not advantageous, due to the co-extraction of triglycerides.

The mixing ratio of water to the organic solvent, preferably water to the alcohol or water to acetone, in the extracting solvent is in a range of 10:90 to 90:10 (v/v), preferably in a range of 20:80 to 80:20 (v/v) and most preferably in a range of 30:70 to 70:30 (v/v), based in each case on the resulting extracting solvent.

Particularly preferred extracting solvents (extractants) are: methanol/water (3 7), methanol/water (1:1), methanol/water (7:3), ethanol/water (3:7), ethanol/water (1:1), ethanol/water (1:4), ethanol/water (7:3), isopropanol/water (3:7), isopropanol/water (1:1), isopropanol/water (7:3), acetone/water (3:7), acetone/water (1:1), acetone/water (7:3).

From said extracting mixtures (extractants) methanol/water (1:1), methanol/water (7:3), ethanol/water (1:1), ethanol/water (1:4), isopropanol/water (3:7), isopropanol/water (1:1), isopropanol/water (7:3), acetone/water (3:7), acetone/water (1:1) and acetone/water (7:3) are particularly advantageous, since the extraction with these extractants results in an extract with high avenanthramide L content (see Table 10). The yield of avenanthramide L with these extractants is >150 ppm, more preferably >190 ppm and most preferably >200 ppm.

In order to improve the extraction yield, the oat source is extracted at a temperature ranging from 30 to 80° C., preferably from 40 to 70° C. and more preferably from 50 to 60° C. The extraction yield for milled oat grains increases with increasing temperatures between 40 and 70° C. Extracting from milled oats gives the best results in terms of yield and avenanthramide(s) content, in particular avenanthramide L content, at temperatures between 50 and 60° C., which is therefore preferred.

Altering the composition of the solvent can change the extract selectivity of the avenanthramide substances to be extracted, and thus the composition, thereby enhancing or reducing its biological activity.

In a preferred variant of the present invention the oat extract comprises at least avenanthramide L or comprises at least avenanthramide L and one or more analogue avenanthramide compound thereof as described and defined above.

In another preferred variant of the present invention, avenanthramide L or the oat extract comprising avenanthramide L may further be used in combination with one, two, three or even more naturally occurring analogue avenanthramide compound(s) other than avenanthramide L and selected from the group consisting of avenanthramides represented by the general Formula 1 or specified in Table 1 as described and defined above. The resulting mixtures of avenanthramides can thus include any possible combinations of avenanthramide L and one or more analogue avenanthramide compound(s) other than avenanthramide L, as specified and defined above in Table 1.

In another preferred variant of the present invention, avenanthramide L or the oat extract comprising avenanthramide L may further be used in combination with one, two, three or even more non-naturally occurring analogue avenanthramide compound(s) other than avenanthramide L and selected from the group consisting of avenanthramides as represented by the above general Formula 2 as described and defined above. The resulting mixtures of avenanthramides can thus include any possible combinations of avenanthramide L and one or more analogue avenanthramide compound(s) other than avenanthramide L, as represented by the above general Formula 2.

Preferably, the avenanthramide L or the oat extract comprising avenanthramide L obtained from oats and used in accordance with the present invention may thus further be used in combination with at least one further analogue avenanthramide selected from the group consisting of avenanthramides A, B, C, G, H, K and R. Within the scope of the present invention, any combinations of avenanthramide L or oat extract comprising avenanthramide L in combination with one, two, three or even more other naturally occurring analogue avenanthramide compound(s), selected from the group consisting of A, B, C, G, H, K and R are encompassed.

In a preferred variant, the avenanthramide L or the oat extract comprising avenanthramide L can comprise the following combinations of avenanthramides: Avns L and A; Avns L and B; Avns L and C; Avns L and G; Avns L and H; Avns L and K; Avns L and R; Avns L, A, B; Avns L, A, C; Avns L, A, G; Avns L, A, H; Avns L, A, K; Avns L, A, R, Avns L, B, C; Avns L, B, G; Avns L, B, H; Avns L, B, K; Avns L, B, R; Avns L, C, G; Avns L, C, H; Avns L, C, K; Avns L, C, R; Avns L, G, H; Avns L, G, K; Avns L, G, R; Avns L, H, K; Avns L, H, R; Avns L, K, R; Avns L, A, B, C; Avns L, A, B, G; Avns L, A, B, C, H; Avns L, A, B, C, K; Avns L, A, B, C, R; Avns L, A, C, G; Avns L, A, C, H; Avns L, B, C, G; Avns L, B, C, H; Avns L, B, C, K; Avns L, B, C, R; Avns L, C, G, H; Avns L, C, G, K; Avns L, C, G, R; Avns L, G, H, K; Avns L, G, H, R and Avns L, H, K, R.

In addition, the avenanthramide L or the oat extract comprising avenanthramide L can also comprise avenanthramides other than the avenanthramides A, B, C, G, H, K, L and R, such as avenanthramides D, E, F U, X, Y (also termed 2), AA, CC or 00 as specified in Table 1.

Particularly preferred combinations are Avns L and A; Avns L and B; Avns L and C; Avns L and G; Avns L and H; Avns L and K; and Avns L and R. The most preferred mixtures of avenanthramides are however Avns L and A and Anvs L in combination with A/B/C. Very particularly preferred is a combination of Avn L and Avn A due to its synergistic effect as it is demonstrated in Example 7.

Altering the composition of the solvent can change the extract selectivity of the avenanthramide substances to be extracted, and thus the composition of the preparation, thereby enhancing or reducing its biological activity.

In a further preferred variant, the avenanthramide L or the oat extract comprising avenanthramide L can comprise the following combinations of avenanthramides: Avn L and compound No. 8 (dihydroavenanthramide D) or Avn L and compound No. 27.

Surprisingly, it turns out that avenanthramide L or an oat extract comprising avenanthramide L exhibits highly interesting biological benefits, such as anti-inflammatory, antioxidant, anti-itching, anti-irritant and anti-atherogenic activities, and are thus beneficial agents for skin protection and in the prevention and/or treatment of dermatoses. In particular, it turns out that avenanthramide L or an oat extract comprising avenanthramide L is an effective agent in the prevention and/or treatment of dermatoses, in particular of dermatological or keratological disorders having a barrier related, inflammatory, immunoallergic, atherogenic, xerotic or hyperproliferative type.

### Use of Avenanthramide L or an Oat Extract Comprising Avenanthramide L as an Antagonist of the Neurokinin-1 Receptor NK1R

According to the first aspect, the invention pertains to the use of avenanthramide L or an oat extract comprising avenanthramide L as an antagonist of the neurokinin-1 receptor NK1R.

Accordingly, the present invention relates to a method for inhibiting the neurokinin-1 receptor NK1R in a subject in need thereof, wherein the method comprises administering to the subject avenanthramide L or an oat extract comprising avenanthramide L in an amount which is sufficient for inhibiting the neurokinin-1 receptor NK1R in the subject.

Surprisingly, it turns out that avenanthramide L or an oat extract comprising avenanthramide L has the ability to antagonise the binding of SP at the neurokinin-1 receptor NK1R.

It is known that substance P (SP) plays a major pathogenic role, as it is an important mediator of inflammation. SP is a member of the tachykinin family of peptides and acts as a neurotransmitter or modulator in the mammalian peripheral and central nervous system (CNS). SP is produced and secreted by nerve fibres and binds to the neurokinin-1 receptor NK1R. The neurokinin-1 receptor NK1R is a tachykinin receptor and belongs to the G protein-coupled receptor family, known to activate signal transduction pathways within the cell. In addition to their production from neurons, the SP and its NK1 receptor complex are well documented as being expressed in different immune cell types, in particular on multiple skin cell types involved in the initiation and transmission of itching, including keratinocytes, fibroblasts and mast cells.

In particular, the role of SP in keratinocytes, fibroblasts and mast cells appears to be predominantly related to the induction of inflammation associated with erythema, wheals and pruritis (itching).

As increasingly documented, the SP-NK1 receptor system induces or modulates many aspects of the immune response. Activation of the neurokinin-1 receptor NK1R can induce a phospholipase C (PLC)/inositol-1,4,5-triphosphate (IP3)-dependent Ca²⁺-signalling pathway resulting in inflammation due to the production of pro-inflammatory cytokines such as interleukin. Both receptors are, for example, involved in the induction and maintenance of pruritus. Preventing the actions of SP through the use of NK1 receptor antagonists is emerging as a promising therapeutic approach for the treatment of skin disorders, in particular skin disorders with an inflammatory component.

The ability of avenanthramide L to inhibit the neurokinin-1 receptor NK1R may be demonstrated using assays of human recombinant CHO cells, as in Example 1.

Surprisingly, avenanthramide L is about twice as active as avenanthramide A at each of the different concentrations 100, 10, 1 and 0.1 ppm. Avenanthramide L is surprisingly also more active than the known synthetic neurokinin-1 receptor NK1R antagonist dihydroavenanthramide D.

Avenanthramide C is approximately twice as active as avenanthramide L, but is highly unstable, whereas avenanthramide L is significantly less degradable by the action of oxygen and temperature exposure, as demonstrated in Example 2 below.

The use of avenanthramide L in accordance with the present invention exhibits marked activity against the neurokinin-1 receptor NK1R as described in the foregoing test and is considered a promising avenue for the treatment of diseases in which the neurokinin-1 receptor NK1R is implicated, in particular as a cosmetic for skin care, scalp care, hair car, nail care and/or for use in the prevention and/or treatment of skin conditions, intolerant and sensitive skin, skin irritation, skin reddening, wheals, pruritis (itching), skin aging, wrinkle formation, loss of skin volume, loss of skin elasticity, pigment spots, pigment abnormalities, dry skin, i.e. for moisturising the skin or as a medicament in the prevention and/or treatment of dermatological or keratological diseases, in particular dermatological or keratological diseases having a barrier related, inflammatory, immunoallergic, atherogenic, xerotic or hyperproliferative component.

Avenanthramide L is also significantly less degradable than avenanthramides A and C, as demonstrated in Example 2.

### Use of Avenanthramide L or an Oat Extract Comprising Avenanthramide L for Inducing Expression of Small Heat Shock Proteins or for Inducing Expression of CD44

According to the second aspect, the invention relates to the use of avenanthramide L or an oat extract comprising avenanthramide L for inducing the expression and/or gene expression of small heat shock proteins or for inducing the expression and/or gene expression of CD44.

Accordingly, the present invention relates to a method for inducing the expression and/or gene expression of small heat shock proteins or for inducing the expression and/or gene expression of CD44 in a subject in need thereof, wherein the method comprises administering the subject with avenanthramide L or an oat extract comprising avenanthramide L in an amount which is sufficient for inducing the expression and/or gene expression of small heat shock proteins or for inducing the expression and/or gene expression of CD44 in the subject.

Surprisingly, it turns out that avenanthramide L or an oat extract comprising avenanthramide L has the ability to induce the expression and/or gene expression of small heat shock proteins (sHSPs).

Organisms and cells respond to various stress conditions such as environmental, metabolic or pathophysiological stress by selectively upregulating the expression of a group of proteins called heat shock proteins (HSPs).

HSPs are molecular chaperones, stabilising new proteins to ensure correct folding or helping to refold proteins damaged by the cell stress, thus preventing apoptosis. Small heat shock proteins (sHSPs) are a ubiquitous and ancient family of ATP-independent molecular chaperones with low molecular mass (12-43 kDa). The HSPs are identified by their increased expression after a heat shock (usually one hour or more after exposure to temperatures of 3 to 5° C. above normal temperatures). The dramatic upregulation of the heat shock proteins is a key part of the heat shock response and is induced primarily by heat shock factor (HSF).

The assumption that HSPs protect cells from heat damage is supported by the following facts: 1) HSP expression occurs exactly in parallel with the development of and drop in thermotolerance (resistance to heat-induced inactivation); 2) mutation or inactivation of the HSPs impairs a cell's ability to survive at high temperatures; 3) overexpression of HSPs can often improve a cell's ability to resist high temperatures. Inducing heat shock proteins using Avn L has not previously been described.

These proteins have been classified into six major families, based on their molecular masses, namely HSP100, HSP90, HSP70, HSP60, HSP40 and small heat shock proteins (sHSPs). sHSPs have subunit molecular masses of 12 to 43 kDa. Examples of small heat shock proteins include HSPB1, HSPB2 and HSPB3 (HSP27), HSPB4 (αA-crystallin), HSPB5 (αB-crystallin), HSPB6 (HSP20) and HSPB8 (HSP22).

Extensive research has demonstrated that a majority of sHSPs, and also αA-crystallin, can act as ATP-independent molecular chaperones by binding denaturing proteins and thereby protecting cells from damage due to irreversible protein aggregation, in particular under conditions of stress that lead to unfolding of cellular proteins. In addition to molecular chaperone-like activity in preventing aggregation of proteins/peptides, sHSPs such as HSP27 and αB-crystallin are also involved in diverse cellular functions such as stress tolerance, protein folding, protein degradation, maintaining cytoskeletal integrity, cell death, differentiation, cell cycle and signal transduction and development. Members of the sHSP family exhibit cardio and neuroprotection, potent anti-apoptotic activity, pro-angiogenic property and anti-inflammatory properties involving interactions. Aside from this, small heat shock proteins can also stimulate immune receptors and are important in the proper folding of proteins involved in pro-inflammatory signalling pathways.

Human sHSPs exhibit highly differing features with regard to their heat-induced expression, tissue and intracellular localisation, structure, substrate preference and function. Due to these differences, human sHSPs exhibit different abilities with respect to protecting against acute and different types of chronic (disease-related) stress.

sHSP27 (HSPB1, HSPB2, HSPB3) and αB-crystallin (CRYAB/HSPB5), as specified above, can act as an ATP-independent molecular chaperone which protects cells from damage due to irreversible protein aggregation, in particular under conditions of stress. Usually sHSPs stabilize early unfolding intermediates of aggregation-prone proteins which arise during diverse stress conditions. HSP27 (HSPB2) can be found in various cells and tissues also without prior stress stimulation e.g. in epidermal skin. It provides its chaperone function as large oligomer complex. The inducibility of HSP27 decreases with age. In addition to its chaperone function, HSP27 is linked to skin barrier: its expression correlates with keratinocyte differentiation and increases continuously from the basal layer to the stratum granulosum. Keratinocyte differentiation leads to the formation of the cornified layer of the skin which is important for the formation of a competent epidermal barrier. Loss of HSP27 is associated with hyperkeratinization and misprocessing of profilaggrin. αB-Crystallin (HspB5) is constitutively expressed in many tissues and has anti-apoptotic properties and chaperone activity. It can form oligomers with other HSPs, namely with HSP27. HSP27 and αB-crystallin (CRYAB) are localised in intact skin in the stratum corneum and stratum spinosum.

The ability of avenanthramide L to upregulate the small heat shock proteins HSP27 (HSPB2) and αB-crystallin (CRYAB) may be demonstrated by Example 3 below.

The results show surprisingly, that avenanthramide L at 100 μM upregulates the small heat shock proteins HSP27 (HSPB2) and αB-crystallin (CRYAB) but has no effect on the large heat shock proteins HSP90AA1 and HSP90AB1. In addition, avenanthramide L upregulates the small heat shock proteins more effectively than avenanthramide A when tested at the same test concentration.

Thus, in a preferred variant of the second aspect of the present invention, the small heat shock proteins upregulated by avenanthramide L or an oat extract comprising avenanthramide L is HSP27 or αB-crystallin (CRYAB).

The use of avenanthramide L in accordance with the present invention exhibits marked activity in the aforementioned test and is thus considered to be useful as a physiological response for mediating repair mechanisms, reducing cellular damage and in the formation of a competent epidermal barrier. In addition, the induction of the expression of small heat shock proteins may be an important mechanism for protecting human skin, hair and nails from environmental, metabolic or pathophysiological stress.

It also turns out that avenanthramide L or an oat extract preparation comprising avenanthramide L has the ability to induce the expression and/or gene expression of CD44.

CD44 is the most well-studied hyaluronic acid (HA) receptor and the predominant receptor for HA on the cell surface of keratinocytes. Matrix HA is the major glycosaminoglycan in the extracellular matrix (ECM) of most mammalian tissues, including epidermis and dermis, and HA has been implicated in several skin epidermal functions. Down-regulation of CD44 in cultured keratinocytes (using CD44 siRNA) also significantly inhibits HA mediated keratinocyte differentiation and lipid synthesis [L. Y. Bourguignon et al., J. Invest. Dermatol. 2006, 1356-1365].

CD44 generally upregulates pro-proliferative and migratory effects of cells in tissues that contain abundant HA. HA levels and/or the interactions of HA and CD44 are able to regulate cellular differentiation (e.g., the cornification of epidermal keratinocytes and the differentiation of fibroblasts into myofibroblasts). During normal tissue homeostasis, hyaluronan synthesis and degradation in the epidermis are active, but balanced. However, whenever this homeostasis is disturbed with insults such as wounding, barrier disruption, or UVB radiation, epidermal hyaluronan content is rapidly increased. An increased expression of CD44 which is seen after epidermal insults closely correlates with hyaluronan accumulation. HA acting together with its receptor CD44 supports cell survival and stimulated HA synthesis through upregulated HA synthase expression is an inherent feature of the keratinocyte activation triggered by tissue trauma, and presumably important for a proper healing response. CD44 also appears to have a role in limiting inflammatory responses, which has also been shown in inflammation models.

Aged epidermis is often characterized by abnormal barrier function and, impaired lipid synthesis. Epidermal dysfunction and abnormal keratinocyte activities in aged skin often lead to debilitating clinical consequences (e.g. epidermal thinning (atrophy), barrier dysfunction, xerosis/xerotic eczema, delayed wound healing, and inflammation). Recent studies have revealed that abnormal HA metabolism may be involved in the changes associated with keratinocyte activities, permeability barrier homeostasis, and wound healing during skin aging.

The ability of avenanthramide L to upregulate the expression of CD44 may be demonstrated by Example 4 below.

The results show, surprisingly, that avenanthramide L at 100 μM upregulates the expression of CD44, while avenanthramide A has no effect at the same test concentration.

The use of avenanthramide L in accordance with the present invention exhibits marked activity in the aforementioned test and is thus considered to be useful as a physiological response for HA/CD44 mediated activities such as cellular differentiation, proliferation and migration, barrier homeostasis, skin hydration and wound healing. In addition, the induction of the expression of CD44 may be an important mechanism for protecting human skin, hair and nails from environmental, metabolic or pathophysiological stress.

### Use of Avenanthramide L or an Oat Extract Comprising Avenanthramide L as an Antioxidant or for Inducing Expression of BLVRB

According to the third aspect, the invention relates to the use of avenanthramide L or an oat extract comprising avenanthramide L as an antioxidant or for inducing the expression of BLVRB.

Accordingly, the present invention relates to a method for inhibiting ROS formation in a subject in need thereof, wherein the method comprises administering the subject with avenanthramide L or an oat extract comprising avenanthramide L in an amount which is sufficient for inhibiting ROS formation in the subject.

The term “antioxidant” as used in this document refers to a substance or composition which, when present in a mixture or structure containing an oxidisable substrate molecule (such as an oxidisable biological molecule or oxidisable indicator), significantly delays, prevents or even inhibits oxidation of the oxidisable substrate molecule. Antioxidants can act by scavenging biologically important reactive free radicals or other reactive oxygen species or by preventing their formation or by catalytically converting the free radical or other reactive oxygen species into a less reactive species.

Surprisingly, it turns out that avenanthramide L or an oat extract comprising avenanthramide L has a superior radical-scavenging activity and thus a significant antioxidative capacity.

In a biological context, reactive oxygen species (ROS) are formed as a natural by-product of the normal metabolism of oxygen and have important roles in cell signalling and homeostasis. However, at times of environmental stress (for example UV or heat exposure), ROS levels can increase dramatically. Cumulatively, this is known as oxidative stress.

Oxidative stress occurs either when excess ROS are produced in cells, which could overwhelm the normal antioxidant capacity, or when antioxidant defence mechanisms are impaired. Reactive oxygen species (ROS) are chemically reactive chemical species containing oxygen. Examples of ROS include superoxide anions (O₂^{•-}), hydroxyl (OH^{•}), peroxyl (RO₂^{•}) alkoxyl (RO^{•}) radicals, and non-radical compounds such as hydrogen peroxide (H₂O₂), hypochlorous acid (HOCl) and organic peroxides, which can be produced from either endogenous sources (for example mitochondrial electron transport chain, cytochrome P450 monooxygenases, and NADPH oxidases) or exogenous sources (for example pollutants, drugs, xenobiotics and radiation). ROS toxicity affects major cellular components and contributes to significant protein, lipid and DNA damage, inflammation, cell and tissue injury, and apoptosis.

Antioxidants are substances which protect cells from oxidative damage and thereby help in preventing or alleviating several chronic diseases caused by reactive oxygen species (ROS) generation. Several preliminary studies have reported significant antioxidant activity in oat extracts. Several compositions containing oat avenanthramides or derivatives have been described for use in cosmetic, nutraceutical and therapeutic preparations, due to their antioxidant and anti-aging activities. However, the specific component in the extract responsible for this activity was not known. In a study, the three most abundant avenanthramides A, B and C were synthesised and purified, and their antioxidant activity was measured in in vitro systems. All the avenanthramides showed antioxidant activity. The order of antioxidant activity was found to be Avn C>Avn B>Avn A.

There is compelling evidence that oxidative stress plays a major role in the pathogenesis and progression of major human diseases, including inflammatory diseases, and that it is also implicated in aging. It not only directly damages the cellular structures of the skin but also enhances dermal inflammation and weakens the skin barrier function and enables infections by microbial pathogens. According to the free radical theory of aging, oxidative damage initiated by reactive oxygen species (ROS) is a major contributor to the functional decline that is characteristic of aging.

The ability of avenanthramide L to scavenge radicals or inhibit radical formation and its cellular antioxidant activity may be demonstrated by Examples 5 and 6 below.

The ABTS assay measures the relative ability of antioxidants to scavenge the ABTS radical generated in aqueous phase, as compared with a Trolox (water-soluble vitamin E analogue) standard. The green-blue stable radical cationic chromophore 2,2′-azino-bis(3-ethylbenzothiazoline-6-sulphonate) (ABTS^{•+}) is generated by reaction with the ABTS salt using a strong oxidising agent (for example potassium permanganate or potassium persulphate) and has absorption maxima at 414, 645, 734 and 815 nm. The reduction of the blue-green ABTS radical by hydrogen-donating antioxidants is measured by the suppression of its characteristic long wave absorption spectrum.

The results of the ABTS assay show that avenanthramide L exhibits an excellent antioxidative capacity by means of radical-scavenging activity, with similar (at a concentration of 5 μM) or even improved (at a concentration of 10 μM) antioxidant activity as compared to avenanthramide A, as demonstrated in Example 5 below, which makes it beneficial as an antioxidant.

Avenanthramide L has a radical-scavenging activity of at least 40% when used at a concentration of 5 μM as determined using an ABTS assay. In a preferred variant of the present invention, avenanthramide L has a radical-scavenging activity of at least 70% when used at a concentration of 10 μM.

The DCF-DA assay is a fluorometric microplate assay for the detection of oxidative stress by detecting oxidation of 2′,7′-dichlorofluorescein-diacetate (DCF-DA) into the highly fluorescent compound 2′,7′-dichlorofluorescein (DCF) due to the presence of reactive oxygen species (ROS). The DCF-DA assay allows the cellular antioxidant activity of a substance to be determined.

The results of the DCF-DA assay clearly show, surprisingly, that in the cellular system, avenanthramide L exhibits a higher antioxidant activity than avenanthramide A at the same test concentration of 100 μM.

It also turns out that avenanthramide L or an oat extract comprising avenanthramide L has the ability to induce the expression and/or gene expression of BLVRB.

Biliverdin reductase is an enzyme found in all tissues under normal conditions. There are two isozymes, in humans, each encoded by its own gene, biliverdin reductase A (BLVRA) and biliverdin reductase B (BLVRB). Biliverdin reductase converts biliverdin to bilirubin which is a chain-breaking intracellular antioxidant and a scavenger of free radicals. Bilirubin is converted back into biliverdin through the actions of reactive oxygen species (ROS). This cycle allows therefore the neutralization of ROS and the reductase function of biliverdin reductase is therefore considered to be cytoprotective. B. Bai et al. [J. Photochem. Photobiol. B, 2015, 144, 35-41] showed that biliverdin plays a role in prevention of UVB irradiation-induced skin photo-damage mediated by its antioxidant mechanism and cell signal regulatory action.

The ability of avenanthramide L to upregulate the gene expression of BLVRB may be demonstrated by Example 4 below.

The results show, surprisingly, that avenanthramide L at 100 μM upregulates the gene expression of BLVRB, while avenanthramide A has no effect at the same test concentration.

The use of avenanthramide L in accordance with the present invention exhibits a superior radical-scavenging activity and activity of upregulating the expression and/or gene expression of BLVRB and thus a significant antioxidative capacity and is therefore considered to be useful as an antioxidant. In addition, the antioxidative capacity may be an important mechanism for protecting human skin, hair and nails from environmental, metabolic or pathophysiological stress.

The present compounds, i.e. avenanthramide L, or an oat extract comprising avenanthramide L exhibit established beneficial effects and distinct activity as neurokinin-1 receptor NK1R antagonists, activity for inducing the expression and/or gene expression of small heat shock proteins or for inducing the expression and/or gene expression of CD44 or activity as an antioxidant agent. Due to these promising properties, they have proven useful in both cosmetic and medical applications.

One aspect of the present invention is therefore the use of avenanthramide L or an oat extract comprising avenanthramide L as a cosmetic for skin care, scalp care, hair care, nail care or for use in the prevention and/or treatment of skin condition, intolerant and sensitive skin, skin irritation, skin reddening, wheals, pruritis (itching), skin aging, wrinkle formation, loss of skin volume, loss of skin elasticity, pigment spots, pigment abnormalities, dry skin, i.e. for moisturising the skin.

Another aspect of the present invention relates to avenanthramide L or an oat extract comprising avenanthramide L for use as a medicament.

Due to the aforementioned promising properties, avenanthramide L or an oat extract comprising avenanthramide L is beneficially useful in the prevention and/or treatment of dermatological or keratological diseases, in particular dermatological or keratological diseases having an barrier related, inflammatory, immunoallergic, atherogenic, xerotic or hyperproliferative component. In particular, avenanthramide L or an oat extract comprising avenanthramide L is beneficially useful in the prevention and/or treatment dermatoses, in particular itch and/or itch-related dermatoses.

Examples of such dermatological disorders include eczema, psoriasis, seborrhoea, dermatitis, erythema, pruritis (itching), otitis, xerosis, inflammation, irritation, fibrosis, Lichen planus, Pityriasis rosea, Pityriasis versicolor, autoimmune bullous diseases, urticarial, angiodermal and allergic skin reactions, and wound healing.

Another aspect of the present invention therefore relates to avenanthramide L or an o at extract comprising avenanthramide L for use in the prevention and/or treatment of dermatological or keratological diseases, in particular dermatological or keratological diseases having a barrier related, inflammatory, immunoallergic, atherogenic, xerotic or hyperproliferative component.

Accordingly, the present invention relates to a method for treating dermatological or keratological diseases, in particular dermatological or keratological diseases having an barrier related, inflammatory, immunoallergic, xerotic or hyperproliferative component in a subject in need thereof, wherein the method comprises administering the subject with a therapeutically effective amount of avenanthramide L or an oat extract comprising avenanthramide L in an amount which is sufficient for inhibiting the neurokinin-1 receptor NK1 and/or inducing the expression of small heat shock proteins or the expression of CD44 and/or for inhibiting ROS formation in the subject.

In a preferred variant of the present invention, avenanthramide L or an oat extract comprising avenanthramide L is beneficially useful in the prevention and/or treatment of pruritis (itching).

Chronic pruritis is a common symptom associated with various dermatological conditions and systemic diseases, with no known underlying condition in some cases. Chronic pruritis is classified by clinical presentation (for example, association with diseased/inflamed or normal/non-inflamed skin and/or presence of secondary scratch lesions) and underlying causes (of for example dermatological, systemic, neurological, psychosomatic, mixed or undetermined origin). It is well documented by studies that SP and the neurokinin-1 receptor NK1R play an important role in itch signalling. This is supported by studies demonstrating that: (i) the neurokinin-1 receptor NK1R is broadly expressed in multiple cell types of the skin, such as keratinocytes and mast cells, and the CNS; (ii) in many pruritic dermatological conditions, there is overexpression of the neurokinin-1 receptor NK1R in the epidermis and increased numbers of SP-expressing nerve fibres and inflammatory cells are found in the skin; and (iii) blocking the neurokinin-1 receptor NK1R using neurokinin-1 receptor NK1R antagonists interrupts the transmission of the itch signal, thus reducing itching.

The use of avenanthramide L or an oat extract comprising avenanthramide L for these respective purposes corresponds to a method for imparting the respective therapeutic activity of the substance by adding a therapeutically effective amount of the substance or preparation.

Within the context of the present invention, an effective amount of a composition is the amount of each active component that is sufficient to show a benefit, such as a reduction in a symptom associated with the disorder, disease or condition to be treated. When applied to a combination or a preparation, as in the present case, the term refers to the amount of the combined active ingredients resulting in the benefit.

Another aspect of the present invention relates to the use of avenanthramide L or an oat extract comprising avenanthramide L for preparing foods, food supplements, cosmetic, pharmaceuticals and veterinary preparations useful in the skin care or prevention and/or treatment of said skin conditions or said dermatological or keratological disorders.

Avenanthramide L or an oat extract comprising avenanthramide L can be easily incorporated into conventional foods, food supplements, cosmetic, pharmaceutical or veterinary preparations.

Within this context, the cosmetic and/or dermatological or keratological formulations containing avenanthramide L or an oat extract comprising avenanthramide L can be conventional in composition and serve to treat the skin, hair and/or nails within the context of a dermatological or keratological treatment or cosmetic care.

Since dermatological conditions or diseases are often associated with dry skin, scratched skin, skin lesions or even inflammation, the cosmetic and/or pharmaceutical preparations comprising avenanthramide L or an oat extract comprising avenanthramide L particularly advantageously contain an skin-moisturising and/or moisture-retaining substance, a cooling agent, an osmolyte, a keratolytic substance, a nurturing substance, an anti-inflammatory, antibacterial or antimycotic substance and/or a substance having a reddening-alleviating or itch-alleviating action and/or a lenitive substance.

Within this context, it is also possible and in some cases advantageous to use avenanthramide L or an oat extract comprising avenanthramide L in combination with other active substances, for example with other, optionally even synergistically intensifying or supplementary substances, such as anti-inflammatories, antibacterial or antimycotic substances, substances having a reddening-alleviating or itch-alleviating action, lenitive substances, moisturisers and/or cooling agents and/or antioxidants, preservatives, (metal) chelating agents, penetration enhancers, and/or cosmetically or pharmaceutically acceptable excipients, as in detail described and exemplified below.

Itching occurs with particular intensity especially when the skin is dry. The use of skin moisture regulators in cosmetic or pharmaceutical products can significantly alleviate itching. Hence, within the context of use in accordance with the present invention, cosmetic and/or pharmaceutical preparations comprising avenanthramide L or an oat extract comprising avenanthramide L can also particularly advantageously contain one or more moisturiser regulator(s) and/or moisture-retaining substances, wherein any moisturiser regulator can be used which is suitable or customary in cosmetic and/or pharmaceutical applications, such as: sodium lactate, urea and derivatives, alcohols, alkane diols or alkane triols comprising 3 to 12 carbon atoms, preferably C3 to C10-alkane diols and C3 to C10-alkane triols, more preferably consisting of: glycerol, 1,2-propylene glycol, 1,2-butylene glycol, 1,3-butylene glycol, 1,2-pentanediol, 1,2-hexanediol, 1,2-octanediol and 1,2-decanediol, collagen, elastin or hyaluronic acid, diacyl adipates, petrolatum, urocanic acid, lecithin, panthenol, phytantriol, lycopene, (pseudo-)ceramides, glycosphingolipids, cholesterol, phytosterols, chitosan, chondroitin sulfate, lanolin, lanolin esters, amino acids, alpha-hydroxy acids (e.g. citric acid, lactic acid, malic acid) and derivatives thereof, mono-, di- and oligosaccharides, such as, for example, glucose, galactose, fructose, mannose, laevulose and lactose, polysugars, such as β-glucans, in particular 1,3-1,4-β-glucan from oats or yeast, alpha-hydroxy-fatty acids, triterpenic acids, such as betulic acid or ursolic acid and algae extracts.

Depending on the substance, the concentration of the moisture retention regulators used is between 0.1 and 10% (m/m) and preferably between 0.5 and 5% (m/m), based on the total weight of a ready-to-use cosmetic or pharmaceutical end product. These data apply in particular to such diols as are advantageously to be used, such as hexylene glycol, 1,2-pentanediol, 1,2-hexanediol, 1,2-octanediol and 1,2-decanediol, as well as mixtures of 1,2-hexanediol and 1,2-octanediol.

The use of cooling agents in cosmetic and pharmaceutical products can alleviate itching. Hence, within the context of use in accordance with the present invention, cosmetic and/or pharmaceutical preparations comprising avenanthramide L or an oat extract comprising avenanthramide L can also particularly advantageously contain one or more cooling agent(s). Preferred individual cooling agents for use within the framework of the present invention are listed below. Those skilled in the art can add a large number of other cooling agents to this list; the cooling agents listed can also be used in combination with one another: l-menthol, d-menthol, racemic menthol, menthone glycerol acetal (trade name: Frescolat® MGA), menthyl lactate (trade name: Frescolat® ML; menthyl lactate is preferably l-menthyl lactate, especially l-menthyl l-lactate), substituted menthyl-3-carboxamides (e.g. menthyl-3-carboxylic acid N-ethylamide), 2-isopropyl-N-2,3-trimethylbutanamide, substituted cyclohexanecarboxamides, 3-menthoxypropane-1,2-diol, 2-hydroxyethyl menthyl carbonate, 2-hydroxypropyl menthyl carbonate, N-acetylglycine menthyl ester, isopulegol, menthyl ethylamido oxalate (trade name: Frescolat® X-cool), hydroxycarboxylic acid menthyl esters (e.g. menthyl 3-hydroxybutyrate), monomenthyl succinate, 2-mercaptocyclodecanone, menthyl 2-pyrrolidin-5-onecarboxylate, 2,3-dihydroxy-p-menthane, 3,3,5-trimethylcyclohexanone glycerol ketal, 3-menthyl-3,6-di- and trioxaalkanoates, 3-menthyl methoxy-acetate and icilin.

Cooling agents that are preferred on the basis of their particular synergistic effect are l-menthol, d-menthol, racemic menthol, menthone glycerol acetal (trade name: Frescolat® MGA), menthyl lactate (preferably l-menthyl lactate, especially l-menthyl l-lactate (trade name: Frescolat® ML), substituted menthyl-3-carboxamides (e.g. menthyl-3-carboxylic acid N-ethylamide), 2-isopropyl-N-2,3-trimethylbutanamide, substituted cyclohexanecarboxamides, 3-menthoxy-propane-1,2-diol, 2-hydroxyethyl menthyl carbonate, 2-hydroxypropyl menthyl carbonate, menthyl ethylamido oxalate (trade name: Frescolat® X-cool) and isopulegol. Particularly preferred cooling agents are l-menthol, racemic menthol, menthone glycerol acetal (trade name: Frescolat® MGA), menthyl lactate (preferably l-menthyl lactate, especially l-menthyl l-lactate (trade name: Frescolat® ML), 3-menthoxypropane-1,2-diol, 2-hydroxyethyl menthyl carbonate, menthyl ethylamido oxalate (trade name: Frescolat® X-cool) and 2-hydroxy-propyl menthyl carbonate.

Very particularly preferred cooling agents are l-menthol, menthone glycerol acetal (trade name: Frescolat® MGA), menthyl ethylamido oxalate (trade name: Frescolat® X-cool) and menthyl lactate (preferably l-menthyl lactate, especially l-menthyl l-lactate (trade name: Frescolat® ML).

Depending on the substance, the concentration of the cooling agents used is preferably between 0.01 and 20 wt % and particularly between 0.1 and 5 wt %, based on the total weight of a ready-to-use cosmetic or pharmaceutical end product.

Within the context of use in accordance with the present invention, cosmetic and/or pharmaceutical preparations comprising avenanthramide L or an oat extract comprising avenanthramide L can also particularly advantageously contain one or more osmolyte(s). Examples of osmolytes which may be mentioned here include substances from the group comprising sugar alcohols (myoinositol, mannitol, sorbitol), quaternary amines such as taurine, choline, betaine, betaine glycine, ectoin, diglycerol phosphate, phosphorylcholine or glycerophosphorylcholines, amino acids such as glutamine, glycine, alanine, glutamate, aspartate or proline, phosphatidylcholine, phosphatidylinositol, inorganic phosphates, and polymers of said compounds, such as proteins, peptides, polyamino acids and polyols. All osmolytes simultaneously have a skin-moisturising action.

Preferably, keratolytic substances can also be particularly advantageously used in the cosmetic and/or pharmaceutical preparations comprising avenanthramide L or an oat extract comprising avenanthramide L. Keratolytic compounds include the large group of alpha-hydroxy acids. Salicylic acid is for example preferably used.

In the cosmetic and/or pharmaceutical preparations comprising avenanthramide L or an oat extract comprising avenanthramide L, for the topical cosmetic or pharmaceutical treatment of for example dry and/or itchy skin, a high proportion of in particular nurturing substances is also particularly advantageous because of the reduced trans-epidermal water loss due to lipophilic components. In one preferred embodiment, the cosmetic or pharmaceutical preparations contain one or more nurturing animal and/or vegetable fats and oils such as olive oil, sunflower oil, refined soybean oil, palm oil, sesame oil, rapeseed oil, almond oil, borage oil, evening primrose oil, coconut oil, shea butter, jojoba oil, sperm oil, tallow, neatsfoot oil and lard, and optionally other nurturing components such as fatty alcohols having 8 to 30 C atoms. The fatty alcohols used here can be either saturated or unsaturated and either linear or branched. Nurturing substances which can be particularly preferably combined with the mixtures according to the present invention also include in particular ceramides, understood here to mean N-acylsphingosines (fatty acid amides of sphingosine) or synthetic analogues of such lipids (so-called pseudo-ceramides) which markedly improve the water retention capacity of the stratum corneum; phospholipids, such as soy lecithin, egg lecithin and cephalins; and petrolatum, paraffin oils and silicone oils, the latter including inter alia dialkyl- and alkylarylsiloxanes such as dimethylpolysiloxane and methylphenylpolysiloxane and their alkoxylated and quaternised derivatives.

Within the context of use in accordance with the present invention, the cosmetic and/or pharmaceutical preparations comprising avenanthramide L or a preparation comprising avenanthramide L can also contain one or more anti-inflammatory substance(s) and/or substances that alleviate reddening and/or other substances that alleviate itching, which in this context includes all anti-inflammatory active substances and active substances that alleviate reddening and itching and are suitable and/or conventionally used for cosmetic and/or dermatological applications. Steroidal anti-inflammatory substances of the corticosteroid type, such as hydrocortisone, hydrocortisone derivatives such as hydrocortisone 17-butyrate, dexamethasone, dexamethasone phosphate, methylprednisolone or cortisone, are advantageously used as anti-inflammatory compounds or compounds that alleviate reddening and/or itching; other steroidal anti-inflammatories can also be added to this list. It is also possible to use non-steroidal anti-inflammatories; examples which may be mentioned here include oxicams such as piroxicam or tenoxicam; salicylates such as aspirin, Disalcid®, Solprin® or fendosal; acetic acid derivatives such as diclofenac, fenclofenac, indomethacin, sulindac, tolmetin or clindanac; fenamates such as mefenamic, meclofenamic, flufenamic or niflumic; propionic acid derivatives such as ibuprofen, naproxen or benoxaprofen; or pyrazoles such as phenylbutazone, oxyphenylbutazone, febrazone or azapropazone. A possible alternative is to use natural anti-inflammatory substances or substances that alleviate reddening and/or itching. Plant extracts, special high-activity plant extract fractions and high-purity active substances isolated from plant extracts can be used. Particular preference is afforded to extracts, fractions and active substances from camomile, Aloe vera, Commiphora species, Rubia species, willow, willow-herb, ginger, Glycyrrhiza species, Rubus species, oats, calendula, arnica, St John's wort, honeysuckle, rosemary, Passiflora incarnata, witch hazel, ginger or Echinacea, and pure substances such as, inter alia, (alpha-)bisabolol, apigenin, apigenin-7-glucoside, gingerols such as [6]-gingerol, paradols such as [6]-paradol, boswellic acid, phytosterols, glycyrrhizin, glabridin and licochalcone A. The said formulations can also contain mixtures of two or more anti-inflammatory active compounds.

Depending on the substance, the concentration of the anti-inflammatory compounds which can be used ranges from 0.005 to 2% (m/m) and preferably from 0.05 to 0.5% (m/m), based on the total weight of a ready-to-use cosmetic or pharmaceutical end product. These data apply in particular to bisabolol or synergistic mixtures of bisabolol with ginger extract or with [6]-paradol.

Other antibacterial or antimycotic active substances can also particularly advantageously be used in the cosmetic and/or pharmaceutical preparations containing avenanthramide L or an oat extract comprising avenanthramide L, wherein any antibacterial or antimycotic active substances can be used which are suitable or customary in cosmetic and/or pharmaceutical applications. In addition to the large group of conventional antibiotics, other products which are advantageous here include such as in particular triclosan, climbazole, octoxyglycerin, Octopirox® (1-hydroxy-4-methyl-6-(2,4,4-trimethylpentyl)-2(1H)-pyridone 2-aminoethanol salt), chitosan, farnesol, glycerol monolaurate or combinations of said substances, which are used inter alia against underarm odour, foot odour or dandruff.

Within the context of use in accordance with the present invention, the cosmetic and/or pharmaceutical preparations comprising avenanthramide L or an oat extract comprising avenanthramide L can also contain one or more lenitive substances, wherein any lenitive substances can be used which are suitable or customary in cosmetic and/or pharmaceutical applications such as alpha-bisabolol, azulene, guaiazulene, 18-beta-glycyrrhetinic acid, allantoin, Aloe vera juice or gel, extracts of Hamamelis virginiana (witch hazel), Echinacea species, Centella asiatica, chamomile, Arnica monatana, Glycyrrhiza species, algae, seaweed and Calendula officinalis, and vegetable oils such as sweet almond oil, baobab oil, olive oil and panthenol.

Within the context of use in accordance with the present invention, the cosmetic and/or pharmaceutical preparations comprising avenanthramide L or a preparation comprising avenanthramide L can also contain one or more cosmetically or pharmaceutically acceptable excipients such as those conventionally used in such preparations, for example antioxidants, preservatives, (metal) chelating agents, penetration enhancers, surface-active substances, emulsifiers, perfume oils, anti-foaming agents, colorants, pigments having a colouring action, thickeners, surface-active substances, emulsifiers, plasticisers, other moisturising and/or moisture-retaining substances, fats, oils, waxes or other conventional components of a cosmetic formulation, such as alcohols, polyols, polymers, foam stabilisers, electrolytes, organic solvents or silicone derivatives. Any conceivable antioxidants, preservatives, (metal) chelating agents, penetration enhancers, surface-active substances, emulsifiers, perfume oils, anti-foaming agents, colorants, pigments having a colouring action, thickeners, surface-active substances, emulsifiers, plasticisers, other moisturising and/or moisture-retaining substances, fats, oils, waxes or other conventional components of a cosmetic formulation, such as alcohols, polyols, polymers, foam stabilisers, electrolytes, organic solvents or silicone derivatives that are suitable or conventionally used for cosmetic and/or pharmaceutical applications can be used here in accordance with the invention.

With regard to other cosmetic and pharmaceutical excipients, bases and auxiliaries which can particularly preferably be combined with avenanthramide L or an oat extract comprising avenanthramide L, reference may be made to the detailed descriptions in WO 03/069994, WO 2004/047833 or WO 2007/062957.

Within the context of use in accordance with the present invention, cosmetic and/or pharmaceutical preparations comprising avenanthramide L or an oat extract comprising avenanthramide L can also particularly advantageously contain one or more antioxidant(s), wherein any antioxidants can be used which are suitable or conventionally used for cosmetic and/or pharmaceutical applications. Advantageously, the antioxidants are selected from the group consisting of amino acids (for example glycine, histidine, tyrosine, tryptophan) and their derivatives, imidazoles (for example urocanic acid) and their derivatives, peptides such as D,L-carnosine, D-carnosine, L-carnosine and their derivatives (for example anserine), carotenoids, carotenes (for example α-carotene, β-carotene, lycopene) and their derivatives, lipoic acid and its derivatives (for example dihydrolipoic acid), aurothioglucose, propylthiouracil and other thiols (for example thioredoxin, glutathione, cysteine, cystine, cystamine and their glycosyl, N-acetyl, methyl, ethyl, propyl, amyl, butyl and lauryl, palmitoyl, oleyl, γ-linoleyl, cholesteryl and glyceryl esters) and their salts, dilauryl thiodipropionate, distearyl thiodipropionate, thiodipropionic acid and their derivatives (esters, ethers, peptides, lipids, nucleotides, nucleosides and salts) as well as sulphoximine compounds (for example buthionine sulphoximines, homocysteine sulphoximines, buthionine sulphones, penta-, hexa-, hepta-thionine sulphoximine) in very low tolerated doses, and also (metal) chelating agents, for example α-hydroxy fatty acids, palmitic acid, phytic acid, lactoferrin, α-hydroxy acids (for example citric acid, lactic acid, malic acid), humic acid, bile acid, bile extracts, bilirubin, biliverdin, EDTA, EGTA and their derivatives, unsaturated fatty acids and their derivatives (for example γ-linolenic acid, linoleic acid, oleic acid), folic acid and its derivatives, ubiquinone and ubiquinol and their derivatives, Vitamin C and its derivatives (for example ascorbyl palmitate, magnesium ascorbyl phosphate, ascorbyl acetate), tocopherols and their derivatives (for example Vitamin E acetate), Vitamin A and its derivatives (for example Vitamin A palmitate) and also coniferyl benzoate of benzoin resin, rutinic acid and its derivatives, ferrulic acid and its derivatives, butylhydroxytoluene, butylhydroxyanisole, nordihydroguaiacic acid, nordihydroguaiaretic acid, trihydroxybutyrophenone, uric acid and its derivatives, mannose and its derivatives, zinc and its derivatives (for example ZnO, ZnSO₄), gingerols e.g. [6]-gingerol, paradols e.g. [6]-paradol, selenium and its derivatives (such as selenium methionine), stilbenes and their derivatives (such as stilbene oxide, trans-stilbene oxide), as well as the derivatives (such as salts, esters, ethers, sugars, nucleotides, nucleosides, peptides and lipids) of said active compounds such as are suitable in accordance with the invention.

Within the context of use in accordance with the present invention, cosmetic and/or pharmaceutical preparations comprising avenanthramide L or an oat extract comprising avenanthramide L can also particularly advantageously contain one or more substance(s) for preservative purposes, wherein any preservatives may be used which are suitable or customary in cosmetic and/or pharmaceutical applications and which are advantageously selected from the group consisting of preservatives such as inter alia benzoic acid, its esters and salts; propionic acid and its salts; salicylic acid and its salts; 2,4-hexanoic acid (sorbic acid) and its salts; formaldehyde and paraformaldehyde; 2-hydroxybiphenyl ether and its salts; 2-zincsulphidopyridine N-oxide; inorganic sulphites and bisulphites; sodium iodate; chlorobutanol; 4-hydroxybenzoic acid and its salts and esters; dehydroacetic acid; formic acid; 1,6-bis(4-amidino-2-bromophenoxy)-n-hexane and its salts; the sodium salt of ethylmercury-(II)-thiosalicylic acid; phenylmercury and its salts; 10-undecylenic acid and its salts; 5-amino-1,3-bis(2-ethylhexyl)-5-methylhexahydropyrimidine; 5-bromo-5-nitro-1,3-dioxane; 2-bromo-2-nitro-1,3-propanediol; 2,4-dichlorobenzyl alcohol; N-(4-chlorophenyl)-N′-(3,4-dichlorophenyl)urea; 4-chloro-m-cresol; 2,4,4′-trichloro-2′-hydroxy-diphenyl ether; 4-chloro-3,5-dimethylphenol; 1,1′-methylene-bis(3-(1-hydroxymethyl-2,4-dioximidazolidin-5-yl)urea); poly(hexamethylene biguanide) hydrochloride; 2-phenoxyethanol; hexamethylenetetramine; 1-(3-chloroallyl)-3,5,7-triaza-1-azoniaadamantane chloride; 1-(4-chloro-phenoxy)-1(1H-imidazol-1-yl)-3,3-dimethyl-2-butanone; 1,3-bis(hydroxymethyl)-5,5-dimethyl-2,4-imidazolidinedione; benzyl alcohol; Octopirox®; 1,2-dibromo-2,4-dicyanobutane; 2,2′-methylene-bis(6-bromo-4-chloro-phenol); bromochlorophene; mixture of 5-chloro-2-methyl-3(2H)-isothiazolinone and 2-methyl-3(2H)isothiazolinone with magnesium chloride and magnesium nitrate; 2-benzyl-4-chlorophenol; 2-chloroacetamide; chlorhexidine; chlorhexidine acetate; chlorhexidine gluconate; chlorhexidine hydrochloride; 1-phenoxy-propan-2-ol; N-alkyl(C12-C22)trimethylammonium bromide and chloride; 4,4-dimethyl-1,3-oxazolidine; N-hydroxymethyl-N-(1,3-di(hydroxymethyl)-2,5-dioxoimidazolidin-4-yl)-N′-hydroxymethylurea; 1,6-bis(4-amidinophenoxy)-n-hexane and its salts; glutaraldehyde 5-ethyl-1-aza-3,7-dioxabicyclo(3.3.0)octane; 3-(4-chlorophenoxy)-1,2-propanediol; hyamine; alkyl(C8-C18)dimethylbenzylammonium chloride; alkyl(C8-C18)dimethylbenzylammonium bromide; alkyl(C8-C18)dimethylbenzylammonium saccharinate; benzylhemiformal; 3-iodo-2-propynyl butylcarbamate; o-cymen-5-ol, or sodium ((hydroxymethyl)amino)acetate.

Within the context of use in accordance with the present invention, cosmetic and/or pharmaceutical preparations comprising avenanthramide L or an oat extract comprising avenanthramide L can also particularly advantageously contain one or more (metal) chelating agent(s), wherein any metal chelating agents can be used which are suitable or customary in cosmetic and/or pharmaceutical applications. Preferred (metal) chelating agents include α-hydroxy fatty acids, phytic acid, lactoferrin, α-hydroxy acids, such as inter alia citric acid, lactic acid and malic acid, as well as humic acids, bile acids, bile extracts, bilirubin, biliverdin or EDTA, EGTA and their derivatives.

Within the context of use in accordance with the present invention, cosmetic and/or pharmaceutical preparations comprising avenanthramide L or an oat extract comprising avenanthramide L can also particularly advantageously contain one or more penetration enhancers, wherein any penetration enhancer can be used which is suitable or customary in cosmetic and/or pharmaceutical applications. Penetration enhancers may enhance the penetration of the active substance(s) through the skin. Preferred penetrations enhancers include sulphoxides (such as dimethyl sulphoxide, DMSO), fatty acids (such as caprylic, capric, lauric, myristic, palmitic, stearic, oleic and linoleic acid), fatty esters (such as ethyl oleate, ethyl laurate) and fatty alcohols (such as capryl, decyl, lauryl, myristyl, cetyl, stearyl, oleyl, linoleyl alcohol), azones (such as laurocapram), pyrrolidones (for example 2-pyrrolidone, 2P), alcohols and alkanols (such as ethanol, propanol, butanol or decanol), glycerols, terpenes (such as 1,8-cineole, limonene, menthone, nerolidol, linalool, and menthol), surfactants (such as SDS and SLS), urea, dimethyl isosorbide. Preferred penetration enhancers used in accordance with the present invention are 1,2-propanediol (propylene glycol), 1,2-butanediol, 1,2-pentanediol (Hydrolite-5), 1,2-hexanediol (Hydrolite 6), 1,2-heptanediol, 1,2-octanediol, 1,2-nonanediol, 1,2-decanediol or 1,2-dodecane diol; 1-3-butanediol (butylene glycol), 1,4-butanediol, 1,1′oxydi-2-propanol (dipropylene glycol) and its isomers; 1,3-propanediol; polyols, alcohol; dimethyl isosorbide (INCI); triethyl citrate; butylene carbonate; glycerine carbonate; dipropylene glycol or any mixtures of these.

Within the context of use in accordance with the present invention, cosmetic and/or pharmaceutical preparations comprising avenanthramide L or an oat extract comprising avenanthramide L can also particularly advantageously contain one or more anionic, cationic, non-ionic and/or amphoteric surfactant(s), in particular if crystalline or microcrystalline solids, for example inorganic micropigments, are to be incorporated into the preparations. Surfactants are amphiphilic substances capable of solubilising organic, non-polar substances in water. The hydrophilic parts of a surfactant molecule are usually polar functional groups, such as —COO⁻, —OSO₃⁻ or —SO₃⁻, while the hydrophobic parts are normally non-polar hydrocarbon radicals. Surfactants are generally classified according to the type and charge of the hydrophilic part of the molecule. They can be divided into four groups: anionic surfactants, cationic surfactants; amphoteric surfactants; and non-ionic surfactants.

Anionic surfactants normally contain carboxylate, sulphate or sulphonate groups as functional groups. In aqueous solution, they form negatively charged organic ions in an acidic or neutral medium. Cationic surfactants are characterised virtually exclusively by the presence of a quaternary ammonium group. In aqueous solution they form positively charged organic ions in an acidic or neutral medium. Amphoteric surfactants contain both anionic and cationic groups and accordingly behave like anionic or cationic surfactants in aqueous solution, depending on the pH value. They have a positive charge in a strongly acidic medium and a negative charge in an alkaline medium. In the neutral pH range, by contrast, they are zwitterionic. Polyether chains are typical of non-ionic surfactants. Non-ionic surfactants do not form ions in an aqueous medium.

Anionic surfactants that can advantageously be used include: acyl amino acids (and their salts), such as: acyl glutamates, for example sodium acyl glutamate, di-TEA-palmitoyl aspartate and sodium caprylic/capric glutamate; acyl peptides, for example palmitoyl-hydrolysed lactoprotein, sodium cocoyl-hydrolysed soy protein and sodium/potassium cocoyl-hydrolysed collagen; sarcosinates, for example myristoyl sarcosinate, TEA-lauroyl sarcosinate, sodium lauroyl sarcosinate and sodium cocoyl sarcosinate; taurates, for example sodium lauroyl taurate and sodium methyl cocoyl taurate; acyl lactylates, for example lauroyl lactylate and caproyl lactylate; alaninates; carboxylic acids and derivatives, such as for example lauric acid, aluminium stearate, magnesium alkanolate and zinc undecylenate; ester carboxylic acids, for example calcium stearoyl lactylate, laureth-6 citrate and sodium PEG-4 lauramide carboxylate; ether carboxylic acids, for example sodium laureth-13 carboxylate and sodium PEG-6 cocamide carboxylate; phosphoric acid esters and salts, such as for example DEA-oleth-10 phosphate and dilaureth-4 phosphate; sulphonic acids and salts, such as acyl isethionates, for example sodium/ammonium cocoyl isethionate; alkyl aryl sulphonates; alkyl sulphonates, for example sodium cocomonoglyceride sulphonate, sodium C12-14 olefin sulphonate, sodium lauryl sulphoacetate and magnesium PEG-3 cocamide sulphate; sulphosuccinates, for example dioctyl sodium sulphosuccinate, disodium laureth sulphosuccinate, disodium lauryl sulphosuccinate and disodium undecylenamido MEA-sulphosuccinate; and sulphuric acid esters, such as alkyl ether sulphate, for example sodium, ammonium, magnesium, MIPA, TIPA laureth sulphate, sodium myreth sulphate and sodium C12-13 pareth sulphate, and alkyl sulphates, for example sodium, ammonium and TEA lauryl sulphate.

Cationic surfactants that can advantageously be used include alkyl amines, alkyl imidazoles, ethoxylated amines and quaternary surfactants: RNH₂CH₂CH₂COO⁻ (at pH 7); RNHCH₂CH₂COO⁻B⁺ (at pH 12), where B⁺ is arbitrary cation, such as Na⁺;

esterquats.

Quaternary surfactants contain at least one N atom that is covalently bonded to four alkyl or aryl groups. This leads to a positive charge, irrespective of the pH value. Alkyl betaine, alkyl amidopropyl betaine and alkyl amidopropyl hydroxysulphaine are advantageous. The cationic surfactants used can also preferably be chosen from the group of quaternary ammonium compounds, in particular benzyl trialkyl ammonium chlorides or bromides, such as for example benzyl dimethylstearyl ammonium chloride, as well as alkyl trialkyl ammonium salts, for example cetyl trimethyl ammonium chloride or bromide, alkyl dimethyl hydroxyethyl ammonium chlorides or bromides, dialkyl dimethyl ammonium chlorides or bromides, alkyl amide ethyl trimethyl ammonium ether sulphates, alkyl pyridinium salts, for example lauryl or cetyl pyridinium chloride, imidazoline derivatives and compounds of a cationic nature, such as amine oxides, for example alkyl dimethyl amine oxides or alkyl aminoethyl dimethyl amine oxides. Cetyl trimethyl ammonium salts can particularly advantageously be used.

Amphoteric surfactants that can advantageously be used include: acyl/dialkyl ethylene diamine, for example sodium acyl amphoacetate, disodium acyl amphodipropionate, disodium alkyl amphodiacetate, sodium acyl amphohydroxypropyl sulphonate, disodium acyl amphodiacetate and sodium acyl amphopropionate; N-alkyl amino acids, for example aminopropyl alkyl glutamide, alkyl aminopropionic acid, sodium alkyl imidodipropionate and lauroamphocarboxyglycinate.

Non-ionic surfactants that can advantageously be used include: alcohols; alkanolamides, such as cocamides MEA/DEA/MIPA, amine oxides, such as cocoamidopropylamine oxide; esters formed by esterification of carboxylic acids with ethylene oxide, glycerol, sorbitan or other alcohols; ethers, for example ethoxylated/propoxylated alcohols, ethoxylated/propoxylated esters, ethoxylated/propoxylated glycerol esters, ethoxylated/propoxylated cholesterols, ethoxylated/propoxylated triglyceride esters, ethoxylated/propoxylated lanolin, ethoxylated/propoxylated polysiloxanes, propoxylated polyoxyethylene (POE) ethers and alkyl polyglycosides, such as lauryl glucoside, decyl glycoside and cocoglycoside; sucrose esters and ethers; polyglycerol esters, diglycerol esters, monoglycerol esters; methyl glucose esters, esters of hydroxy acids.

The use of a combination of anionic and/or amphoteric surfactants with one or more non-ionic surfactants is also advantageous.

The surface-active substance can be present at a concentration of between 1 and 98% (m/m) in the preparations containing avenanthramide L or an oat extract comprising avenanthramide L, based on the total weight of the preparations.

Within the context of use in accordance with the present invention, cosmetic and/or pharmaceutical preparations comprising avenanthramide L or an oat extract comprising avenanthramide L can also particularly advantageously contain one or more emulsifiers commonly used in the art for preparing cosmetic or pharmaceutical preparations. Oil-in-water (O/W) emulsifiers can for example be advantageously selected from the group comprising polyethoxylated or polypropoxylated or polyethoxylated and polypropoxylated products, such as fatty alcohol ethoxylates, ethoxylated wool wax alcohols, polyethylene glycol ethers of the general formula R—O—(—CH₂—CH₂—O—)ₙ—R′, fatty acid ethoxylates of the general formula R—COO—(—CH₂—CH₂—O—)ₙ—H, etherified fatty acid ethoxylates of the general formula R—COO—(—CH₂—CH₂—O—)ₙ—R′, esterified fatty acid ethoxylates of the general formula R—COO—(—CH₂—CH₂—O—)ₙ—C(O)—R′, polyethylene glycol glycerol fatty acid esters, ethoxylated sorbitan esters, cholesterol ethoxylates, ethoxylated triglycerides, alkyl ether carboxylic acids of the general formula R—COO—(—CH₂—CH₂—O—)ₙ—OOH, where n is a number from 5 to 30, polyoxyethylene sorbitol fatty acid esters, alkyl ether sulphates of the general formula R—O—(—CH₂—CH₂—O—)ₙ—SO₃—H, fatty alcohol propoxylates of the general formula R—O—(—CH₂—CH(CH₃)—O—)ₙ—H, polypropylene glycol ethers of the general formula R—O—(—CH₂—CH(CH₃)—O—)ₙ—R′, propoxylated wool wax alcohols, etherified fatty acid propoxylates R—COO—(—CH₂—CH(CH₃)—O—)ₙ—R′, esterified fatty acid propoxylates of the general formula R—COO—(—CH₂—CH(CH₃)—O—)ₙ—C(O)—R′, fatty acid propoxylates of the general formula R—COO—(—CH₂—CH(CH₃)—O—)ₙ—H, polypropylene glycol glycerol fatty acid esters, propoxylated sorbitan esters, cholesterol propoxylates, propoxylated triglycerides, alkyl ether carboxylic acids of the general formula R—O—(—CH₂—CH(CH₃)—O—)ₙ—CH₂—COOH, alkyl ether sulphates (and the acids on which these sulphates are based) of the general formula R—O—(—CH₂—CH(CH₃)—O—)ₙ—SO₃—H, fatty alcohol ethoxylates/propoxylates of the general formula R—O—Xₙ—Yₘ—H, polypropylene glycol ethers of the general formula R—O—Xₙ—Yₙ—R′, etherified fatty acid propoxylates of the general formula R—COO—Xₙ—Yₙ—R′, and fatty acid ethoxylates/propoxylates of the general formula R—COO—Xₙ—Yₘ—H.

In accordance with the invention, the polyethoxylated or polypropoxylated or polyethoxylated and polypropoxylated O/W emulsifiers used are particularly advantageously selected from the group comprising substances having HLB values of 11 to 18, more particularly advantageously 14.5 to 15.5, if the O/W emulsifiers contain saturated radicals R and R′. If the O/W emulsifiers contain unsaturated radicals R and/or R′, or if isoalkyl derivatives are present, then the preferred HLB value of such emulsifiers can also be lower or higher. The fatty alcohol ethoxylates are advantageously selected from the group comprising ethoxylated stearyl alcohols, cetyl alcohols and cetylstearyl alcohols (cetearyl alcohols).

The following emulsifiers are particularly preferred: polyethylene glycol (13) stearyl ether (steareth-13), polyethylene glycol (14) stearyl ether (steareth-14), polyethylene glycol (15) stearyl ether (steareth-15), polyethylene glycol (16) stearyl ether (steareth-16), polyethylene glycol (17) stearyl ether (steareth-17), polyethylene glycol (18) stearyl ether (steareth-18), polyethylene glycol (19) stearyl ether (steareth-19), polyethylene glycol (20) stearyl ether (steareth-20), polyethylene glycol (12) isostearyl ether (isosteareth-12), polyethylene glycol (13) isostearyl ether (isosteareth-13), polyethylene glycol (14) isostearyl ether (isosteareth-14), polyethylene glycol (15) isostearyl ether (isosteareth-15), polyethylene glycol (16) isostearyl ether (isosteareth-16), polyethylene glycol (17) isostearyl ether (isosteareth-17), polyethylene glycol (18) isostearyl ether (isosteareth-18), polyethylene glycol (19) isostearyl ether (isosteareth-19), polyethylene glycol (20) isostearyl ether (isosteareth-20), polyethylene glycol (13) cetyl ether (ceteth-13), polyethylene glycol (14) cetyl ether (ceteth-14), polyethylene glycol (15) cetyl ether (ceteth-15), polyethylene glycol (16) cetyl ether (ceteth-16), polyethylene glycol (17) cetyl ether (ceteth-17), polyethylene glycol (18) cetyl ether (ceteth-18), polyethylene glycol (19) cetyl ether (ceteth-19), polyethylene glycol (20) cetyl ether (ceteth-20), polyethylene glycol (13) isocetyl ether (isoceteth-13), polyethylene glycol (14) isocetyl ether (isoceteth-14), polyethylene glycol (15) isocetyl ether (isoceteth-15), polyethylene glycol (16) isocetyl ether (isoceteth-16), polyethylene glycol (17) isocetyl ether (isoceteth-17), polyethylene glycol (18) isocetyl ether (isoceteth-18), polyethylene glycol (19) isocetyl ether (isoceteth-19), polyethylene glycol (20) isocetyl ether (isoceteth-20), polyethylene glycol (12) oleyl ether (oleth-12), polyethylene glycol (13) oleyl ether (oleth-13), polyethylene glycol (14) oleyl ether (oleth-14), polyethylene glycol (15) oleyl ether (oleth-15), polyethylene glycol (12) lauryl ether (laureth-12), polyethylene glycol (12) isolauryl ether (isolaureth-12), polyethylene glycol (13) cetylstearyl ether (ceteareth-13), polyethylene glycol (14) cetylstearyl ether (ceteareth-14), polyethylene glycol (15) cetylstearyl ether (ceteareth-15), polyethylene glycol (16) cetylstearyl ether (ceteareth-16), polyethylene glycol (17) cetylstearyl ether (ceteareth-17), polyethylene glycol (18) cetylstearyl ether (ceteareth-18), polyethylene glycol (19) cetylstearyl ether (ceteareth-19) and polyethylene glycol (20) cetylstearyl ether (ceteareth-20).

The fatty acid ethoxylates are also advantageously selected from the following group: polyethylene glycol (20) stearate, polyethylene glycol (21) stearate, polyethylene glycol (22) stearate, polyethylene glycol (23) stearate, polyethylene glycol (24) stearate, polyethylene glycol (25) stearate, polyethylene glycol (12) isostearate, polyethylene glycol (13) isostearate, polyethylene glycol (14) isostearate, polyethylene glycol (15) isostearate, polyethylene glycol (16) isostearate, polyethylene glycol (17) isostearate, polyethylene glycol (18) isostearate, polyethylene glycol (19) isostearate, polyethylene glycol (20) isostearate, polyethylene glycol (21) isostearate, polyethylene glycol (22) isostearate, polyethylene glycol (23) isostearate, polyethylene glycol (24) isostearate, polyethylene glycol (25) isostearate, polyethylene glycol (12) oleate, polyethylene glycol (13) oleate, polyethylene glycol (14) oleate, polyethylene glycol (15) oleate, polyethylene glycol (16) oleate, polyethylene glycol (17) oleate, polyethylene glycol (18) oleate, polyethylene glycol (19) oleate and polyethylene glycol (20) oleate.

Sodium laureth-11 carboxylate can advantageously be used as an ethoxylated alkyl ether carboxylic acid or its salt. Sodium laureth-14 sulphate can advantageously be used as an alkyl ether sulphate. Polyethylene glycol (30) cholesteryl ether can advantageously be used as an ethoxylated cholesterol derivative. Polyethylene glycol (25) soya sterol has also proven useful.

Polyethylene glycol (60) evening primrose glycerides can advantageously be used as ethoxylated triglycerides.

The polyethylene glycol glycerol fatty acid esters are also advantageously selected from the group comprising polyethylene glycol (20) glyceryl laurate, polyethylene glycol (21) glyceryl laurate, polyethylene glycol (22) glyceryl laurate, polyethylene glycol (23) glyceryl laurate, polyethylene glycol (6) glyceryl caprylate/caprate, polyethylene glycol (20) glyceryl oleate, polyethylene glycol (20) glyceryl isostearate and polyethylene glycol (18) glyceryl oleate/cocoate.

The sorbitan esters are likewise favourably selected from the group comprising polyethylene glycol (20) sorbitan monolaurate, polyethylene glycol (20) sorbitan monostearate, polyethylene glycol (20) sorbitan monoisostearate, polyethylene glycol (20) sorbitan monopalmitate and polyethylene glycol (20) sorbitan monooleate.

The following can be used as advantageous W/O emulsifiers: fatty alcohols having 8 to 30 carbon atoms; monoglycerol esters of saturated and/or unsaturated, branched and/or unbranched alkane carboxylic acids having a chain length of 8 to 24, in particular 12 to 18 C atoms; diglycerol esters of saturated and/or unsaturated, branched and/or unbranched alkane carboxylic acids having a chain length of 8 to 24, in particular 12 to 18 C atoms; monoglycerol ethers of saturated and/or unsaturated, branched and/or unbranched alcohols having a chain length of 8 to 24, in particular 12 to 18 C atoms; diglycerol ethers of saturated and/or unsaturated, branched and/or unbranched alcohols having a chain length of 8 to 24, in particular 12 to 18 C atoms; propylene glycol esters of saturated and/or unsaturated, branched and/or unbranched alkane carboxylic acids having a chain length of 8 to 24, in particular 12 to 18 C atoms; and sorbitan esters of saturated and/or unsaturated, branched and/or unbranched alkane carboxylic acids having a chain length of 8 to 24, in particular 12 to 18 C atoms.

Particularly advantageous W/O emulsifiers include: glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol (2) stearyl ether (steareth-2), glyceryl monolaurate, glyceryl monocaprate and glyceryl monocaprylate.

Within the context of use in accordance with the present invention, avenanthramide L or an oat extract comprising avenanthramide L can also be used as a component of perfume compositions for hair and scalp care products and, in particular because of their specific efficacy, can impart an additional itch-alleviating or antiallergic property to for example a perfumed finished product. Particularly preferred perfume compositions comprise (a) a sensorially effective amount of a perfume, (b) an itch-regulating, antiallergic and/or hyposensitising amount of a synergistically effective mixture of anthranilic acid amides and antidandruff agents, and (c) optionally, one or more excipients and/or additives. It has proven particularly advantageous that avenanthramide L or an oat extract comprising avenanthramide L have only a weak inherent odour or are even completely odourless, since this property lends them to use in a perfume composition in particular.

Avenanthramide L or an oat extract comprising avenanthramide L can be incorporated without difficulty into conventional cosmetic or dermatological or keratological formulations such as inter alia pump sprays, aerosol sprays, creams, shampoos, ointments, tinctures, lotions, nail care products (such as nail varnishes, nail varnish removers, nail balsams) and the like. Within this context, it is also possible and in some cases advantageous to combine the synergistically effective combinations of anthranilic acid amides and antidandruff agents with other active compounds. Within this context, the cosmetic and/or dermatological or keratological formulations containing avenanthramide L or an oat extract comprising avenanthramide L can otherwise be conventional in composition and can be used for treating the skin, hair and/or nails within the context of cosmetic care or dermatological or keratological treatment.

If the cosmetic or pharmaceutical preparation is a solution or lotion, then solvents which can be used include: water or aqueous solutions; fatty oils, fats, waxes and other natural and synthetic fatty bodies, preferably esters of fatty acids with alcohols having a low C number, such as isopropanol, propylene glycol or glycerol, or esters of fatty alcohols with alkanoic acids having a low C number or with fatty acids; alcohols, diols or polyols having a low C number, and their ethers, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products. Mixtures of the abovementioned solvents are in particular used. In the case of alcoholic solvents, water can be an additional constituent.

The cosmetic or pharmaceutical preparations can also be formulated in a form suitable for topical application, for example as lotions, aqueous or aqueous-alcoholic gels, vesicle dispersions or as simple or complex emulsions (O/W, W/O, O/W/O or W/O/W), liquids, semi-liquids or solids, such as milks, creams, gels, cream-gels, pastes or sticks, and can optionally be packaged as an aerosol and take the form of mousses or sprays. Such formulations are prepared according to usual methods.

For preparing emulsions, the oil phase can advantageously be chosen from the following group of substances: mineral oils, mineral waxes; fatty oils, fats, waxes and other natural and synthetic fatty bodies, preferably esters of fatty acids with alcohols having a low C number, for example with isopropanol, propylene glycol or glycerol, or esters of fatty alcohols with alkanoic acids having a low C number or with fatty acids; alkyl benzoates; silicone oils such as dimethyl polysiloxanes, diethyl polysiloxanes, diphenyl polysiloxanes and mixed forms thereof.

Advantageously, esters of saturated and/or unsaturated, branched and/or straight-chain alkane carboxylic acids having a chain length of 3 to 30 C atoms and saturated and/or unsaturated, branched and/or straight-chain alcohols having a chain length of 3 to 30 C atoms, from the group of esters of aromatic carboxylic acids and saturated and/or unsaturated, branched and/or straight-chain alcohols having a chain length of 3 to 30 C atoms can be used. Preferred ester oils include isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and natural mixtures of such esters, for example jojoba oil.

In addition, the oily phase can advantageously be selected from the group comprising branched and unbranched hydrocarbons and waxes, silicone oils, dialkyl ethers, the group comprising saturated or unsaturated, branched or unbranched alcohols, and also fatty acid triglycerides, specifically the triglycerol esters of saturated and/or unsaturated, branched and/or unbranched alkane carboxylic acids having a chain length of 8 to 24 and in particular 12 to 18 C atoms. The fatty acid triglycerides can advantageously be selected from the group comprising synthetic, semi-synthetic and natural oils, such as olive oil, sunflower oil, soybean oil, peanut oil, rapeseed oil, almond oil, palm oil, coconut oil, palm kernel oil and the like. Arbitrary mixtures of such oil and wax components can also advantageously be used. In some cases, it is also advantageous to use waxes, such as cetyl palmitate, as the sole lipid component of the oily phase; advantageously, the oily phase is selected from the group comprising 2-ethylhexyl isostearate, octyldodecanol, isotridecyl isononanoate, isoeicosane, 2-ethylhexyl cocoate, C12-15 alkyl benzoate, caprylic/capric triglyceride and dicaprylyl ether. Mixtures of C12-15 alkyl benzoate and 2-ethylhexyl isostearate, mixtures of C12-15 alkyl benzoate and isotridecyl isononanoate and mixtures of C12-15 alkyl benzoate, 2-ethylhexyl isostearate and isotridecyl isononanoate are particularly advantageous. The hydrocarbons paraffin oil, squalane and squalene can also advantageously be used. The oily phase can advantageously also contain cyclic or linear silicone oils or consist entirely of such oils, although other oily phase components are preferably used in addition to the silicone oil(s). Cyclomethicone (for example, decamethylcyclopentasiloxane) can advantageously be used as a silicone oil. However, other silicone oils can also advantageously be used, including for example undecamethylcyclotrisiloxane, polydimethylsiloxane and poly(methylphenylsiloxane). Mixtures of cyclomethicone and isotridecyl isononanoate and of cyclomethicone and 2-ethylhexyl isostearate are also particularly advantageous.

The aqueous phase of preparations containing avenanthramide L or an oat extract comprising avenanthramide L and taking the form of an emulsion can advantageously comprise alcohols, diols or polyols having a low C number, as well as their ethers, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and also alcohols having a low C number, such as ethanol, isopropanol, 1,2-propanediol and glycerol, and in particular one or more thickeners, which can advantageously be selected from the group comprising silicon dioxide, aluminium silicates, polysaccharides and their derivatives, such as hyaluronic acid, xanthan gum, hydroxypropyl methyl cellulose, and particularly advantageously from the group comprising polyacrylates, preferably a polyacrylate from the group comprising so-called carbopols, such as type 980, 981, 1382, 2984 and 5984 carbopols, each on their own or in combinations.

A high content of treatment substances is usually advantageous in preparations containing avenanthramide L or an oat extract comprising avenanthramide L for the topical prophylactic or cosmetic treatment of the skin. In accordance with a preferred variant, the compositions contain one or more animal and/or vegetable treatment fats and oils, such as olive oil, sunflower oil, purified soybean oil, palm oil, sesame oil, rapeseed oil, almond oil, borage oil, evening primrose oil, coconut oil, shea butter, jojoba oil, sperm oil, beef tallow, neatsfoot oil and lard, and optionally other treatment constituents such as for example C8-C30 fatty alcohols. The fatty alcohols used here can be saturated or unsaturated and straight-chain or branched, wherein examples include decanol, decenol, octanol, octenol, dodecanol, dodecenol, octadienol, decadienol, dodecadienol, oleyl alcohol, ricinoleyl alcohol, erucic alcohol, stearyl alcohol, isostearyl alcohol, cetyl alcohol, lauryl alcohol, myristyl alcohol, arachidyl alcohol, capryl alcohol, capric alcohol, linoleyl alcohol, linolenyl alcohol and behenyl alcohol, as well their guerbet alcohols; this list may be extended as desired to include other alcohols which structurally are chemically related. The fatty alcohols preferably originate from natural fatty acids and are usually prepared from the corresponding esters of the fatty acids by reduction. Fatty alcohol fractions formed by reduction from naturally occurring fats and fat oils can also be used, such as for example beef tallow, peanut oil, colza oil, cottonseed oil, soybean oil, sunflower oil, palm kernel oil, linseed oil, maize oil, castor oil, rapeseed oil, sesame oil, cocoa butter and cocoa fat.

The treatment substances that can preferably be combined with the composition or oat extract according to the present invention can also include: ceramides, being understood to be N-acylsphingosines (fatty acid amides of sphingosine) or synthetic analogues of such lipids (so-called pseudo-ceramides) which clearly improve the water retention capacity of the stratum corneum; phospholipids, for example soy lecithin, egg lecithin and cephalins; Vaseline, paraffin and silicone oils, the latter including inter alia dialkyl- and alkylaryl-siloxanes such as dimethylpolysiloxane and methylphenylpolysiloxane, as well as their alkoxylated and quaternised derivatives.

Hydrolysed animal and/or vegetable proteins can also advantageously be added to the formulations containing the composition or oat extract according to the present invention. Advantageous examples in this regard include in particular elastin, collagen, keratin, lactoprotein, soy protein, oat protein, pea protein, almond protein and wheat protein fractions or corresponding hydrolysed proteins, as well as their condensation products with fatty acids, and also quaternised hydrolysed proteins, wherein the use of hydrolysed vegetable proteins is preferred.

The cosmetic or pharmaceutical preparations containing avenanthramide L or an oat extract comprising avenanthramide L may also include a cosmetically or pharmaceutically acceptable carrier, such as (without being limited to) one of the following which are commonly used in the art: lactose, dextrose, sucrose, sorbitol, mannitol, starch, acacia rubber, calcium phosphate, alginate, gelatine, calcium silicate, microcrystalline cellulose, polyvinyl pyrrolidone, cellulose, water, syrup, methyl cellulose, methyl hydroxybenzoate, propyl hydroxybenzoate, talc, magnesium stearate, mineral oil and the like. The cosmetic or pharmaceutical preparations may also include lubricants, wetting agents, sweeteners, flavouring agents, emulsifiers, suspensions, preserving agents and the like, in addition to the above components. Suitable pharmaceutically acceptable carriers and formulations are described in detail in Remington's Pharmaceutical Sciences (19^{th} edition, 1995).

In a preferred variant, the foods, food supplements, cosmetic, pharmaceutical or veterinary preparations comprise avenanthramide L or an oat extract comprising avenanthramide L in an amount of 0.0001 to 10 wt %, preferably 0.0005 to 5 wt %, and more preferred 0.001 to 1 wt %, based on the total weight of the preparation or final composition.

In order to be used, the cosmetic or pharmaceutical preparations containing avenanthramide L or a preparation comprising avenanthramide L are applied to the skin, hair and/or nails in an adequate amount and in such manner as is customary with cosmetics or pharmaceutical products.

Because of its significant effect in inhibiting the neurokinin-1 receptor NK1R, avenanthramide L or an oat extract comprising avenanthramide L is suitable as a neurokinin-1 receptor NK1R antagonist.

Thus, in accordance with another aspect, the present invention relates to avenanthramide L or an oat extract comprising avenanthramide L as an neurokinin-1 receptor NK1R antagonist.

Finally, the present invention relates to a method for preparing avenalumic acid and/or avenanthramide L, comprising the steps of:
(a) reacting triethyl phosphite (1) and methyl 4-bromocrotonate (2) to form methyl (2E)-4-(diethylphosphoryl)but-2-enoate (3);
(b) reacting methyl (2E)-4-(diethylphosphoryl)but-2-enoate (3) in an HWE reaction with 4-formylphenyl acetate (4) to form methyl (2E, 4E)-5-(4-hydroxyphenyl)penta-2,4-dienoate (5);
(c) deprotecting avenalumic acid methyl ester (5), using a sodium hydroxide solution, to yield avenalumic acid (Avn Ac); and
(d) reacting avenalumic acid (Avn Ac) with 2-amino-5-hydroxybenzoic acid (6), using coupling reagents and without using any protecting groups, to yield avenanthramide L (Avn L).

For the synthesis of avenalumic acid (Avn Ac), the known procedure from Li Y. et al., Food Chemistry 2014, 158, 41-47 was used with minor modifications, starting from triethyl phosphite (1) and methyl 4-bromocrotonate (2) to form methyl (2E)-4-(diethylphosphoryl)but-2-enoate (3) (3) at an approximately 80% yield.

Methyl (2E)-4-(diethylphosphoryl)but-2-enoate (3) was used directly in an HWE reaction with 4-formylphenyl acetate (4).

In a preferred variant, and in contrast to the known procedure from Li Y. et al., method step (b) in the method according to the present invention involves the use of sodium hydride at a temperature of −78° C.→0° C., preferably at a temperature of −58° C.→0° C., yielding methyl (2E, 4E)-5-(4-hydroxyphenyl)penta-2,4-dienoate (5; avenalumic acid methyl ester) at a decent yield and excellent purity, obtained by simple trituration.

Compared to the known synthesis, the intermediate product (5) can advantageously be purified by precipitation due to its polarity.

Starting with avenalumic acid methyl ester (5), the final deprotecting step is performed using 1M sodium hydroxide solution, yielding avenalumic acid (Avn Ac) at a quantitative yield and excellent purity, without further purification.

Compared to the known synthesis, this synthesis step can be performed under mild conditions.

For synthesising avenanthramide L (Avn L), the pure avenalumic acid (Avn Ac) was, surprisingly, reacted with 2-amino-5-hydroxybenzoic acid (6), using coupling reagents (hydroxybenzotriazole (HOBt) and 1-ethyl-3-(3-dimethylaminopropyl)carbodiimide (EDC) or 1-Ccano-2-ethoxy-2-oxoethylidenaminooxy)dimethylamino-morpholino-carbenium hexafluorophosphate (COMU)), without using any protecting groups.

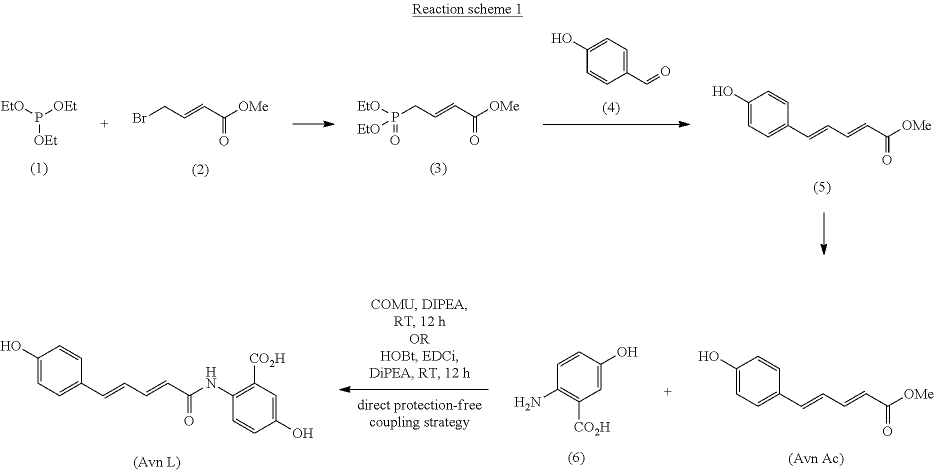

The present synthesis reduces the number of necessary steps by three as compared to the previously reported synthesis in Miyagawa, H et al., Bioscience, Biotechnology, Biochemistry 1995, 59(12), 2305-2306.

The use of coupling reagents and unprotected starting materials surprisingly results in faster, cheaper and environmentally friendlier synthesis, avoiding hazardous compounds such as thionyl chloride or oxalyl chloride. Purification of the avenanthramide L could be performed by aqueous separation followed by crystallisation, yielding decent purities, or by using preparative HPLC afterwards, resulting in excellent purities and yields, as demonstrated by Example 8 below.

While the invention has been specifically shown and described with reference to a preferred variant, it will be understood by those skilled in the art that various changes in form and detail may be made to it without departing from the spirit and scope of the invention. Moreover, the invention encompasses any combination of the elements described above, in all possible variations, unless specifically indicated otherwise.

The present invention shall now be described in detail with reference to the following examples, which are merely illustrative of the present invention, such that the content of the present invention is not limited by or to the following examples.

### EXAMPLES

Examples of the present invention are described below. The invention should not however be construed as being limited to the examples detailed.

### Example 1: NK1 Receptor Inhibition Study

The inhibition activity of avenanthramide L versus dihydroavenanthramide D and the structurally related avenanthramides A and D was evaluated in a radioligand binding assay.

The method employed in this study was adapted from the scientific literature so as to maximise reliability and reproducibility. Reference compound L-703,606 was run as an integral part of each experiment, to ensure the validity of the results obtained. The assay was performed under the conditions described in the following respective method.

Method:

### Tachykinin NK1

Source: Human recombinant CHO cells

### Vehicle: H₂O

Incubation time/temperature: 90 minutes at 4° C.
Incubation buffer: 20 mM HEPES, pH 7.4,
1 mM MnCl₂
0.1% BSA

### Kd: 2.10 nM

### Ligand: 0.80 nM [3H] Substance P

Non-specific ligand: 10.0 μM L-703,606 oxalate salt (CAS 144425-84-3)
Specific binding: 90%
Significance criteria: ≥50% of max. stimulation or inhibition
Bₘₐₓ: 1.70 pmol/mg protein

### LITERATURE REFERENCE

Patacchini R., Maggi C. A., Tachykinin receptors and receptor subtypes, Archives internationales de Pharmacodynamie et de Thérapie 1995, 329: 161-184

TABLE 2
Percent inhibition of specific binding to the tachykinin NK₁ receptor

| | Test concentration [ppm] | | | | | |
|---|---|---|---|---|---|---|
| Sample | 100 | 10 | | 1 | 0.1 | |
| Avn C | 94 | 27 | | 15 | 9 | |
| Avn L | 42 | 20 | | 15 | 9 | |
| Avn A | 21 | 14 | | 8 | 3 | |
| Dihydroavenanthramide D | 19 | 8 | | 6 | 8 | |
| Avenanthramide | CAS number | n | R1 | R2 | R3 | R4 |
| A | 108605-70-5 | 1 | OH | H | OH | H |
| C | 116764-15-9 | 1 | OH | OH | OH | H |
| L | 172549-38-1 | 2 | OH | H | OH | H |

Surprisingly, avenanthramide L with its one double bond more (n=2) than avenanthramide A (n=1) is twice as active at a test concentration of 100 ppm (42% versus 21% inhibition). Avenanthramide L is surprisingly also more active than the NK1 receptor antagonist dihydroavenanthramide D known from the literature. Avenanthramide C is approximately twice as active as avenanthramide L, but is highly unstable, whereas avenanthramide L is significantly less degradable, as can be seen from Example 2 below.

### Example 2: Stability Test of Different Avenanthramides in Solution

Stability under exposure to oxygen and temperature was evaluated for dissolved pure avenanthramides in aqueous ethanolic solution, both alone and as an avenanthramide mixture.

Avenantharmide mixtures used were DragoCalm® (Symrise; INCI Name: Aqua, Glycerin, Avena Sativa Kernel Extract) or DragoCalm® SP (Symrise; INCI Name: Aqua, Glycerin, Pentylene Glycol, Avena Sativa Kernel Extract).

The liquids were either exposed to 5 bars of oxygen at 70° C. for 24 hours using the Oxipress device or stored for 2 and 4 weeks at 40° C. in a heating cabinet.

The content of Avns was determined by HPLC, and the colour was measured by colorimetry (Hach Lange Lico 690 instrument) before and after treatment.

Color can be determined using the CIELAB color model which is based on an opponent color system. CIELAB indicates the color by values on three axes: L*, a*, and b* with dimension L for lightness and a* and b* for the color-opponent dimensions red/green and yellow/blue, based on nonlinearly compressed coordinates. The L* axis extends from black (0) to white (100), the a* axis from green (−a) to red (+a) and the b* axis from blue (−b) to yellow (+b).

The difference of 2 colors ΔE can be calculated using the following equation:

ΔE_{p,v}={square root over ((Lₚ*−Lᵥ*)²+(aₚ*−aᵥ*)²+bₚ*−bᵥ*)²)}

with p=sample 1 and v=sample 2

A difference of ΔE of 0.5-1 can be visually observed by a trained evaluator by naked eye. A difference of 2-4 can be observed visually also by a non-trained evaluator.

TABLE 3
Oxidative stability (Oxipress)

| | Content | | |
|---|---|---|---|
| Sample | Avn L [ppm] | Avn C [ppm] | Avn A [ppm] |

Avn L in aqueous ethanolic solution (0.01 wt %)

| | | | |
|---|---|---|---|
| Before treatment | 103 | — | — |
| After treatment | 100 | — | — |
| Degradation in % | 3 | — | — |
| ΔE | 0.6 | — | — |

Avn C in aqueous ethanolic solution (0.01 wt %)

| | | | |
|---|---|---|---|
| Before treatment | — | 105 | — |
| After treatment | — | 89 | — |
| Degradation in % | — | 15 | — |
| ΔE | — | 2.3 | — |

Avn A in aqueous ethanolic solution (0.01 wt %)

| | | | |
|---|---|---|---|
| Before treatment | — | — | 104 |
| After treatment | — | — | 104 |
| Degradation in % | — | — | 0 |
| ΔE | — | — | 0.2 |

Solution of Avn mixture in glycerin/water as commercially
available from Symrise (trade name: DragoCalm ® SP)

| | | | |
|---|---|---|---|
| Before treatment | 8 | 10 | 35 |
| After treatment | 7 | <1 | 34 |
| Degradation in % | 13 | 100 | 3 |

| | |
|---|---|
| ΔE | 7.1 |

The results clearly show that the best NK1 receptor inhibitor, Avn C, is also the most unstable, whereas Avn A, which is stable, is significantly less active. Avn L is only half as effective as Avn C, but clearly much more stable.

The avenanthramide mixture confirms these results. Avenanthramide C is completely degraded after 24 hours of oxygen exposure, while the Avn L content is only reduced by 13%. Avenanthramide A, which is less biologically active, is stable under these conditions.

TABLE 4
Temperature stability at 40° C.

| | Content | | |
|---|---|---|---|
| Sample | Avn C [ppm] | Avn A [ppm] | Avn L [ppm] |

Solution of Avn mixture in glycerin/water as commercially
available from Symrise (trade name: DragoCalm ®)

| | | | |
|---|---|---|---|
| Before treatment | 20 | 50 | 11 |
| After 2 weeks at 40° C. | 16 | 50 | 11 |
| degradation in % | 20 | 0 | 0 |
| After 4 weeks at 40° C. | 10 | 50 | 10 |
| degradation in % | 50 | 0 | 9 |

| | |
|---|---|
| ΔE | 3.1 |

The results clearly show that the least effective NK1R inhibitor Avn A is the most stable (no degradation after 2 and 4 weeks at 40° C.), followed by Avn L (no and only 9% degradation after 2 and 4 weeks). Avn C, the most potent NK1R inhibitor, is also the least stable when exposed to higher temperature with 20% degradation after 2 weeks and 50% after 4 weeks.

### Example 3: Effect of Avenanthramides on the Expression of Heat Shock Proteins in Human Keratinocytes

Neonatal human epidermal keratinocytes (nHEK) were cultivated in an EpiLife® medium (Gibco) including an HKGS kit (Gibco) with 5% C02 at 37° C. in accordance with the supplier's instructions.

The cells were treated for 24 hours, with the test compounds dissolved in DMSO and DMSO alone as the vehicle control. Genomic target expression levels in treated cells were measured using a quantitative Real-Time PCR comparison to vehicle control treatment.

RNA was isolated using Qiagen's RNeasy® Mini Kit. The total RNA concentrations were measured using Eppendorf's pCuvetteG 1.0 and BioPhotometer, by measuring the absorption at 260 nm. Purity control values such as E260/280 and E260/230 were calculated simultaneously. Reverse transcription was performed using the high-capacity RNA-to-cDNA kit of Applied Biosystems, in accordance with the supplier's instructions. Samples were treated in Biometra's PCR Thermocycler.

For fast real-time PCR, the cDNA was diluted with RNase-free water, and the TaqMan™ Fast Universal PCR Master Mix of Applied Biosystems was used.

Quantitative real-time PCR was performed using the StepOnePlus fast real-time PCR instrument by Applied Biosystems. Analysis was conducted using the StepOne software and 2^{-ΔΔct} method (normalised to endogenous control HTRP1 expression).

For upregulations, RQ values ≥2.0 are considered to be relevant.

TABLE 5
Results

RQ value

| Gene | 100 μM Avn L | 100 μM Avn L |
|---|---|---|
| HPRT1 (housekeeping) | 1.0 | 1.0 |
| HSPB2 (=HSP27) | 5.0 | 1.9 |
| CRYAB | 2.1 | 1.6 |
| HSP90AA1 | 0.9 | 0.9 |
| HSP90AB1 | 0.4 | 0.5 |

The results show that avenanthramide L at 100 μM upregulates the small heat shock proteins HSPB2 (=HSP27) and CRYAB (=αB-crystallin), but has no effect on the large heat shock proteins HSP90AA1 and HSP90AB1.

Unlike avenanthramide L, avenanthramide A does not result in a relevant upregulation of small HSPs, or only in a clearly less effective way (RQ values of 5.0 vs 1.9 for modulation of HSPB2 gene expression) when tested at the same test concentration of 100 μM.

### Example 4: Gene Expression of Keratinocytes

Neonatal human epidermal keratinocytes (nHEK) were cultivated and treated with the test compounds for 24 hours, following which fast real-time PCR was performed as described in Example 3 using another customised gene array with different genes.

TABLE 6
Results for modulation of gene expression

RQ value

| Gene | 100 μM Avn L | 100 μM Avn L |
|---|---|---|
| HPRT1 (housekeeping) | 1.0 | 1.0 |
| BLVRB | 4.1 | 1.0 |
| CD44 | 2.1 | 1.0 |

The results show that avenanthramide L at 100 μM upregulates BLVRB and CD 44, while avenanthramide A has no effect at the same test concentration.

### Example 5: Radical-Scavenging Activity (ABTS Assay)

With the aid of the ABTS assay, the antioxidative capacity of Avn L and Avn A were evaluated and compared.

2,2′-azino-bis-(3-ethylbenzothiazoline-6-sulphonic acid) (ABTS) was transformed into the blue-green radical cation ABTS^{•+} using potassium persulphate. Through the addition of [6]-paradol and alpha-tocopherol, the radical cations were reduced and discoloration was observed, as determined photometrically by absorption at 734 nm. Inhibition of radical formation in the test substance is calculated by the following formula:

$$Inhibition [ \% ] = 100 - ( \frac{{A}_{test substance}}{{A}_{c ontrol}} \times 100 )$$

where
A_{test substance} is the absorption in the wells with the test substance including [6]-paradol and alpha-tocopherol
A_{control} is the absorption in the wells with no test substance.

Values for IC50 (the concentration at which radical formation is inhibited by 50%) were calculated from the inhibition of radical formation [%] in a series of dilutions of tested samples. The results are shown in Table 7.

TABLE 7
Results

Sample

| Mean antioxidant activity | Avn L | Avn A |
|---|---|---|
| 5 μM | 40 ± 4 | 40 ± 4 |
| 10 μM | 74 ± 4 | 76 ± 4 |
| IC50 | 0.0045 ± 0.0004 | 0.0042 ± 0.0003 |

The results clearly show than Avn L has almost the same antioxidant activity at almost the same concentration as Avn A.

### Example 6: Cellular Antioxidant Activity (DCF-DA Assay)

Primary human dermal fibroblasts were seeded in a 96-well microtiter plate at a concentration of 0.5×10⁴ cells/well. Cultivation took place at 37° C. and 5% CO₂ in DMEM, enriched with 10% foetal calf serum. Confluence was supposed to be around 70% at the time, the incubation with the test substances began. The test substances were applied to the cells at a concentration of 500 μM. After 24 h of incubation, 100 μL H₂DCF-DA-solution (10 μM) incl. DAPI (1:1000) was added to all samples (excluded the background-control) and incubated for one hour to deesterify the H₂DCF-DA by cellular esterases. The resulting H₂DCF was thereby trapped inside the cell. After the incubation, the cells were washed and the prooxidant challenge was set (1 mM, 1 h). The resulting fluorescence was read at λₑₓ 504 nm; λₑₘ 524 nm. An increased level of ROS (reactive oxygen species) led to an increased amount of fluorescence.

The inhibition of the oxidation in the presence of test substances was calculated according to the following equation:

$$Inhibition of oxidation [ \% ] = 100 - ( \frac{R F {U}_{test substance} - R F {U}_{without cells}}{R F {U}_{c o n t r o l} - R F {U}_{w i t hout cells}} \times 100 )$$

The abbreviations have the following meanings:
RFU test substance: Relative fluorescence units of the wells with test substance and with cells
RFU control: Relative fluorescence units of the wells without test substance, but with cells
RFU without cells: Relative fluorescence units of the wells without test substance and without cells (blank)

TABLE 8
Results

Sample

| Inhibition of oxidation | Avn L | Avn A |
|---|---|---|
| 500 μM | 49 ± 7% | 14 ± 6% = not active |

The results clearly show that in the cellular system, avenanthramide L exhibits a higher antioxidant activity than avenanthramide A at the same test concentration of 500 μM.

### Example 7: NK1 Receptor Inhibition Study for Synergism

In another experiment, the inhibitory activity of a combination of avenanthramide L and another avenanthramide was evaluated in comparison to both substances alone in the radioligand binding assay as described in Example 1 to investigate a potential synergism.

In the connection of this text, synergistic action is to be understood as meaning an action which is increased beyond the additive action of the compounds displaying synergy. This can be recorded by the synergy index (SI) value according to Kull (D. C. Steinberg, Cosmetics & Toiletries 2000, 115 (11), 59-62 and F. C. Kull et al., Applied Microbiology 1961, 9, 538-541). Substance combinations in which both components display the synergistically increased action, and also substance combinations in which only one component displays the synergistically increased action, while the other component acts merely as an intensifier (booster), fall under the given definition of the synergy effect.

The synergy index (SI) values according to Kull for the tachykinin NK1 receptor inhibition for a combination of Avn L and Avn A was calculated as follows:

SI=C×l/L+C×a/A with Kull's equation:

C=inhibition of the combination
L=inhibition of Avn L
A=inhibition of Avn A
l=proportional factor for Avn L in the mixture=0.2
a=proportional factor of Avn A in the mixture=0.8

TABLE 9
Percent inhibition of specific binding to the tachykinin
NK1 receptor and calculated synergy index

| | Test concentration [ppm] |
|---|---|
| Sample | 125 |
| Avn A | 9 |
| Avn L | 37 |
| Avn A:Avn L = 4:1 (80%:20%) | 27 |
| SI | 2.546 |

The experiment confirms again the superior activity of Avn L versus Avn A.

Evidence of a synergy effect results from SI values of >1 as then the inhibition of the combination is stronger than the proportional individual contributions of the two Avns alone.

The SI of 2.546 clearly shows that Avn L and Avn A display a synergistically increased inhibitory action. A synergistic combination of active compounds has the advantage that overall less active compound is required to achieve the particular action.

### Example 8: Extraction of Non-Milled Naked Oat (Avena nuda) Grains with Different Extractants

100 g naked oat grains (bought from Bohlenser Mühle, cultivated in Germany, cultivar Oliver) were extracted with 300 g of extractant as given in the following table (w/w) for 2 hours at 55° C. under stirring. The mixture was cooled down to room temperature and the grains were separated from the extract solution by centrifugation and filtration. The extracted grains were extracted with a second portion of 300 g extractant again for 2 h at 55° C. and extract solution was separated from grains as described above. The two extract solutions were combined, the extracting solvents were removed under vacuum by use of an evaporator and the obtained dry extracts were weight to determine the extraction yields. Avns were quantified in the dry extract by HPLC using an acetonitrile/water/0.1% formic acid gradient on an ODS-AQ column (YMC) at 330 nm.

TABLE 10
Characterization of naked oat extracts obtained with different extractants

| | | Content | | | | | |
|---|---|---|---|---|---|---|---|
| | Dry | | | | Sum | | |
| | extract | | | | Avns | | Avn L |
| Extractant | yield* | Avn C | Avn A | Avn B | Ato C | Avn L | iso** |
| (w/w) | [wt.-%] | [ppm] | [ppm] | [ppm] | [ppm] | [ppm] | [ppm] |
| Water | 10.3 | n.d. | 12 | 14 | 26 | n.d. | n.d. |
| Methanol/ | 2.2 | 110 | 737 | 1183 | 2030 | 128 | 105 |
| water 3:7 | | | | | | | |
| Methanol/ | 2.6 | 364 | 1029 | 1471 | 2864 | 276 | 195 |
| water 1:1 | | | | | | | |
| Methanol/ | 2.3 | 349 | 967 | 1441 | 2757 | 311 | 194 |
| water 7:3 | | | | | | | |
| Ethanol/ | 3.0 | 373 | 1051 | 1471 | 2895 | 328 | 188 |
| water 1:1 | | | | | | | |
| Ethanol/ | 2.9 | 61 | 157 | 534 | 752 | 5 | 35 |
| water 1:4 | | | | | | | |
| Isopropanol/ | 3.4 | 471 | 1118 | 1673 | 3262 | 347 | 227 |
| water 3:7 | | | | | | | |
| Isopropanol/ | 3.1 | 423 | 1049 | 1535 | 3007 | 353 | 231 |
| water 1:1 | | | | | | | |
| Isopropanol/ | 2.4 | 492 | 1151 | 1639 | 3282 | 422 | 216 |
| water 7:3 | | | | | | | |
| Acetone/ | 3.0 | 344 | 1080 | 1655 | 3079 | 265 | 220 |
| water 3:7 | | | | | | | |
| Acetone/ | 3.7 | 437 | 1063 | 1539 | 3039 | 270 | 194 |
| water 1:1 | | | | | | | |
| Acetone/ | 2.6 | 627 | 1527 | 2194 | 4348 | 423 | 287 |
| water 7:3 | | | | | | | |

*Based on oat grains
**Structural isomer of Avn L with same molecular weight and fragmentation pattern according to HPLC-MS measurement, quantified by HPLC as Avn L
n.d. = not detectable
n.a. = not analyzed

### Example 9: Formulation Examples

In the formulation examples 1 to 11 the following two perfume oils PFO1 and PFO2 were each used as fragrance (DPG=dipropylene glycol).

TABLE 11
Perfume oil PFO1 with rose smell (amounts in parts by weight)

| Component | Amount |
|---|---|
| Acetophenone, 10% in DPG | 10.00 |
| n-Undecanal | 5.00 |
| Aldehyde C14, so-called (peach aldehyde) | 15.00 |
| Allylamyl glycolate, 10% in DPG | 20.00 |
| Amyl salicylate | 25.00 |
| Benzyl acetate | 60.00 |
| Citronellol | 80.00 |
| d-Limonene | 50.00 |
| Decenol trans-9 | 15.00 |
| Dihydromyrcenol | 50.00 |
| Dimethylbenzylcarbinyl acetate | 30.00 |
| Diphenyloxide | 5.00 |
| Eucalyptol | 10.00 |
| Geraniol | 40.00 |
| Nerol | 20.00 |
| Geranium oil | 15.00 |
| Hexenol cis-3, 10% in DPG | 5.00 |
| Hexenyl salicylate cis-3 | 20.00 |
| Indole, 10% in DPG | 10.00 |
| Alpha-ionone | 15.00 |
| Beta-ionone | 5.00 |
| Lilial ® (2-methyl-3-(4-tert-butyl-phenyl)propanal) | 60.00 |
| Linalool | 40.00 |
| Methylphenyl acetate | 10.00 |
| Phenylethyl alcohol | 275.00 |
| Styrolyl acetate | 20.00 |
| Terpineol | 30.00 |
| Tetrahydrolinalool | 50.00 |
| Cinnamyl alcohol | 10.00 |
| Total: | 1,000.00 |

TABLE 12
Perfume oil PFO2 with white blossom and
musk smell (amounts in parts by weight)

| Component | Amount |
|---|---|
| Benzyl acetate | 60.00 |
| Citronellyl acetate | 60.00 |
| Cyclamenaldehyde (2-methyl-3-(4-isopropylphenyl)propanal | 20.00 |
| Dipropylene glycol (DPG) | 60.00 |
| Ethyllinalool | 40.00 |
| Florol (2-isobutyl-4-methyltetrahydro-2H-pyran-4-ol) | 30.00 |
| Globanone ® [(E/Z)-8-cyclohexadecen-1-one] | 180.00 |
| Hedione ® (methyldihydrojasmonate) | 140.00 |
| Hexenyl salicylate, cis-3 | 10.00 |
| Vertocitral (2,4-dimethyl-3-cyclohexenecarboxaldehyde) | 5.00 |
| Hydratropaaldehyde, 10% in DPG | 5.00 |
| Isodamascone (1-(2,4,4-trimethyl-2-cyclohexen-1-yl)-2- | 5.00 |
| buten-1-one, 10% in DPG | |
| Isomuscone (cyclohexadecanone) | 40.00 |
| Jacinthaflor (2-methyl-4-phenyl-1,3-dioxolane) | 10.00 |
| Cis-jasmone, 10% in DPG | 20.00 |
| Linalool | 50.00 |
| Linalyl acetate | 30.00 |
| Methyl benzoate, 10% in DPG | 25.00 |
| para-Methyl cresol, 10% in DPG | 10.00 |
| Nerol | 20.00 |
| Phenylpropylaldehyde | 5.00 |
| 2-Phenylethyl alcohol | 82.00 |
| Tetrahydrogeraniol | 13.00 |
| 2,2-Dimethyl-3-cyclohexyl-1-propanol | 80.00 |
| Total: | 1,000.00 |

TABLE 13
Cosmetic formulations (amounts in parts by weight)

| Ingredients | 1 | 2 | 3 | 4 | 5 | 6 | 7 | 8 | 9 | 10 | 11 |
|---|---|---|---|---|---|---|---|---|---|---|---|
| Avenanthramide L | 0.1 | 0.0001 | | 0.01 | | 0.005 | | | 0.003 | 0.0005 | |
| Avenanthramide A | | 0.0004 | | | | | | | | 0.002 | |
| Oat kernel extract in | | | 3 | 1 | | | 5 | | | 2.5 | |
| glycerin/water | | | | | | | | | | | |
| standardized to | | | | | | | | | | | |
| 10-50 ppm Avn L | | | | | | | | | | | |
| Water (Aqua), | | | | | | | | | | | |
| Glycerin, Avena Nuda | | | | | | | | | | | |
| (Oat) Kernel Extract | | | | | | | | | | | |
| Spray-dried oat | | | | | 3 | | | 1 | | | 2 |
| straw extract on | | | | | | | | | | | |
| maltodextrin | | | | | | | | | | | |
| standardized to ≥ | | | | | | | | | | | |
| 100 ppm Avn L | | | | | | | | | | | |
| Maltodextrin, Avena | | | | | | | | | | | |
| Sativa (Oat) Extract | | | | | | | | | | | |
| Actipone ® Laminaria | | | | | | 0.3 | | | | | |
| Saccharina | | | | | | | | | | | |
| Glycerin, Water | | | | | | | | | | | |
| (Aqua), Laminaria | | | | | | | | | | | |
| Saccharina Extract | | | | | | | | | | | |
| Allantoin | 0.1 | | | | | 0.1 | | | | | |
| Allantoin | | | | | | | | | | | |
| Aloe Vera Gel | | | | | | 1 | | | | | |
| Conc. 10:1 | | | | | | | | | | | |
| Aloe | | | | | | | | | | | |
| Barbadensis(Aloe) | | | | | | | | | | | |
| Leaf Juice | | | | | | | | | | | |
| Aluminium Stearat | | | | 1.2 | | | | | | | |
| Aluminium Stearate | | | | | | | | | | | |
| β-Arbutin | | | | | | | | | 1 | | |
| Arbutin | | | | | | | | | | | |
| Arlypon ® F | | | | | | | 2 | | | | |
| Laureth-2 | | | | | | | | | | | |
| Avocado Oil | | | 3 | | | | | | | | |
| Persea Gratissima | | | | | | | | | | | |
| (Avocado) Oil | | | | | | | | | | | |
| Betulin 90% | | | | | | | | | | 0.1 | |
| Betulin | | | | | | | | | | | |
| Biotive L-Arginine | | 0.6 | | | | | | | | | 0.5 |
| Arginine | | | | | | | | | | | |
| Biotive Troxerutin | | 0.5 | | | | | | | | | 0.5 |
| Troxerutin | | | | | | | | | | | |
| (−)-alpha-Bisabolol | | | | | | | | | | | 0.1 |
| Bisabolol | | | | | | | | | | | |
| Carbopol Aqua SF-1 | | | | | 5 | | | | | | |
| Polymer | | | | | | | | | | | |
| Acrylates Copolymer | | | | | | | | | | | |
| Carbopol ® Ultrez-10 | | 0.2 | | | | 0.4 | | | 0.2 | | |
| Carbomer | | | | | | | | | | | |
| CeramideBIO ® | | | | | | | | | | 0.5 | |
| Cetylhydroxyproline | | | | | | | | | | | |
| Palmitamide | | | | | | | | | | | |
| Citric acid | | | | | 0.2 | | 0.5 | | | | |
| 10% in Water | | | | | | | | | | | |
| Covi-Ox ® T-70 | | | 0.1 | | | | | | | | |
| Tocopherol | | | | | | | | | | | |
| Crinipan ® AD | | | | | | | 0.3 | | | | |
| Climbazole | | | | | | | | | | | |
| Cutina ® PES | | 2 | | | | | | | | | |
| Pentaerythrityl | | | | | | | | | | | |
| Distearate | | | | | | | | | | | |
| D-Panthenol | 1 | | 1 | | | 0.5 | 0.5 | | | | |
| Panthenol | | | | | | | | | | | |
| Dehyton K | | | | | 8 | | 8 | | | | |
| Cocamidopropyl | | | | | | | | | | | |
| Betaine | | | | | | | | | | | |
| Dermacryl ® AQF | | | | | | | | | | | 2 |
| Acrylates Copolymer | | | | | | | | | | | |
| Dow Corning | 2 | 2 | | | | | | | 0.5 | 0.5 | |
| 200 (100 cs) | | | | | | | | | | | |
| Silicone Fluid | | | | | | | | | | | |
| Dimethicone | | | | | | | | | | | |
| Dow Corning 246 | | | 2 | | | | | | | | 3 |
| Fluid | | | | | | | | | | | |
| Cyclohexasiloxane, | | | | | | | | | | | |
| Cyclopentasiloxane | | | | | | | | | | | |
| Dracorin ® CE | | | | | | | | | | 1.5 | |
| Glyceryl Stearate | | | | | | | | | | | |
| Citrate | | | | | | | | | | | |
| Dracorin ® GMS | | | | | | | | | | 2 | |
| Glyceryl Stearate | | | | | | | | | | | |
| Dracorin ® GOC | | | 2 | | | | | | | | |
| lyceryl Oleate Citrate, | | | | | | | | | | | |
| Caprylic/Capric | | | | | | | | | | | |
| Triglyceride | | | | | | | | | | | |
| Dragocid ® Liquid | | | | | | | | | 0.8 | | |
| Phenoxyethanol, | | | | | | | | | | | |
| Methyl-paraben, | | | | | | | | | | | |
| Ethylparaben, | | | | | | | | | | | |
| Butylparaben, | | | | | | | | | | | |
| Propylparaben, | | | | | | | | | | | |
| Isobutylparaben | | | | | | | | | | | |
| Dragoderm ® | | | | | | | 0.5 | | | | |
| Glycerin, Triticum | | | | | | | | | | | |
| Vulgare (Wheat) | | | | | | | | | | | |
| Gluten, Water (Aqua) | | | | | | | | | | | |
| Dragosan ® W/OP | | | | 8 | | | | | | | |
| Sorbitan Isostearate, | | | | | | | | | | | |
| Hydrogenated Castor | | | | | | | | | | | |
| Oil, Ceresin, Beeswax | | | | | | | | | | | |
| (Cera Alba) | | | | | | | | | | | |
| Dragosantol ® 100 | | | | | 0.2 | | | | | | |
| Bisabolol | | | | | | | | | | | |
| Dragosine ® | | 0.2 | | | | | | | | | |
| Carnosine | | | | | | | | | | | |
| Dragoxat ® 89 | | 5 | | 7 | | | | | | 2 | 2 |
| Ethylhexyl | | | | | | | | | | | |
| Isononanoate | | | | | | | | | | | |
| Disodium EDTA | 0.1 | 0.1 | | | | | | | 0.1 | | 0.1 |
| Emulsiphos ® | | 2 | | | | | | | 1.5 | 2 | 2 |
| Potassium Cetyl | | | | | | | | | | | |
| Phosphate, | | | | | | | | | | | |
| Hydrogenated Palm | | | | | | | | | | | |
| Glycerides | | | | | | | | | | | |
| Ethanol | | | 5 | | | 8 | | | | | |
| Extrapone ® Aloe vera | | | | | | 2 | | | | | |
| Water (Aqua), Aloe | | | | | | | | | | | |
| Barbadensis, | | | | | | | | | | | |
| Propylene Glycol, | | | | | | | | | | | |
| Alcohol | | | | | | | | | | | |
| Extrapone ® Witch | 1 | | | | | | | | | | |
| Hazel | | | | | | | | | | | |
| Propylene Glycol, | | | | | | | | | | | |
| Hamamelis Virginiana | | | | | | | | | | | |
| (Witch Hazel) Water, | | | | | | | | | | | |
| Water (Aqua), | | | | | | | | | | | |
| Hamamelis Virginiana | | | | | | | | | | | |
| (Witch Hazel) Extract | | | | | | | | | | | |
| Extrapone ® | | | | | | | 0.3 | | | | |
| Rosemary | | | | | | | | | | | |
| Glycerin, Water | | | | | | | | | | | |
| (Aqua), Rosmarinus | | | | | | | | | | | |
| Officinalis (Rosemary) | | | | | | | | | | | |
| Leaf Extract | | | | | | | | | | | |
| Extrapone ® | | | | | | 0.5 | | | | | |
| Seaweed Water | | | | | | | | | | | |
| (Aqua), Butylene | | | | | | | | | | | |
| Glycol, Fucus | | | | | | | | | | | |
| Vesiculosus Extract | | | | | | | | | | | |
| Farnesol DT | | | | | | | | 0.2 | | | |
| Phenoxyethanol, | | | | | | | | | | | |
| Farnesol, Bisabolol | | | | | | | | | | | |
| Food colour brown | | 2 | | | | | | | | | |
| E172 + E171 Powder | | | | | | | | | | | |
| Frescolat ® MGA | | | 0.1 | | | | | | | | |
| Menthone Glycerin | | | | | | | | | | | |
| Acetal | | | | | | | | | | | |
| Frescolat ® ML | | | 0.5 | | | 0.3 | 0.2 | | | | |
| Menthyl Lactate | | | | | | | | | | | |
| Frescolat ® X-Cool | | | | | | | | | | 0.2 | |
| Menthyl Ethylamido | | | | | | | | | | | |
| Oxalate | | | | | | | | | | | |
| Genapol ® LRO | | | | | | | 37 | | | | |
| Liquid Sodium | | | | | | | | | | | |
| Laureth Sulfate | | | | | | | | | | | |
| Givobio ® GZN | | | | | | | | | | 0.5 | |
| Zinc Gluconate | | | | | | | | | | | |
| Glycerin | 1.5 | | 4 | 3 | | | | | 3.5 | 3 | 3 |
| Hydrolite ® 5 | 3 | | 5 | | 2 | 5 | | 5 | | | 2 |
| Pentylene Glycol | | | | | | | | | | | |
| Hydroviton-24 ® | | | | 1 | | | | | | | |
| Water (Aqua), | | | | | | | | | | | |
| Pentylene Glycol, | | | | | | | | | | | |
| Glycerin, Lactic Acid, | | | | | | | | | | | |
| Sodium Lactate, | | | | | | | | | | | |
| Serine, Urea, Sorbitol, | | | | | | | | | | | |
| Sodium Chloride, | | | | | | | | | | | |
| Allantoin | | | | | | | | | | | |
| Hydroviton ® | | | | | | | | 1 | | 2 | |
| Plus 2290 | | | | | | | | | | | |
| Water (Aqua), | | | | | | | | | | | |
| Pentylene Glycol, | | | | | | | | | | | |
| Glycerin, Fructose, | | | | | | | | | | | |
| Urea, Citric acid, | | | | | | | | | | | |
| Sodium Hydroxide, | | | | | | | | | | | |
| Maltose, Sodium | | | | | | | | | | | |
| PCA, Sodium | | | | | | | | | | | |
| Chloride, Sodium | | | | | | | | | | | |
| Lactate, Trehalose, | | | | | | | | | | | |
| Allantoin, Sodium | | | | | | | | | | | |
| Hyaluronate, Glucose | | | | | | | | | | | |
| Isoadipate | | | | | | | | | 2 | | |
| Diisopropyl Adipate | | | | | | | | | | | |
| Isodragol ® | 1 | | | | | | | | | 3 | 2 |
| Triisononanoin | | | | | | | | | | | |
| Jojoba Oil | | | | 2 | | | | | | | |
| Simmondsia | | | | | | | | | | | |
| Chinensis (Jojoba) | | | | | | | | | | | |
| Seed Oil | | | | | | | | | | | |
| Potassium sorbate | | | 0.1 | | | | | | | | |
| Keltrol ® CG-RD | | 0.2 | | | | | | | 0.2 | | 0.4 |
| Xanthan Gum | | | | | | | | | | | |
| Kojic acid | | | | | | | | | 0.5 | | |
| Kojic acid | | | | | | | | | | | |
| Lanette ® 16 | | | | | | | | | 1.5 | | 1 |
| Cetyl Alcohol | | | | | | | | | | | |
| Lanette ® O | | | | | | | | | | 2 | 0.5 |
| Cetearyl Alcohol | | | | | | | | | | | |
| Lara Care ® A-200 | | | | | | | | | | | 0.3 |
| Galactoarabinan | | | | | | | | | | | |
| Locron ® L | | | | | | | | 16 | | | |
| Aluminium | | | | | | | | | | | |
| Chlorohydrate | | | | | | | | | | | |
| Magnesiumsulfate | | | | 0.7 | | | | | | | |
| Mineral Oil | | | | 8 | | | | | | | |
| Sodium | | | | | | | | | 1 | | |
| ascorbylphosphate | | | | | | | | | | | |
| Sodium chloride | | | | | | | 0.1 | | | | |
| Sodiium hydroxide | 1 | | | | 2 | 0.7 | | | 0.2 | 0.3 | |
| 10% in Water | | | | | | | | | | | |
| Neo Heliopan ® 303 | | 4 | | | | | | | | | 10 |
| Octocrylene | | | | | | | | | | | |
| Neo Heliopan ® 357 | | 2 | | | | | | | 2 | | 3 |
| Butylmethoxydibenzoyl- | | | | | | | | | | | |
| methane | | | | | | | | | | | |
| Neo Heliopan ® AP, | | 6.7 | | | | | | | | | 6.7 |
| 15% solution, | | | | | | | | | | | |
| neutralised with | | | | | | | | | | | |
| L-Arginin | | | | | | | | | | | |
| Aqua, Disodium | | | | | | | | | | | |
| Phenyl | | | | | | | | | | | |
| Dibenzimidazole | | | | | | | | | | | |
| Tetrasulfonate, | | | | | | | | | | | |
| Arginine | | | | | | | | | | | |
| Neo Heliopan ® AV | | | | | | | | | 7.5 | | |
| Ethylhexyl | | | | | | | | | | | |
| Methoxycinnamate | | | | | | | | | | | |
| Neo Heliopan ® BB | | | | | | | | | 3 | | |
| Benzophenone-3 | | | | | | | | | | | |
| Neo Heliopan ® E | | | | | | | | | | | 1 |
| 1000 Isoamyl | | | | | | | | | | | |
| p. Methoxycinnamate | | | | | | | | | | | |
| Neo Heliopan ® HMS | | | | | | | | | 10 | | 5 |
| Homosalate | | | | | | | | | | | |
| Neo Heliopan ® OS | | 3 | | | | | | | 5 | | |
| Ethylhexyl Salicylate | | | | | | | | | | | |
| Neo Heliopan ® | | 10 | | | | | | | | | 10 |
| Hydro, | | | | | | | | | | | |
| 20% solution, | | | | | | | | | | | |
| neutralized with | | | | | | | | | | | |
| Biotive Arginine, | | | | | | | | | | | |
| Aqua, | | | | | | | | | | | |
| Phenylbenzimidazole, | | | | | | | | | | | |
| Sulphonic Acid, | | | | | | | | | | | |
| Arginin | | | | | | | | | | | |
| Neo-PCL Water | | | | | | 1 | 1.5 | 2 | | | |
| Soluble N | | | | | | | | | | | |
| Trideceth-9, PEG-5 | | | | | | | | | | | |
| Ethylhexanoate, | | | | | | | | | | | |
| Water (Aqua) | | | | | | | | | | | |
| Neutral Oil | | | 5 | | | | | | | 10 | |
| Caprylic/Capric | | | | | | | | | | | |
| Triglyceride | | | | | | | | | | | |
| Niacinamide | | | | | 1 | | | | | | |
| Ozokerite Wax 2389 | | | | 2 | | | | | | | |
| Ozokerite | | | | | | | | | | | |
| Parfum oil PFO1 or | 0.05 | 0.3 | 0.25 | 0.3 | | 0.1 | 0.5 | 0.7 | 0.3 | 0.1 | 0.2 |
| PFO2 | | | | | | | | | | | |
| Parfum | | | | | | | | | | | |
| PCL-Liquid 100 | 3 | 2 | 4 | 5 | | | | | | | |
| Cetearyl | | | | | | | | | | | |
| Ethylhexanoate | | | | | | | | | | | |
| PCL-Solid | 1 | | 0.5 | | | | | | | | |
| Stearyl Heptanoate, | | | | | | | | | | | |
| Stearyl Caprylate | | | | | | | | | | | |
| Pemulen ® TR-2 | 0.6 | | 0.25 | | | | | | | | |
| Acrylates/C 10-30 | | | | | | | | | | | |
| Alkyl Acrylate | | | | | | | | | | | |
| Crosspolymer | | | | | | | | | | | |
| Phenethylalkohol | | | | | 0.2 | | | | | | |
| Phenoxyethanol | | | | | | | | | | | 0.2 |
| Phytoconcentrole ® | 1 | | | | | | | | | | |
| Shea Butter | | | | | | | | | | | |
| Glycine Soja | | | | | | | | | | | |
| (Soybean) Oil, | | | | | | | | | | | |
| Butyrospermum Parkii | | | | | | | | | | | |
| (Shea Butter) | | | | | | | | | | | |
| Polymer JR 400 | | | | | | | 0.4 | | | | |
| Polyquaternium-10 | | | | | | | | | | | |
| Propylenglycol-1,2 | | | | | | 5 | | 3 | | | |
| Propylene Glycol | | | | | | | | | | | |
| Silcare Silicone | | | | | | | | | | | 1 |
| 41M65 Stearyl | | | | | | | | | | | |
| Dimethicone | | | | | | | | | | | |
| Solubilizer | | | | | | | | 3 | | | |
| PEG-40 | | | | | | | | | | | |
| Hydrogenated Castor | | | | | | | | | | | |
| Oil, Trideceth-9, | | | | | | | | | | | |
| Propylene Glycol, | | | | | | | | | | | |
| Water (Aqua) | | | | | | | | | | | |
| Sulfetal LA | | | | | 12 | | | | | | |
| Ammonium Lauryl | | | | | | | | | | | |
| Sulfate | | | | | | | | | | | |
| SymCalmin ® | 1 | | | | | | 0.1 | | | | |
| Butylene Glycol, | | | | | | | | | | | |
| Pentylene Glycol, | | | | | | | | | | | |
| Hydroxyphenyl | | | | | | | | | | | |
| Propamidobenzoic | | | | | | | | | | | |
| Acid | | | | | | | | | | | |
| SymClariol ® | | 0.5 | | | 1 | | | | 0.3 | | |
| Decylene Glycol | | | | | | | | | | | |
| SymDecanox HA | | | 2 | | | | | | | | |
| Caprylic/Capric | | | | | | | | | | | |
| Triglyceride, | | | | | | | | | | | |
| Hydroxymethoxyphenyl | | | | | | | | | | | |
| Decanone | | | | | | | | | | | |
| SymDeo ® B125 | | | | | | | | 0.2 | | | |
| 2-Methyl 5- | | | | | | | | | | | |
| Cyclohexylpentanol | | | | | | | | | | | |
| SymDeo ® MPP | | | | | | | | 0.5 | | | |
| Dimethyl Phenyl 2- | | | | | | | | | | | |
| Butanol | | | | | | | | | | | |
| Symdiol ® 68 | 1 | 0.5 | | | | | | | | | 0.3 |
| 1.2-Hexanediol, | | | | | | | | | | | |
| Caprylyl Glycol | | | | | | | | | | | |
| SymFinity ® 1298 | | | | 0.05 | | | | | | | |
| Echinacea Purpurea | | | | | | | | | | | |
| Extract | | | | | | | | | | | |
| SymGlucan ® | 1 | | 5 | | | | | | | | 2 |
| Water (Aqua), | | | | | | | | | | | |
| Glycerin, β-Glucan | | | | | | | | | | | |
| SymHair ® Force | | | | | | | 2 | | | | |
| 1631 | | | | | | | | | | | |
| Pentylene Glycol, | | | | | | | | | | | |
| Isochrysis galbana | | | | | | | | | | | |
| Extract | | | | | | | | | | | |
| SymHelios ® 1031 | | 0.5 | | | | | | | | | |
| Benzylidene | | | | | | | | | | | |
| Dimethoxydimethylindanone | | | | | | | | | | | |
| SymLift | | 2 | | | | | | | | | |
| Water, trehalose, | | | | | | | | | | | |
| glycerin, pentylene | | | | | | | | | | | |
| glycol, β-glucan, | | | | | | | | | | | |
| hordeum vulgare seed | | | | | | | | | | | |
| extract, sodium | | | | | | | | | | | |
| hyaluronate, 1,2- | | | | | | | | | | | |
| Hexanediol, caprylyl | | | | | | | | | | | |
| glycol, sodium | | | | | | | | | | | |
| benzoate, | | | | | | | | | | | |
| maltodextrine | | | | | | | | | | | |
| SymMatrix | | 0.1 | 0.3 | | | | | | | | |
| Maltodextrin, Rubus | | | | | | | | | | | |
| Fruticosus | | | | | | | | | | | |
| (Blackberry) Leaf | | | | | | | | | | | |
| Extract | | | | | | | | | | | |
| SymMollient ® W/S | | | | | 2 | | 2 | | | | |
| Trideceth-9, PEG-5 | | | | | | | | | | | |
| Isononanoate, Water | | | | | | | | | | | |
| (Aqua) | | | | | | | | | | | |
| SymOcide ® C | | | | | | 0.1 | | | | | |
| o-Cymen-5-ol | | | | | | | | | | | |
| SymOcide ® PH | | | | | | | 1.0 | | | | |
| Phenoxyethanol, | | | | | | | | | | | |
| Hydroxyacetophenone, | | | | | | | | | | | |
| Caprylyl Glycol, | | | | | | | | | | | |
| Water (Aqua) | | | | | | | | | | | |
| SymOcide ® PS | | | | | | | | | | 0.8 | |
| Phenoxyethanol, | | | | | | | | | | | |
| Decylene Glycol, 1,2- | | | | | | | | | | | |
| Hexanediol | | | | | | | | | | | |
| SymOcide ® PT | | | | 0.8 | | | | | | | |
| Phenoxyethanol, | | | | | | | | | | | |
| Tropolone | | | | | | | | | | | |
| SymPeptide ® 225 | | | | 1 | | | | | | | |
| Glycerin, Water | | | | | | | | | | | |
| (Aqua), Myristoyl | | | | | | | | | | | |
| Pentapeptide-11 | | | | | | | | | | | |
| SymRelief ® 100 | | | | | | 0.2 | | | | | |
| Bisabolol, Zingiber | | | | | | | | | | | |
| Officinale (Ginger) | | | | | | | | | | | |
| Root Extract | | | | | | | | | | | |
| SymRelief S | | | | | | | | | | 0.1 | |
| Bisabolol, | | | | | | | | | | | |
| Hydroxymethoxyphenyl | | | | | | | | | | | |
| Decanone | | | | | | | | | | | |
| SymRepair ® 100 | | 1 | | 3 | | | | | | | |
| Hexyldecanol, | | | | | | | | | | | |
| Bisabolol, | | | | | | | | | | | |
| Cetylhydroxyproline | | | | | | | | | | | |
| Palmitamide, Stearic | | | | | | | | | | | |
| Acid, Brassica | | | | | | | | | | | |
| Campestris | | | | | | | | | | | |
| (Rapeseed) Sterols | | | | | | | | | | | |
| SymSave ® H | | 0.5 | | | 0.8 | 0.5 | | | | | 0.5 |
| Hydoxyacetophenone | | | | | | | | | | | |
| SymSol ® PF-3 | | | | | | 1.3 | | | | | |
| Water (Aqua), | | | | | | | | | | | |
| Pentylene Glycol, | | | | | | | | | | | |
| Sodium Lauryl | | | | | | | | | | | |
| Sulfoacetate, Sodium | | | | | | | | | | | |
| Oleoyl Sarcosinate, | | | | | | | | | | | |
| Sodium Chloride, | | | | | | | | | | | |
| Disodium | | | | | | | | | | | |
| Sulfoacetate, Sodium | | | | | | | | | | | |
| Oleate, Sodium | | | | | | | | | | | |
| Sulfate | | | | | | | | | | | |
| SymSitive ® 1609 | | | 0.5 | | | | | | 0.5 | | |
| Pentylene Glycol, 4-t- | | | | | | | | | | | |
| Butylcyclohexanol | | | | | | | | | | | |
| SymVital ® | | | 0.1 | | | | | | | | |
| AgeRepair 3040 | | | | | | | | | | | |
| Zingiber Officinale | | | | | | | | | | | |
| (Ginger) Root Extract | | | | | | | | | | | |
| SymWhite ® 377 | | | | | | | | | 0.5 | | |
| Phenylethyl | | | | | | | | | | | |
| Resorcinol | | | | | | | | | | | |
| Tamasterol ® | | | | | | | | | | 0.3 | |
| Phytosterols | | | | | | | | | | | |
| Tapioca Pure | | | | | | | | | | | 5 |
| Tapioca Starch | | | | | | | | | | | |
| Tegosoft ® PC 31 | | | | | | | | | | 0.3 | |
| Polyglyceryl-3 | | | | | | | | | | | |
| Caprate | | | | | | | | | | | |
| Triethanolamine | | | 0.3 | | | | | | | | |
| Vitamin A Palmitate | | | | 0.1 | | | | | | | |
| Retinyl Palmitate | | | | | | | | | | | |
| Vitamin E Acetate | | 0.5 | | 0.2 | | | | | | 0.3 | 0.5 |
| Tocopheryl Acetate | | | | | | | | | | | |
| Zetesol LA-2 | | | | | 26 | | | | | | |
| Ammonium Laureth | | | | | | | | | | | |
| Sulfate | | | | | | | | | | | |

| | |
|---|---|
| Water | ad 100 |

1 = Skin calming balm for sensitive skin
2 = Tinted anti-aging face balm, SPF 15
3 = After-sun moisturizing spray O/W
4 = Night cream W/O
5 = Skin cleansing gel
6 = After-shave hydrogel
7 = Anti-dandruff hair shampoo
8 = Anti-perspirant pump spray
9 = Skin lightening day care fluid O/W
10 = Skin barrier improving cream O/W
11 = Sun care lotion SPF 24 (UVA/UVB balance)

TABLE 14
Gel dental cream

| Ingredients | I (%) | II (%) | III (%) |
|---|---|---|---|
| Sodium carboxymethylcellulose | 0.40 | 0.40 | 0.40 |
| Sorbitol 70%, in water | 72.00 | 72.00 | 72.00 |
| Polyethylenglycol (PEG) 1500 | 3.00 | 3.00 | 3.00 |
| Sodium saccharinate | 0.07 | 0.07 | 0.07 |
| Sodium fluoride | 0.24 | 0.24 | 0.24 |
| p-Hydroxybenzoic acid (PHB) | 0.15 | 0.15 | |
| ethylester | | | |
| SymDiol 68 | | | 0.5 |
| SymSave H | | | 0.25 |
| Peppermint flavor | 1.00 | 1.00 | 1.00 |
| Avenanthramide L | | | 0.01 |
| Naked oat kernel extract in | 2.5 | | |
| glycerin/water standardized to | | | |
| 10-25 ppm Avn L | | | |
| Oat kernel extract on maltodextrin | | 0.5 | |
| standardized to ≥40 ppm Avn A | | | |
| and ≥10 ppm Avn L | | | |
| Abrasive Silica | 11.00 | 11.00 | 11.00 |
| Thickening Silica | 6.00 | 6.00 | 6.00 |
| Sodium dodecylsulfate (SDS) | 1.40 | 1.40 | 1.40 |
| Distilled water | ad 100.00 | ad 100.00 | ad 100.00 |

TABLE 15
Ready-to-use mouthwash with fluoride

| Ingredients | I (%) | II (%) | III (%) |
|---|---|---|---|
| Ethanol | 7.00 | 7.00 | |
| Glycerin | 12.00 | 12.00 | |
| Sodium fluoride | 0.05 | 0.05 | 0.18 |
| Pluronic F-127 ® | 1.40 | 1.40 | |
| (BASF, surface active substance) | | | |
| Sodium phosphate buffer pH 7.0 | 1.10 | 1.10 | |
| Sorbic acid | 0.20 | 0.20 | |
| Sodium saccharinate | 0.10 | 0.10 | 0.10 |
| Cinnamon/menthol flavor | 0.15 | 0.15 | 0.15 |
| Avenanthramide L | | | 0.005 |
| Naked oat kernel extract in | 2.00 | | |
| glycerine/water standardized to | | | |
| 10-25 ppm Avn L | | | |
| Oat kernel extract on maltodextrin | | 0.50 | |
| standardized to ≥40 ppm Avn A | | | |
| and ≥10 ppm Avn L | | | |
| Colour | 0.01 | 0.01 | 0.01 |
| Sorbitol 70% | | | 10 |
| Cremophor RH455 | | | 1.8 |
| SymDiol 68 | | | 0.5 |
| SymSave H | | | 0.2 |
| Distilled water | ad 100.00 | ad 100.00 | ad 100.00 |

TABLE 16
Chewing gum

| Ingredients | I (%) | II (%) | III (%) |
|---|---|---|---|
| Chewing gum base | 21.00 | 21.00 | 21.00 |
| Glucose syrup | 16.50 | 16.50 | 16.50 |
| Glycerin | 0.50 | 0.50 | 0.50 |
| Powdered sugar | 60.45 | 60.36 | 60.27 |
| Spearmint aroma | 1.50 | 1.50 | 1.50 |
| Avenanthramide L | 0.002 | | |
| Naked oat kernel extract on | | 0.5 | 0.2 |
| maltodextrin standardized | | | |
| to ≥30 ppm Avn L | | | |

TABLE 17
Sugar-free chewing gum against bad breath

| Ingredients | I (%) | II (%) | III (%) |
|---|---|---|---|
| Chewing gum base | 30.00 | 30.00 | 30.00 |
| Sorbitol, powder | 38.45 | 38.40 | 38.30 |
| Palatinite | 9.50 | 9.50 | 9.50 |
| Xylitol | 2.00 | 2.00 | 2.00 |
| Mannitol | 3.00 | 3.00 | 3.00 |
| Aspartame | 0.10 | 0.10 | 0.10 |
| Acesulfame K | 0.10 | 0.10 | 0.10 |
| Emulgum/emulsifier | 0.30 | 0.30 | 0.30 |
| Sorbitol 70%, in water | 14.00 | 14.00 | 14.00 |
| Glycerin | — | 0.50 | 0.75 |
| Cinnamon/menthol aroma | 1.50 | 1.50 | 1.50 |
| Avenanthramide L | | 0.002 | |
| Oat kernel extract in glycerin/ | 2.00 | | 0.50 |
| water standardized to 10-25 | | | |
| ppm Avn L | | | |

TABLE 18
Fruit gums

| Ingredients | I (%) | II (%) |
|---|---|---|
| Water | to 100 | to 100 |
| Saccharose | 34.50 | 34.50 |
| Glucose syrup, DE 40 | 31.89 | 31.89 |
| Iso Syrup C* Tru Sweet 01750 (Cerestar | 1.50 | 2.10 |
| GmbH) | | |
| Gelatine 240 Bloom | 8.20 | 9.40 |
| Colorant | 0.01 | 0.01 |
| Citric acid | 0.10 | 0.10 |
| Citrus flavor | 0.20 | — |
| Cherry flavor | — | 0.1 |
| Avenanthramide L | | 0.003 |
| Naked oat kernel extract on maltodextrin | 0.5 | |
| standardized to ≥50 ppm Avn L | | |

TABLE 19
Yoghurt with low fat content

| Ingredients | I (%) | II (%) | III (%) |
|---|---|---|---|
| Sucrose | 110 | 8 | — |
| Sucralose | — | 0.02 | 0.2 |
| Saccharin | — | | 0.3 |
| Sour cherry extract | 0.2 | 0.1 | 0.2 |
| Cherry flavor | — | 0.01 | — |
| Avenanthramide L | — | 0.01 | — |
| Oat kernel extract on maltodextrin | 0.05 | — | 2.0 |
| standardized to ≥100 ppm Avn L | | | |
| Yoghurt, 0.1% fat | ad 100 | ad 100 | ad 100 |

### Example 10: Synthesis of Avenanthramide L

### Step 1: Synthesis of methyl (2E)-4-(diethoxyphosphoryl)but-2-enoate

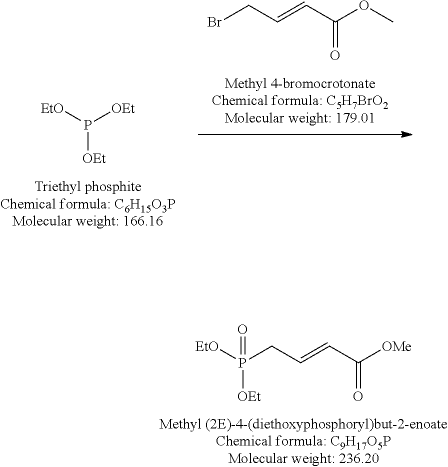

Experimental Procedure:

Methyl 4-bromocrotonate (15.57 ml, 132.4 mmol, 1.0 eq) and triethyl phosphite (22.70 ml, 132.4 mmol, 1.0 eq) were added to a round-bottomed flask and heated at reflux with stirring for 4 hours. The RM was then cooled down to room temperature. TLC analysis (Hex:EtOAc, 1:1) confirmed the consumption of starting materials and formation of the desired product.

Work Up:

No additional work up was performed.

Purification:

The reaction mixture was poured onto silica and purified by column chromatography eluted with Hex:EtOAc (20 to 100%). The pure product was concentrated to dryness, yielding 21.7 g (68%) of methyl (2E)-4-(diethoxyphosphoryl)but-2-enoate (3).

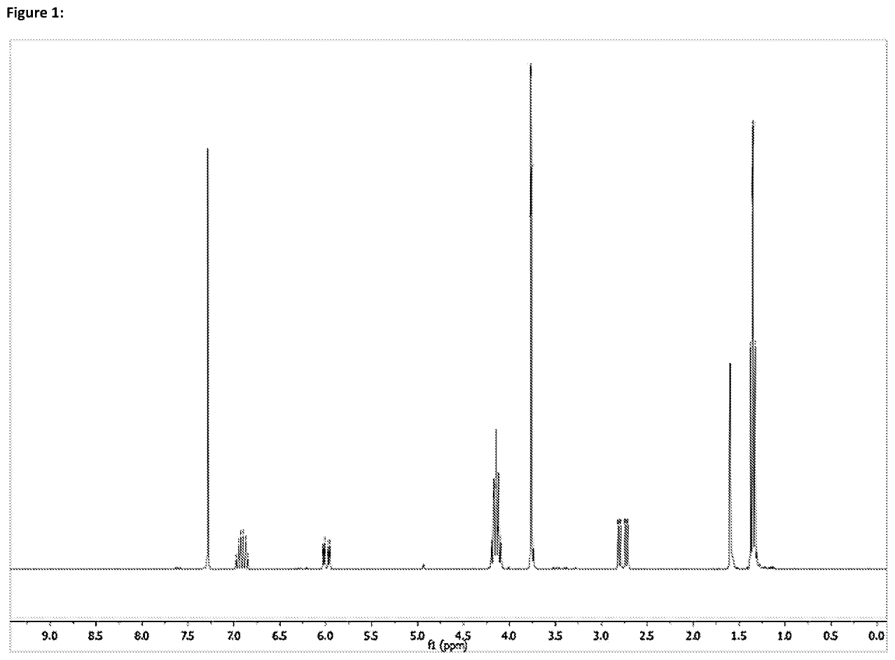

FIG. 1 shows the ¹H NMR spectrum of (3), CDCl₃, 300 MHz. 6.98-6.85 (m, 1H), 5.99 (d, J=15 Hz, 1H), 4.20-4.10 (m, 8 Hz, 4H), 3.77 (s, 3H), 2.77 (dd, J=24 Hz, 8 Hz, 2H), 1.35 (t, J=8 Hz, 3H).

### Step 2: Synthesis of methyl (2E,4E)-5-(4-hydroxyphenyl)penta-2,4-dienoate

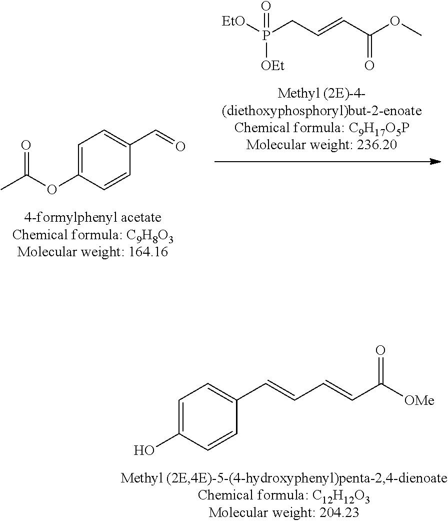

Experimental Procedure:

A solution of methyl (2E)-4-(diethoxyphosphoryl)but-2-enoate (5.7 g, 24.17 mmol, 1.0 eq) in dry THF was added dropwise to a three-neck round-bottomed flask containing NaH (4.021 g, 100.57 mmol, 4.16 eq) in dry THF placed in a low-temperature reactor under an argon atmosphere. The mixture was stirred at −50° C. for 0.5 hours, following which a solution of 4-formylphenyl acetate (3.294 g, 20.06 mmol, 0.83 eq) in dry THF was added dropwise. The reaction temperature was raised to 0° C., and the mixture was stirred for another 2 hours.

Work Up:

The reaction mixture was quenched with an NH₄Cl saturated solution, followed by extraction with EtOAc. Organic layers were combined, dried over Na₂SO₄ and concentrated to dryness.

Purification:

The crude product (1 vol.) was triturated with MeOH (10 vol.). A precipitate was formed and filtered on a filter funnel, yielding avenalumic acid methyl ester (3.17 g, 77%) (5).

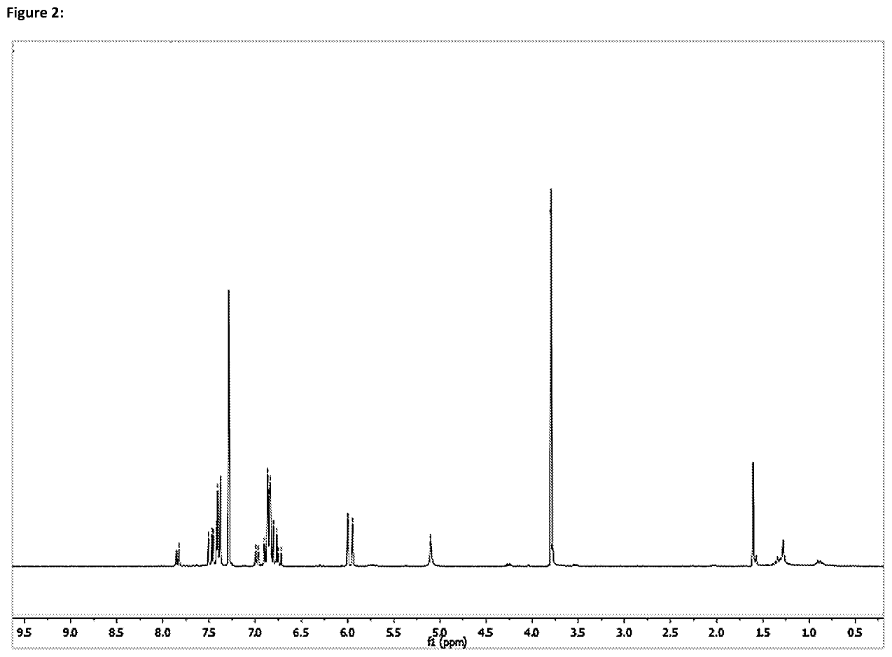

FIG. 2 shows the ¹H NMR spectrum of (5), CDCl₃, 300 MHz. 7.82-7.41 (m, 4H), 6.99-6.72 (m, 3H), 5.97 (d, J=15 Hz, 1H), 5.10 (br s, 1H), 3.80 (s, 3H).

### Step 3: Synthesis of (2E,4E)-5-(4-hydroxyphenyl)penta-2,4-dienoic acid

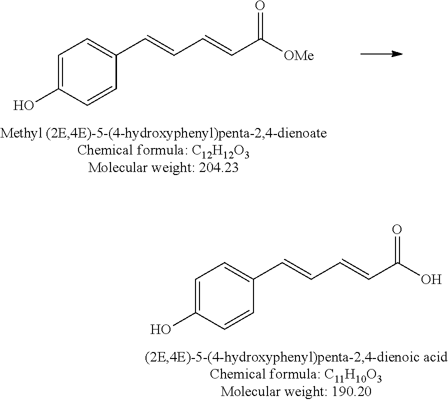 [SMILES: COC(=O)/C=C/C=C/c1ccc(O)cc1 ; O=C(O)/C=C/C=C/c1ccc(O)cc1]

Experimental Procedure:

Methyl (2E,4E)-5-(4-hydroxyphenyl)penta-2,4-dienoate (4.14 g, 20.27 mmol, 1.0 eq) was dissolved in MeOH (165.6 g, 40.0 vol.), then 1M NaOH (165.6 g, 40.0 vol.) was added. The reaction was stirred overnight at room temperature.

Work Up:

MeOH was evaporated. The crude product was acidified with 1M HCl, and extraction was performed using EtOAc. Organic layers were combined, dried over Na₂SO₄ and concentrated to dryness, yielding avenalumic acid (3.8 g, 99% Avn Ac).

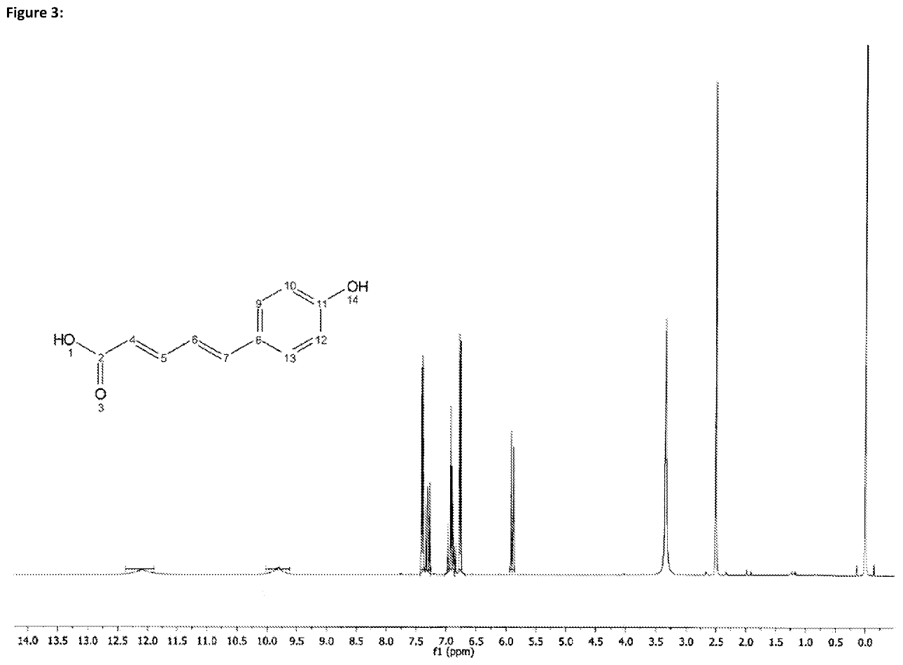

FIG. 3 shows the ¹H NMR spectrum of avenalumic acid (Avn Ac), DMSO-d₆, 400 MHz. 12.10 (s, 1H), 9.81 (s, 1H), 7.43-7.36 (m, 2H), 7.31 (dd, J=15.2, 10.2 Hz, 1H), 6.95 (d, J=15.6 Hz, 1H), 6.88 (dd, J=15.5, 10.3 Hz, 1H), 6.80-6.74 (m, 2H), 5.90 (d, J=15.1 Hz, 1H), 1.23 (s, 1H).

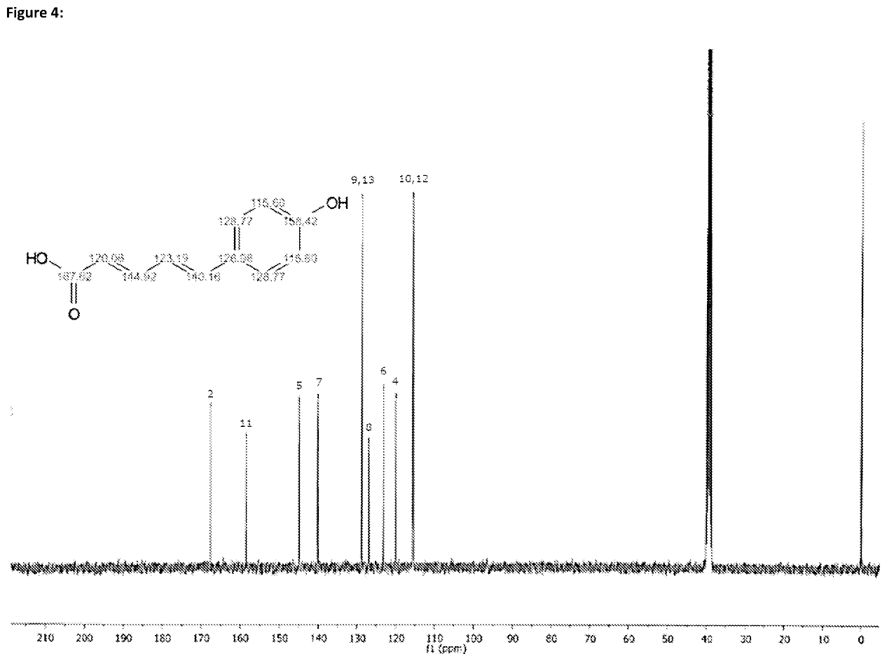

FIG. 4 shows the ¹³C NMR spectrum of avenalumic acid (Avn Ac), DMSO-d₆, 101 MHz. 167.62, 158.42, 144.92, 140.16, 128.77, 126.98, 123.19, 120.06, 115.60, 40.09, 40.03, 39.82, 39.62, 39.41, 39.20, 38.99, 38.78.

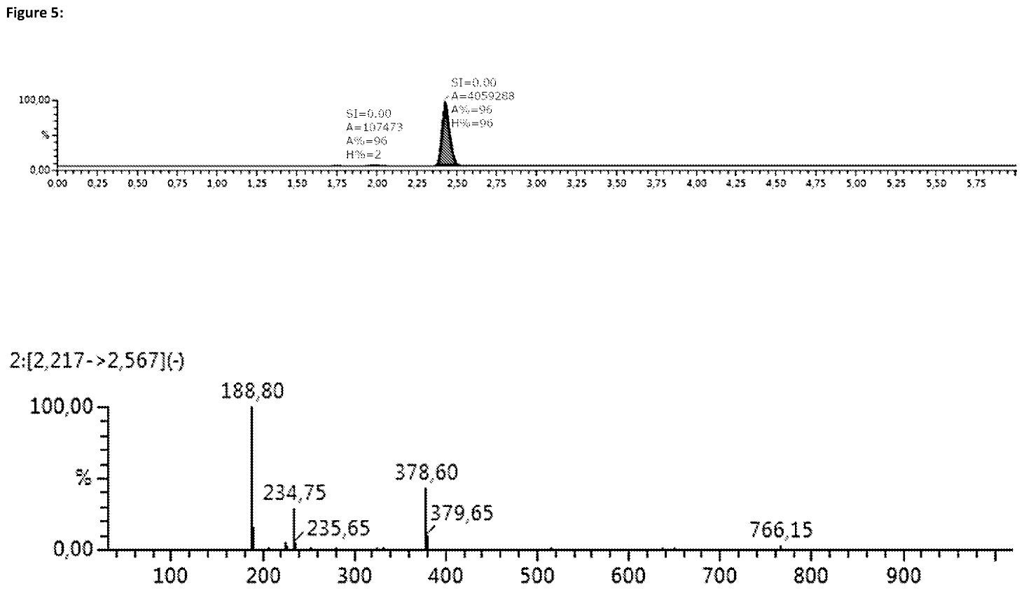

FIG. 5 shows the LCMS spectrum of avenalumic acid for m/z−1=188.8 using a Gemini-NX 3 μM C18 (4.6×50 mm), column gradient flow 0.5 ml/min, 0 min→2 min, using 95% water/5% MeCN; 2 min→9.5 min, linear gradient from 95 to 20% water and from 5% to 80% MeCN, then hold this for 1 min., modifier formic acid 0.1% of each solvent.

### Step 4: Synthesis of 5-hydroxy-2-[(2E,4E)-5-(4-hydroxyphenyl)penta-2,4-dienamido]benzoic acid

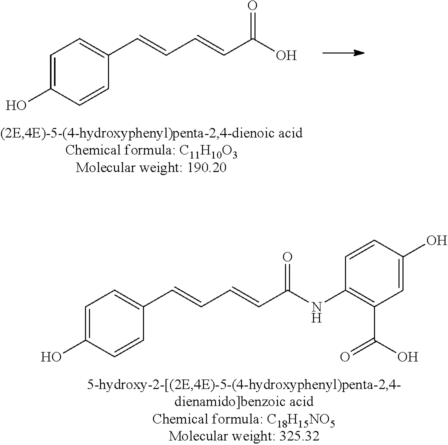 [SMILES: O=C(O)/C=C/C=C/c1ccc(O)cc1 ; O=C(/C=C/C=C/c1ccc(O)cc1)Nc1ccc(O)cc1C(=O)O]

Experimental Procedure 1:

(2E,4E)-5-(4-hydroxyphenyl)penta-2,4-dienoic acid (0.1 g, 0.53 mmol, 1.0 eq), COMU (0.27 g, 0.63 mmol, 1.1 eq) and DIPEA (0.41 g, 3.17 mmol, 6.0 eq) were dissolved in DMF (5 ml). The reaction mixture was stirred for 10 minutes, then 2-amino-5-hydroxybenzoic acid (0.08 g, 0.53 mmol, 1.0 eq) was added. The reaction mixture was stirred overnight at room temperature. LCMS analysis confirmed the consumption of the starting material and formation of a new product.

Or Alternatively:

Experimental Procedure 2 (Preferred):

(2E,4E)-5-(4-hydroxyphenyl)penta-2,4-dienoic acid (1.55 g, 8.15 mmol, 1.0 eq), HOBt (1.21 g, 8.96 mmol, 1.1 eq), EDC HCl (1.71 g, 8.96 mmol, 1.1 eq) and DIPEA (7.11 ml, 40.75 mmol, 5.0 eq) were dissolved in DMF (38.75 ml). The reaction mixture was stirred for 10 minutes, then 2-amino-5-hydroxybenzoic acid (1.24 g, 8.15 mmol, 1.0 eq) was added. The reaction mixture was stirred overnight at room temperature. LCMS analysis confirmed the consumption of the starting material and formation of a new product.

Work-Up:

EtOAc was added to the reaction mixture, and the mixture was washed with 1M HCL (5×100 ml). The organic layer was dried over Na₂SO₄ and concentrated to dryness.

Purification:

The crude product was purified via recrystallisation (water/methanol) or on preparative HPLC using a Gemini-NX 5 μM C18 (250×21.2 mm), column gradient flow 20 ml/min, using water and acetonitrile with 0.1% formic acid as modifier. 67% water, 0 min→15 min, 50% water; 15 min→16 min, 5% water; hold for 4 minutes, yielding 250 mg (12%) of avenanthramide L.

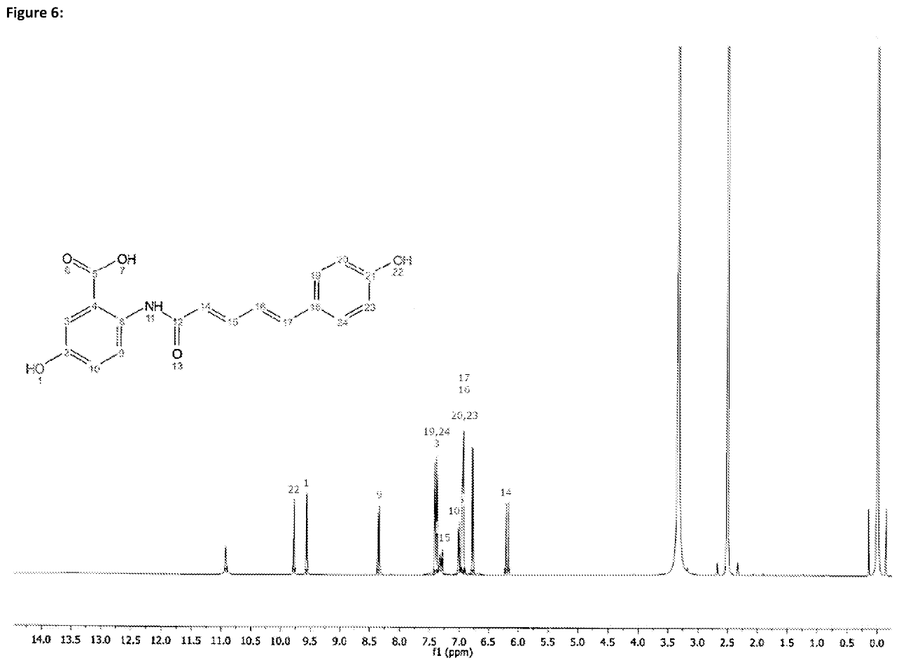

FIG. 6 shows the ¹H NMR spectrum of avenanthramide L, DMSO-d₆, 400 MHz. 10.92 (s, OH), 9.78 (s, 1H), 9.57 (s, 1H), 8.35 (d, J=9.0 Hz, 1H), 7.44-7.38 (m, 2H), 7.37 (d, J=2.9 Hz, 1H), 7.31 (ddd, J=14.9, 7.3, 3.0 Hz, 1H), 7.01 (dd, J=9.0, 3.0 Hz, 1H), 6.94 (d, J=7.3 Hz, 1H), 6.94 (d, J=3.2 Hz, 1H), 6.80-6.75 (m, 2H), 6.20 (d, J=14.8 Hz, 1H).

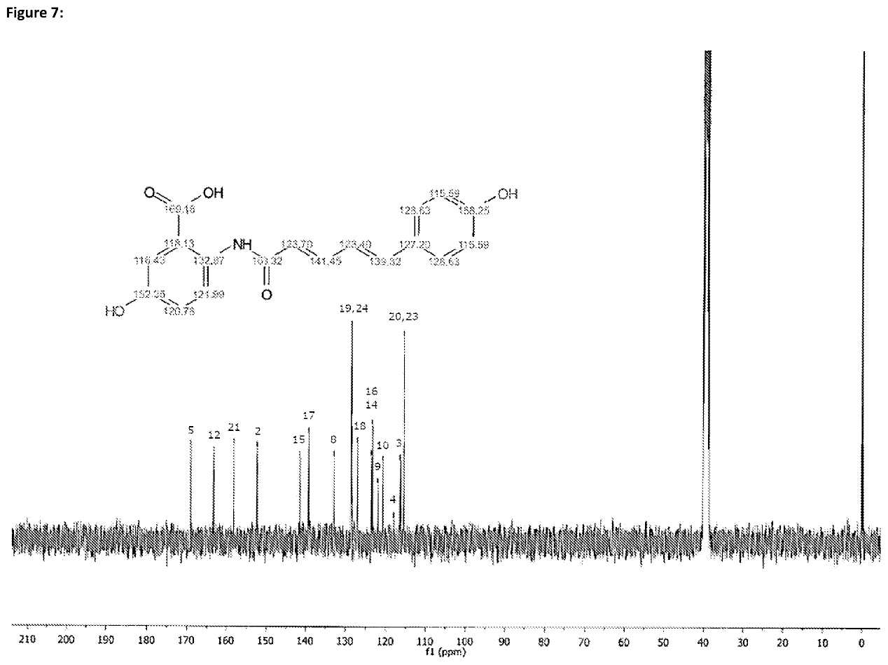

FIG. 7 shows the ¹³C NMR spectrum of avenanthramide L, DMSO-d₆, 101 MHz. 169.18, 163.32, 158.25, 152.35, 141.45, 139.32, 132.87, 128.63, 127.20, 123.70, 123.40, 121.99, 120.78, 118.13, 116.43, 115.59, 40.09, 40.04, 39.83, 39.62, 39.42, 39.21, 39.00, 38.79, 0.00.

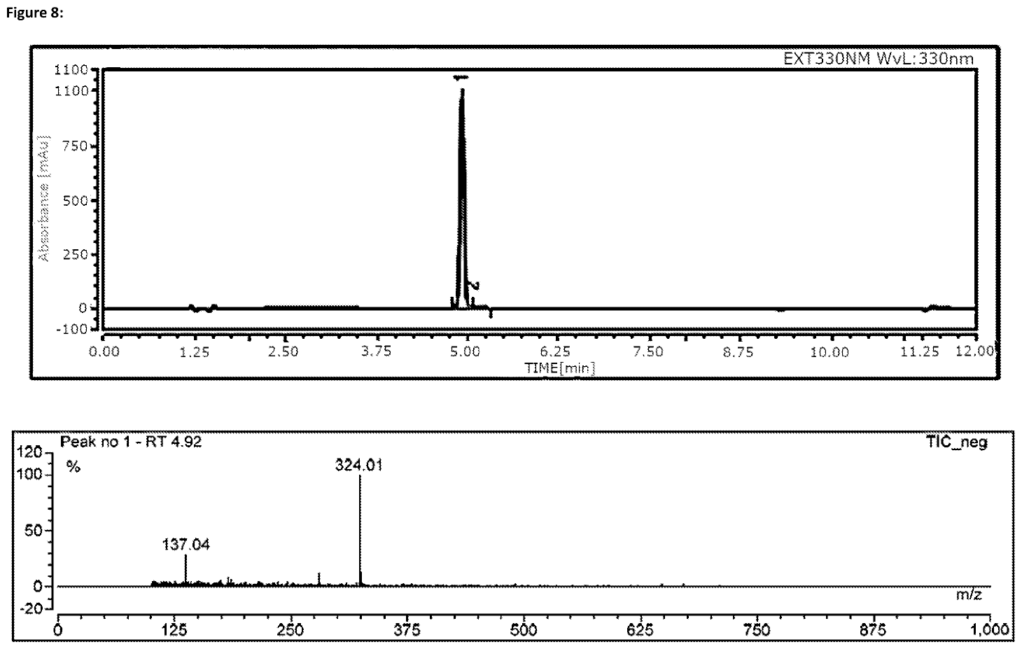

FIG. 8 shows the LCMS spectrum of avenanthramide L (m/z−1=324.01).

1. The method of claim 6, comprising using avenanthramide L or an oat extract comprising avenanthramide L as an antagonist of the neurokinin-1 receptor NK1R, wherein the administering comprises applying the avenanthramide L or an oat extract comprising avenanthramide L to skin, hair, or nails of a subject in need thereof.

2. A method of claim 6, comprising using avenanthramide L or an oat extract comprising avenanthramide L for inducing the expression of small heat shock proteins (sHSPs) or for inducing the expression of CD44, wherein the administering comprises applying the avenanthramide L or an oat extract comprising avenanthramide L to skin, hair, or nails of a subject in need thereof.

3. The method according to claim 2, wherein the small heat shock proteins (sHSPs) are selected from the group of sHSP27 (HSPB1, HSPB2, HSPB3) and αB-crystallin (CRYAB/HSPB5).

4. of the method of claim 6 comprising using avenanthramide L or an oat extract comprising avenanthramide L as an antioxidant agent or for inducing the expression of BLVRB, wherein the administering comprises applying the avenanthramide L or an oat extract comprising avenanthramide L to skin, hair, or nails of a subject in need thereof.

5. A method of using avenanthramide L or oat extract comprising avenanthramide L as a cosmetic for skin care, hair care or nail care and/or for use in the prevention and/or treatment of sensitive skin, hair or nails, skin irritation, skin reddening, wheals, pruritis (itching), skin aging, wrinkle formation, loss of skin volume, loss of skin elasticity, pigment spots, pigment abnormalities, dry skin, or for moisturising the skin, comprising administering the avenanthramide L or oat extract comprising avenanthramide L to a subject in need thereof.

6. A method of treatment using avenanthramide L or an oat extract comprising avenanthramide L as a medicament comprising administering the avenanthramide L or an oat extract comprising avenanthramide L to a subject in need thereof.

7. The method according to claim 6, comprising using the avenanthramide L or an oat extract comprising avenanthramide L in the prevention and/or treatment of dermatological or keratological diseases wherein the administering comprises applying the avenanthramide L or an oat extract comprising avenanthramide L to skin, hair, or nails of a subject in need thereof.

8. The method according to claim 7, wherein the dermatological diseases are selected from the group of eczema, psoriasis, seborrhoea, dermatitis, erythema, pruritis (itching), otitis, xerosis, inflammation, irritation, fibrosis, Lichen planus, Pityriasis rosea, Pityriasis versicolor, autoimmune bullous diseases, urticarial, angiodermal and allergic skin reactions, and wound healing.

9. A food, food supplement, cosmetic, pharmaceutical, or veterinary preparation comprising avenanthramide L or an oat extract comprising avenanthramide L.

10. The method according to claim 5, wherein the oat extract is an extract from plants of the genus Avena and/or wherein the extract is an aqueous-alcoholic or aqueous-acetonic extract.

11. The method according to claim 5, wherein the avenanthramide L or the oat extract comprising avenanthramide L is used in combination with at least one naturally occurring analogue avenanthramide other than avenanthramide L, and/or wherein the avenanthramide L or the oat extract comprising avenanthramide L is used in combination with at least one non-naturally occurring analogue avenanthramide.

12. The method according to claim 5, wherein the avenanthramide L or the oat extract comprising avenanthramide L is used in combination with:
an anti-inflammatory, antibacterial or antimycotic substance; and/or
a substance having a reddening-alleviating or itch-alleviating action; and/or
a lenitive substance; and/or
a moisturiser regulator; and/or
a cooling agent.

13. The method according to claim 5, wherein the avenanthramide L or the oat extract comprising avenanthramide L is used in combination with an excipient selected from the group consisting of antioxidants, preservatives, (metal) chelating agents, penetration enhancers and/or a mixture of any of the foregoing.

14. The method according to claim 5, wherein the administering is topical.

15. The method according to claim 9, wherein the food, food supplement, cosmetic, pharmaceutical, or veterinary preparation comprises the avenanthramide L or the oat extract comprising avenanthramide L in an amount of 0.0001 to 10 wt %, based on the total weight of the preparation.

16. The method of claim 6, comprising using the avenanthramide L or oat extract comprising avenanthramide L as an antagonist of the neurokinin-1 receptor NK1R, wherein the administering comprises applying the avenanthramide L or an oat extract comprising avenanthramide L to skin, hair, or nails of a subject in need thereof.

17. A method for preparing avenalumic acid and/or avenanthramide L, comprising the steps of:
(a) reacting triethyl phosphite (1) and methyl 4-bromocrotonate (2) to form methyl (2E)-4-(diethylphosphoryl)but-2-enoate (3);
(b) reacting methyl (2E)-4-(diethylphosphoryl)but-2-enoate (3) in an HWE reaction with 4-formylphenyl acetate (4) to form methyl (2E, 4E)-5-(4-hydroxyphenyl)penta-2,4-dienoate (5);
(c) deprotecting avenalumic acid methyl ester (5), using a sodium hydroxide solution, to yield avenalumic acid (Avn Ac); and
(d) reacting avenalumic acid (Avn Ac) with 2-amino-5-hydroxybenzoic acid (6), using coupling reagents and without using any protecting groups, to yield avenanthramide L (Avn L).

18. The method according to claim 17, wherein step (b) is performed at a temperature in a range of −78° C. to 0° C.

19. The method according to claim 18, wherein step (b) is performed at a temperature in a range of −50° C. to 0° C.

20. The method according to claim 10, wherein the oat extract is an extract from plants of the oat species Avena sativa or Avena nuda.
